(19) United States
(12) Patent Application Publication
(10) Pub. No.: US 2024/0335556 A1
(43) Pub. Date: Oct. 10, 2024

(54) Compounds and Methods for Reducing DMPK Expression

(71) Applicant: Ionis Pharmaceuticals, Inc., Carlsbad, CA (US)

(72) Inventors: Frank Rigo, Carlsbad, CA (US); Chrissa A. Dwyer, Collegeville, PA (US)

(73) Assignee: Ionis Pharmaceuticals, Inc., Carlsbad, CA (US)

(21) Appl. No.: 18/486,504

(22) Filed: Oct. 13, 2023

Related U.S. Application Data
(63) Continuation of application No. 17/823,854, filed on Aug. 31, 2022, now Pat. No. 11,833,221.
(60) Provisional application No. 63/239,765, filed on Sep. 1, 2021.

(51) Int. Cl. A61K 47/68; A61K 47/54; C12N 15/113

(52) U.S. Cl. CPC A61K 47/6849; A61K 47/549; A61K 47/6807; C12N 15/1137; C12N 2310/315; C12N 2310/3231

(57) ABSTRACT

Provided are oligomeric compounds, methods, and pharmaceutical compositions for DMPK the amount or activity of DMPK RNA in a cell or animal, and in certain instances reducing the amount of DMPK protein in a cell or animal. Such oligomeric compounds, methods, and pharmaceutical compositions are useful to treat type 1 myotonic dystrophy.

### SEQUENCE LISTING

The present application is being filed along with a Sequence Listing in electronic format. The Sequence Listing is provided as an XML file entitled BIOL0441USCISEQ.xml, created on Dec. 7, 2023, which is 247,970 bytes in size. The information in the electronic format of the sequence listing is incorporated herein by reference in its entirety.

### FIELD

Provided are oligomeric compounds, methods, and pharmaceutical compositions for reducing the amount or activity of DMPK RNA in a cell or animal, and in certain instances reducing the amount of DMPK protein in a cell or animal. Such oligomeric compounds, methods, and pharmaceutical compositions are useful to treat type 1 myotonic dystrophy (DM1) in an animal.

### BACKGROUND

Myotonic dystrophy type 1 (DM1) is the most common form of muscular dystrophy in adults with an estimated frequency of 1 in 7,500 (Harper P S., Myotonic Dystrophy. London: W. B. Saunders Company; 2001). DM1 is an autosomal dominant disorder caused by expansion of a non-coding CTG repeat in DMPK1. DMPK1 is a gene encoding a cytosolic serine/threonine kinase (Brook J D, et al., Cell., 1992, 68(4):799-808). The physiologic functions and substrates of this kinase have not been fully determined. The expanded CTG repeat is located in the 3′ untranslated region (UTR) of DMPK1. This mutation leads to RNA dominance, a process in which expression of RNA containing an expanded CUG repeat (CUGexp) induces cell dysfunction (Osborne R J and Thornton C A., Human Molecular Genetics., 2006, 15(2): R162-R169).

The DMPK gene normally has 5-37 CTG repeats in the 3′ untranslated region. In myotonic dystrophy type 1, this number is significantly expanded and is, for example, in the range of 50 to greater than 3,500 (Harper, Myotonic Dystrophy (Saunders, London, ed.3, 2001); Annu. Rev. Neurosci. 29: 259, 2006; EMBO J. 19: 4439, 2000; Curr Opin Neurol. 20; 572, 2007).

The CUGexp tract interacts with RNA binding proteins including muscleblind-like (MBNL) protein, a splicing factor, and causes the mutant transcript to be retained in nuclear foci. The toxicity of this RNA stems from sequestration of RNA binding proteins and activation of signaling pathways. Studies in animal models have shown that phenotypes of DM1 can be reversed if toxicity of CUGexp RNA is reduced (Wheeler T M, et al., Science., 2009, 325(5938):336-339; Mulders S A, et al., Proc Natl Acad Sci U S A., 2009, 106(33): 13915-13920).

In DM1, skeletal muscle is the most severely affected tissue, but the disease also has important effects on cardiac and smooth muscle, ocular lens, and brain. The cranial, distal limb, and diaphragm muscles are preferentially affected. Manual dexterity is compromised early, which causes several decades of severe disability. The median age at death is 55 years, usually from respiratory failure (de Die-Smulders CE, et al., Brain, 1998, 121(Pt 8): 1557-1563).

Antisense technology is emerging as an effective means for modulating expression of certain gene products and may therefore prove to be uniquely useful in a number of therapeutic, diagnostic, and research applications for the modulation of DMPK1.

Presently there is no treatment that can modify the course of DM1. The burden of disease, therefore, is significant. It is, therefore, an object herein to provide compounds, compositions, and methods for treating DM1.

### SUMMARY

Oligomeric compounds, methods, and pharmaceutical compositions of certain embodiments described herein are useful for reducing or inhibiting DMPK expression in a cell or animal. In certain embodiments, DMPK RNA or protein levels can be reduced in a cell or animal. In certain embodiments, the subject has type 1 myotonic dystrophy (DM1). In certain embodiments, the subject has a disease or disorder associated with a mutation in DMPK.

Also provided are methods of treating an animal having type 1 myotonic dystrophy.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH2 CH2OCH3 group in place of the 2′-OH group of a furanosyl sugar moiety. A “2′-MOE sugar moiety” means a sugar moiety with a 2′-OCH₂CH₂OCH₃ group in place of the 2′-OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” means a nucleoside comprising a 2′-MOE sugar moiety.

As used herein, “2′-OMe” means a 2′-OCH₃ group in place of the 2′-OH group of a furanosyl sugar moiety. A “2′-O-methyl sugar moiety” or “2′-OMe sugar moiety” means a sugar moiety with a 2′-OCH₃ group in place of the 2′-OH group of a furanosyl sugar moiety. Unless otherwise indicated, a 2′-OMe sugar moiety is in the β-D-ribosyl configuration.

As used herein, “2′-OMe nucleoside” means a nucleoside comprising a 2′-OMe sugar moiety.

As used herein, “5-methylcytosine” means a cytosine modified with a methyl group attached to the 5 position. A 5-methylcytosine is a modified nucleobase.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is one or more of muscle stiffness, myotonia, disabling distal weakness, weakness in face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts.

As used herein, “antisense agent” means an antisense compound and optionally one or more additional features, such as a sense compound.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) of cerebrospinal fluid and is biocompatible with CSF.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “constrained ethyl” or “cEt” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein, “deoxy region” means a region of 5-12 contiguous nucleotides, wherein at least 70% of the nucleosides comprise a β-D-2′-deoxyribosyl sugar moiety. In certain embodiments, a deoxy region is the gap of a gapmer.

As used herein, “internucleoside linkage” is the covalent linkage between adjacent nucleosides in an oligonucleotide. As used herein “modified internucleoside linkage” means any internucleoside linkage other than a phosphodiester internucleoside linkage.

As used herein, “linked nucleosides” are nucleosides that are connected in a contiguous sequence (i.e., no additional nucleosides are presented between those that are linked).

As used herein, “motif” means the pattern of unmodified and/or modified sugar moieties, nucleobases, and/or internucleoside linkages, in an oligonucleotide.

As used herein, “modified nucleoside” means a nucleoside comprising a modified nucleobase and/or a modified sugar moiety.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution, or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “prodrug” means an inactive or less active form of a compound which, when administered to a subject, is metabolized to form the active, or more active, compound. In certain embodiments, a prodrug comprises a cell-targeting moiety and at least one active compound.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is random when it is the result of a synthetic method that is not designed to control the stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center (“racemic”). In certain embodiments, the stereorandom chiral center is not racemic because one absolute configuration predominates following synthesis, e.g., due to the action of non-chiral reagents near the enriched stereochemistry of an adjacent sugar moiety. In certain embodiments, the stereorandom chiral center is at the phosphorous atom of a stereorandom phosphorothioate internucleoside linkage.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an oligomeric compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNAse H cleavage. The internal region may be referred to as the “gap” and the external regions may be referred to as the “wings.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. As used herein, the term “MOE gapmer” indicates a gapmer having a gap comprising 2′-β-D-deoxynucleosides and wings comprising 2′-MOE nucleosides. Unless otherwise indicated, a gapmer may comprise one or more modified internucleoside linkages and/or modified nucleobases and such modifications do not necessarily follow the gapmer pattern of the sugar modifications.

As used herein, “hybridization” means the annealing of oligonucleotides and/or nucleic acids. While not limited to a particular mechanism, the most common mechanism of hybridization involves hydrogen bonding, which may be Watson-Crick. Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments. RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “treating” means improving a subject's disease or condition by administering an oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to an animal. For example, a therapeutically effective amount improves a symptom of a disease.

### CERTAIN EMBODIMENTS

Embodiment 1. An oligomeric compound comprising a modified oligonucleotide according to the following chemical notation: TₖₛTₖₒ^{m}Cₖₒ^{m}C_{ds}C_{ys}G_{ds}A_{ds}A_{ds}T_{ds}G_{ds}T_{ds}^{m}C_{ds}^{m}C_{ds}GₖₒAₖₛ^{m}Cₖ (SEQ ID NO: 13), wherein:
A=an adenine nucleobase,
^{m}C=a 5-methylcytosine nucleobase,
C=a cytosine nucleobase,
G=a guanine nucleobase,
T=a thymine nucleobase,
y=a 2′-OMe sugar moiety,
k=a cEt sugar moiety,
d =a 2′-β-D-deoxyribosyl sugar moiety,
s=a phosphorothioate internucleoside linkage, and
o=a phosphodiester internucleoside linkage.

Embodiment 2. The oligomeric compound of embodiment 1 comprising a conjugate group.

Embodiment 3. The oligomeric compound of embodiment 2, wherein the conjugate group comprises a conjugate moiety and a conjugate linker.

Embodiment 4. The oligomeric compound of embodiment 2, wherein the conjugate group comprises C₁₀-C₂₄ alkyl.

Embodiment 5. The oligomeric compound of embodiment 2, wherein the conjugate group comprises C₁₆ alkyl.

Embodiment 6. The oligomeric compound of embodiment 3, wherein the conjugate moiety is a cell-targeting moiety.

Embodiment 7. The oligomeric compound of embodiment 3, wherein the cell-targeting moiety is selected from a carbohydrate, an antibody, and an antibody fragment.

Embodiment 8. The oligomeric compound of any of embodiments 6-7, wherein the cell-targeting moiety binds a cell surface receptor on a skeletal muscle cell.

Embodiment 9. The oligomeric compound of any of embodiments 6-8, wherein the cell-targeting moiety is an antibody or an antibody fragment that binds a transferrin receptor.

Embodiment 10. A modified oligonucleotide according to the following chemical structure:

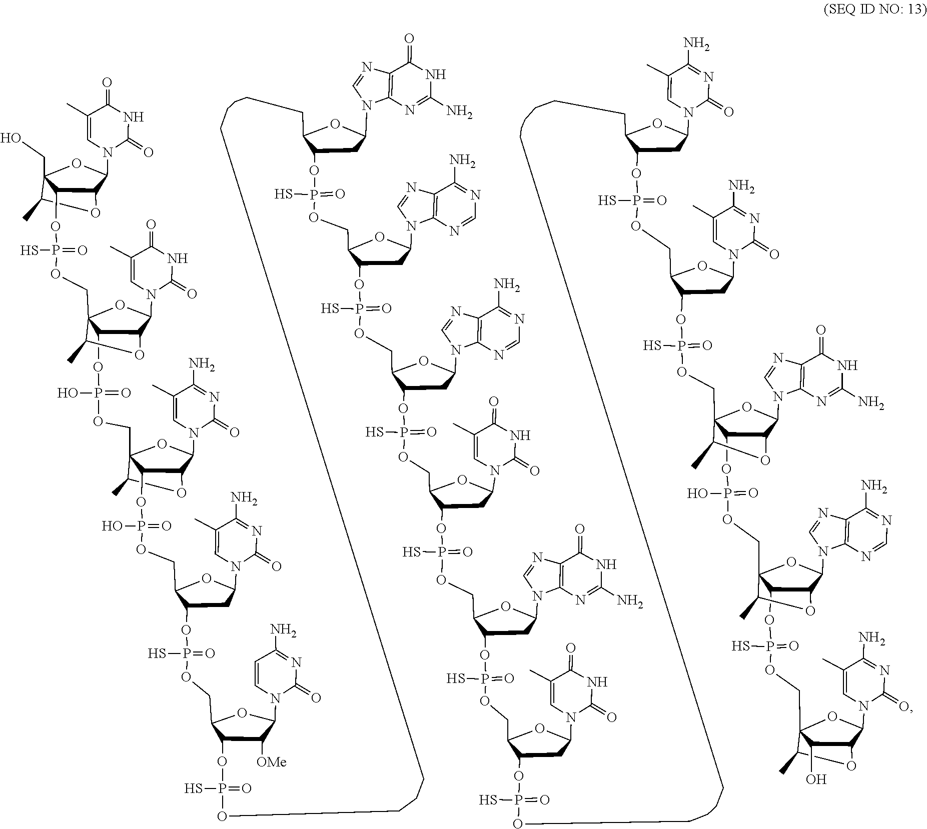

or a salt thereof.

Embodiment 11. The modified oligo nucleotide of embodiment 10. which is a sodium salt or a potassium salt.

Embodiment 12. A modified oligonucleotide according to the following chemical structure:

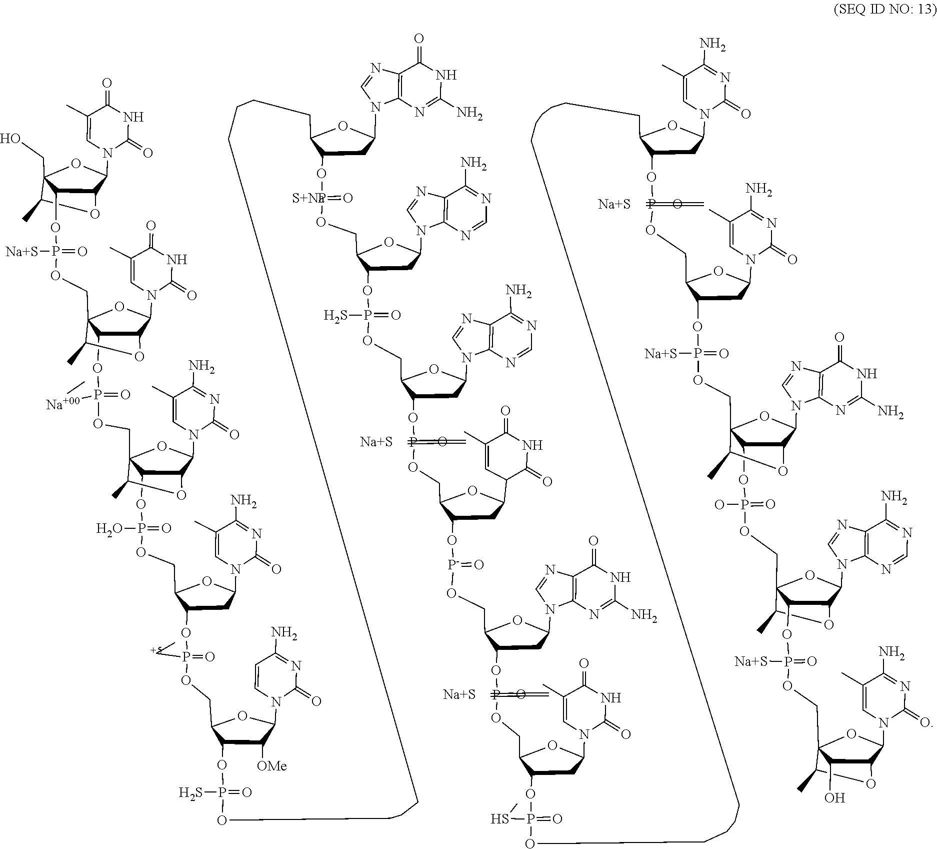

Embodiment 13. An oligomeric compound according to the following chemical structure:

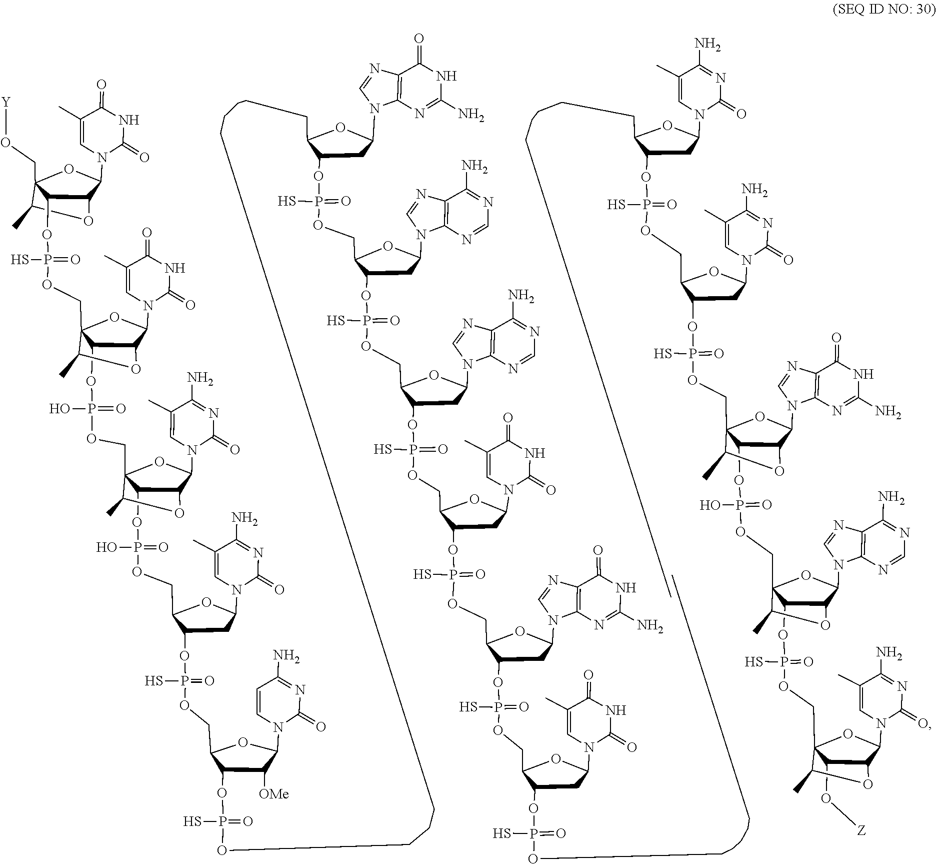

or a salt thereof, wherein Y and Z are selected from H and a conjugate group, wherein at least one of Y and Z is a conjugate group.

Embodiment 14. The oligomeric compound of embodiment 13, which is a sodium salt or a potassium salt.

Embodiment 15. An oligomeric compound according to the following chemical structure:

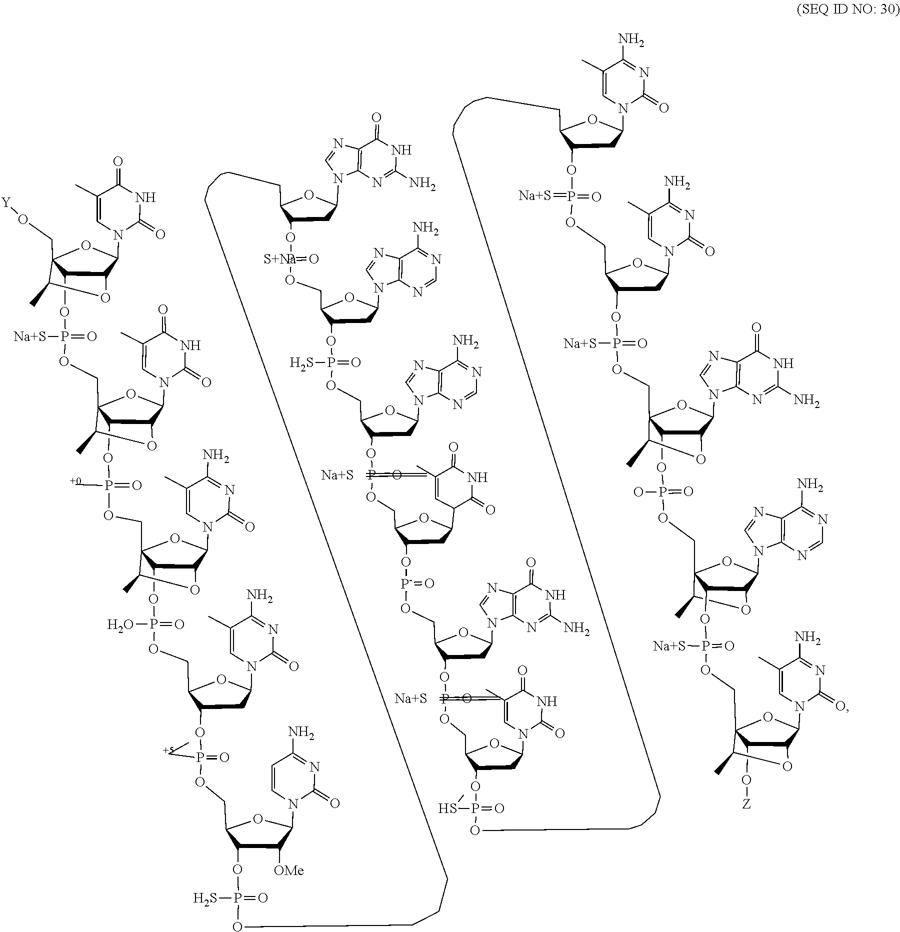

wherein Y and Z are selected from H and a conjugate group, wherein at least one of Y and Z is a conjugate group.

Embodiment 16. The oligomeric compound of any of embodiments 13-15, wherein the conjugate group comprises a conjugate moiety and a conjugate linker.

Embodiment 17. The oligomeric compound of any of embodiments 13-15, wherein the conjugate group comprises C₁₀-C₂₄ alkyl.

Embodiment 18. The oligomeric compound of any of embodiments 13-15, wherein the conjugate group comprises C₁₆.

Embodiment 19. The oligomeric compound of embodiment 16, wherein the conjugate moiety is a cell-targeting moiety.

Embodiment 20. The oligomeric compound of embodiment 19, wherein the cell-targeting moiety binds a cell surface receptor on a skeletal muscle cell.

Embodiment 21. The oligomeric compound of embodiment 19 or embodiment 20, wherein the cell-targeting moiety is selected from a carbohydrate and an antibody.

Embodiment 22. The oligomeric compound of embodiment 19 or embodiment 20, wherein the cell-targeting moiety is an antibody or an antibody fragment that binds a transferrin receptor.

Embodiment 23. The oligomeric compound of embodiment 19, wherein the cell-targeting moiety comprises a GalNAc.

Embodiment 24. The oligomeric compound of any of embodiments 13-15, wherein Y is:

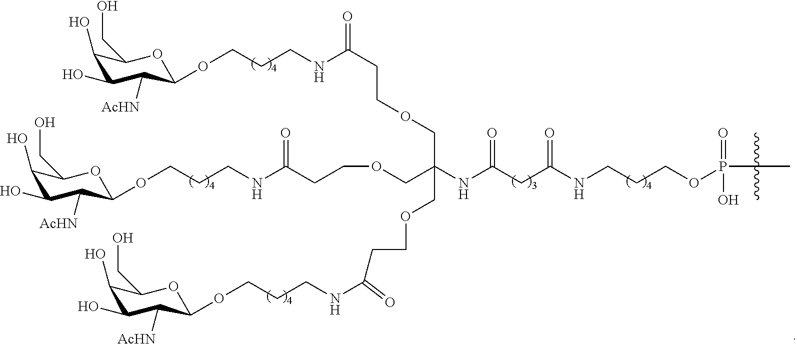

Embodiment 25. A prodrug of the oligomeric compound of any of embodiments 1-9 and 13-24 or the modified oligonucleotide of any of embodiments 10-12.

Embodiment 26. The oligomeric compound of any of embodiments 1-9 and 13-24, wherein the oligomeric compound is a prodrug.

Embodiment 27. A population of oligomeric compounds of any of embodiments 1-9 and 13-24, or modified oligonucleotides of any of embodiments 10-12, wherein all of the phosphorothioate internucleoside linkages of the modified oligonucleotide are stereorandom.

Embodiment 28. An oligomeric compound comprising a modified oligonucleotide according to the following chemical notation: TₖₛTₖₛ^{m}Cₖₛ^{m}C_{ds}C_{ys}G_{ds}A_{ds}A_{ds}T_{ds}G_{ds}T_{ds}^{m}C_{ds}^{m}C_{ds}GₖₛAₖₛ^{m}Cₖ (SEQ ID NO: 20), wherein:
A=an adenine nucleobase,
^{m}C=a 5-methylcytosine nucleobase,
C=a cytosine nucleobase,
G=a guanine nucleobase,
T=a thymine nucleobase,
y=a 2′-OMe sugar moiety,
k=a cEt sugar moiety,
d=a 2′-β-D-deoxyribosyl sugar moiety, and
s=a phosphorothioate internucleoside linkage.

Embodiment 29. The oligomeric compound of embodiment 28 comprising a conjugate group.

Embodiment 30. The oligomeric compound of embodiment 29, wherein the conjugate group comprises a conjugate moiety and a conjugate linker.

Embodiment 31. The oligomeric compound of embodiment 29, wherein the conjugate group comprises C₁₀-C₂₄ alkyl.

Embodiment 32. The oligomeric compound of embodiment 29, wherein the conjugate group comprises C₁₆ alkyl.

Embodiment 33. The oligomeric compound of embodiment 30, wherein the conjugate moiety is a cell-targeting moiety.

Embodiment 34. The oligomeric compound of embodiment 30, wherein the cell-targeting moiety is selected from a carbohydrate, an antibody, and an antibody fragment.

Embodiment 35. The oligomeric compound of any of embodiments 33-34, wherein the cell-targeting moiety binds a cell surface receptor on a skeletal muscle cell.

Embodiment 36. The oligomeric compound of any of embodiments 33-35, wherein the cell-targeting moiety is an antibody or an antibody fragment that binds a transferrin receptor.

Embodiment 37. A modified oligonucleotide according to the following chemical structure:

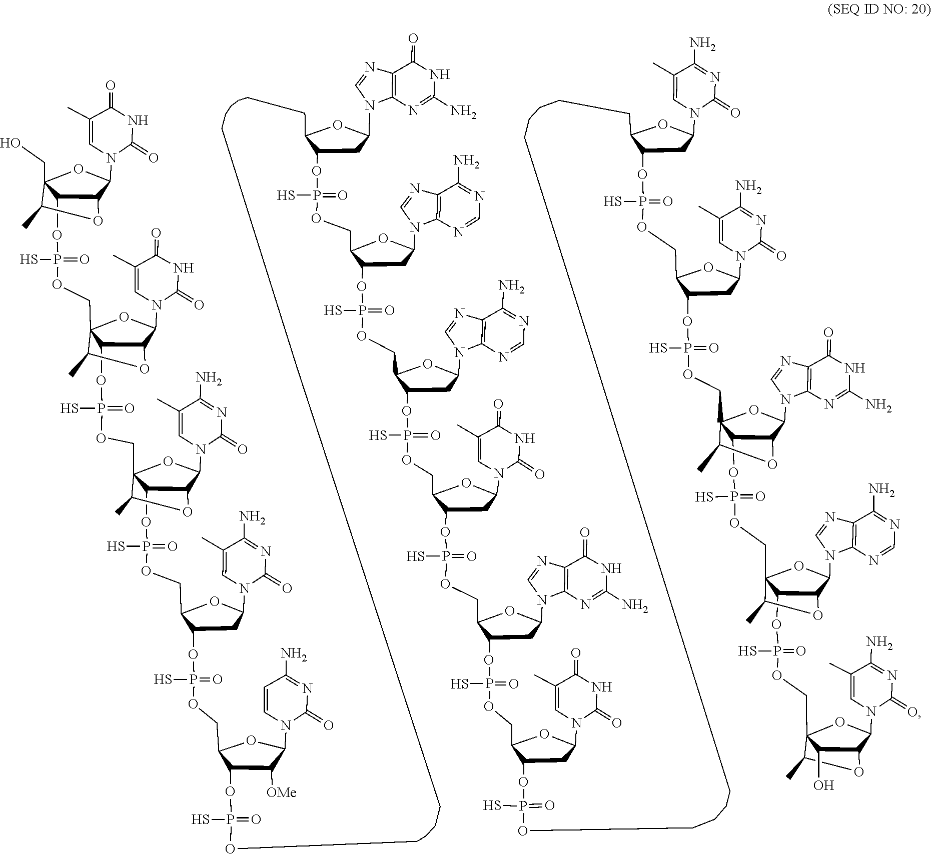

or a salt thereof.

Embodiment 38. The modified oligonucleotide of embodiment 37, which is a sodium salt or a potassium salt.

Embodiment 39. A modified oligonucleotide according to the following chemical structure:

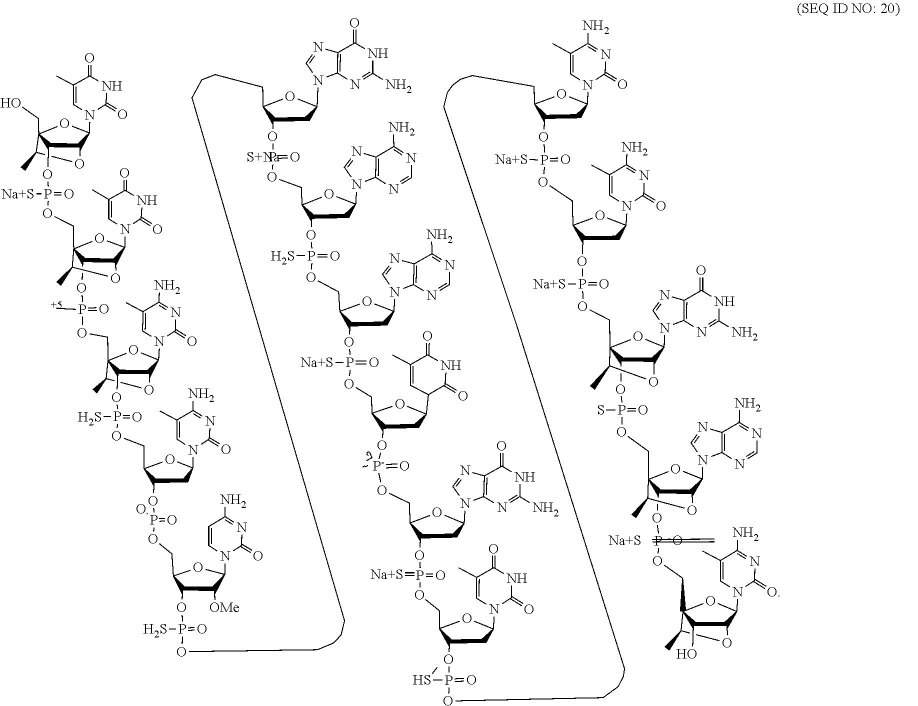

Embodiment 40. An oligomeric compound according to the following chemical structure:

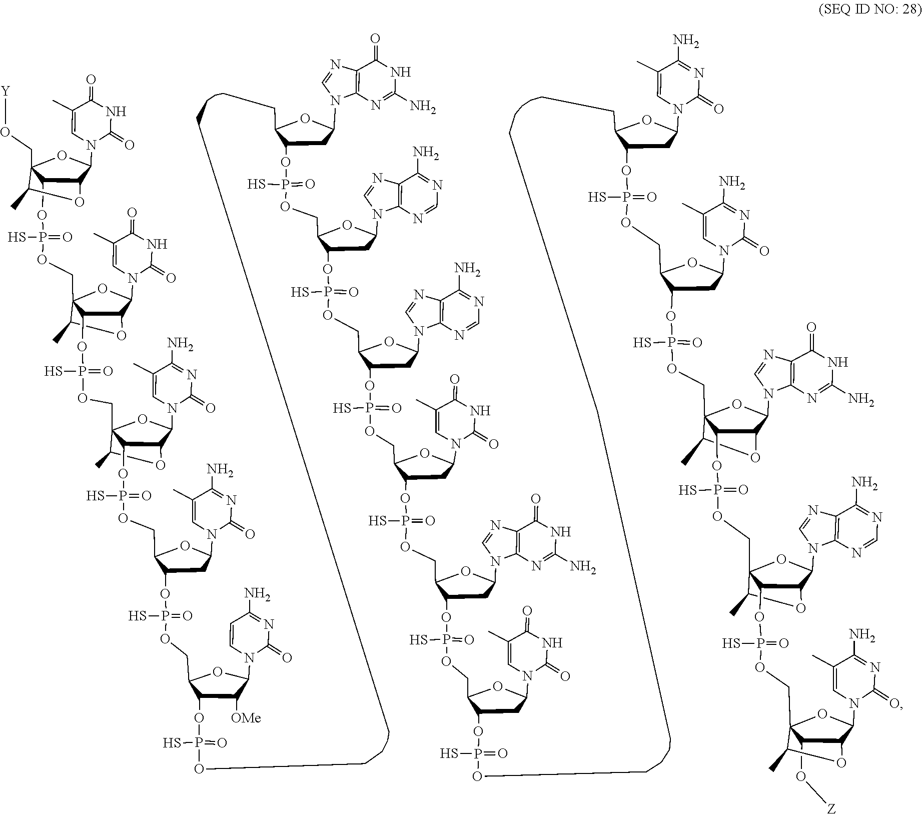

or a salt thereof, wherein Y and Z are selected from H and a conjugate group, wherein at least one of Y and Z is a conjugate group.

Embodiment 41. The oligomeric compound of embodiment 40, which is a sodium salt or a potassium salt.

Embodiment 42. An oligomeric compound according to the following chemical structure:

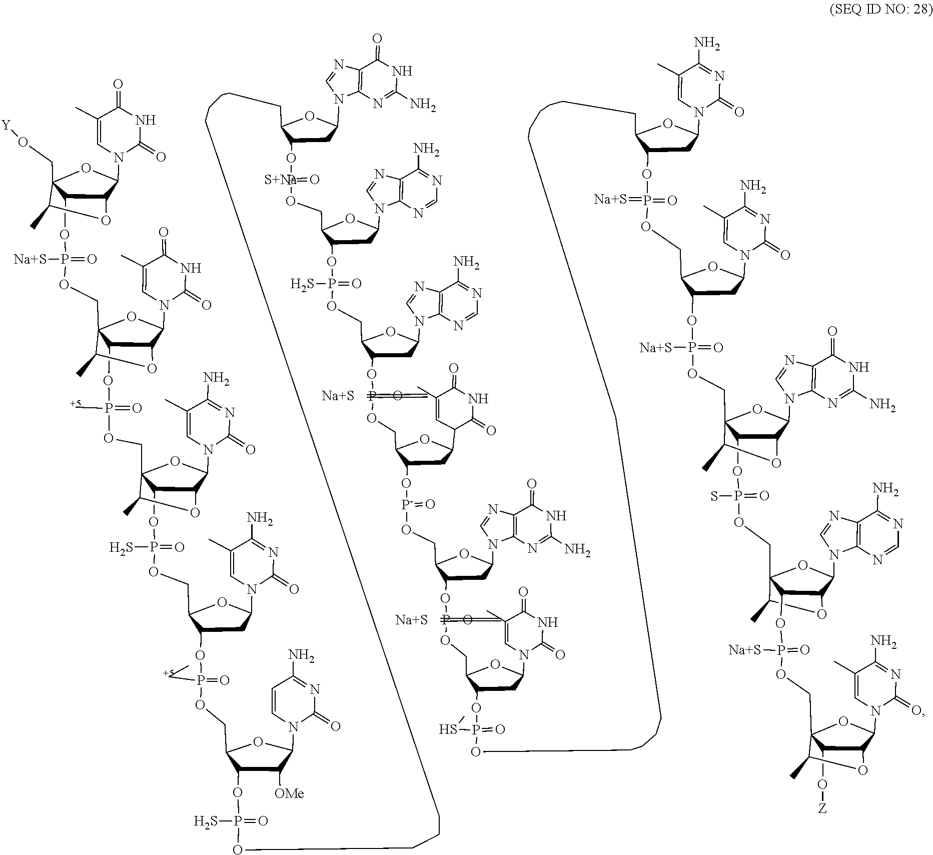

wherein Y and Z are selected from H and a conjugate group, wherein at least one of Y and Z is a conjugate group.

Embodiment 43. The oligomeric compound of any of embodiments 40-42, wherein the conjugate group comprises a conjugate moiety and a conjugate linker.

Embodiment 44. The oligomeric compound of any of embodiments 40-42, wherein the conjugate group comprises C₁₀-C₂₄ alkyl.

Embodiment 45. The oligomeric compound of any of embodiments 40-42, wherein the conjugate group comprises C₁₆.

Embodiment 46. The oligomeric compound of embodiment 43, wherein the conjugate moiety is a cell-targeting moiety.

Embodiment 47. The oligomeric compound of embodiment 46, wherein the cell-targeting moiety binds a cell surface receptor on a skeletal muscle cell.

Embodiment 48. The oligomeric compound of embodiment 46 or embodiment 47, wherein the cell-targeting moiety is selected from a carbohydrate and an antibody.

Embodiment 49. The oligomeric compound of embodiment 46 or embodiment 47, wherein the cell-targeting moiety is an antibody or an antibody fragment that binds a transferrin receptor.

Embodiment 50. The oligomeric compound of embodiment 46, wherein the cell-targeting moiety comprises a GalNAc.

Embodiment 51. The oligomeric compound of any of embodiments 40-42, wherein Y is:

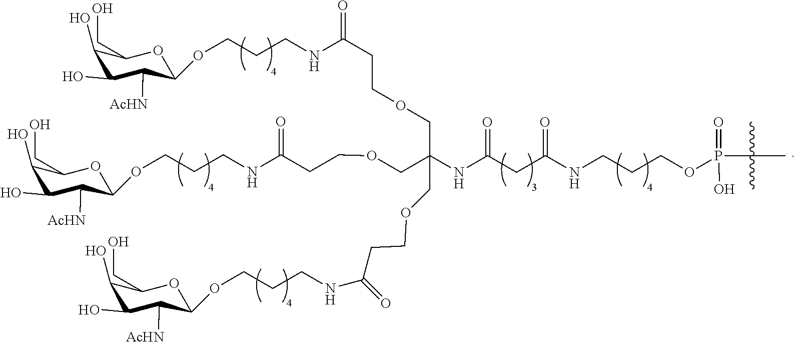

Embodiment 52. A prodrug of the oligomeric compound of any of embodiments 28-36 and 40-51 or the modified oligonucleotide of any of embodiments 37-39.

Embodiment 53. The oligomeric compound any of embodiments 28-36 and 40-51, wherein the oligomeric compound is a prodrug.

Embodiment 54. A population of oligomeric compounds of any of embodiments 28-36 and 40-51 or modified oligonucleotides of any of embodiments 37-39, wherein all of the phosphorothioate internucleoside linkages of the modified oligonucleotide are stereorandom.

Embodiment 55. An oligomeric compound comprising a modified oligonucleotide according to the following chemical notation: ^{m}CₖₛGₖₒAₖₒA_{ds}U_{ys}G_{ds}T_{ds}^{m}C_{ds}^{m}C_{ds}G_{ds}A_{ds}^{m}C_{ds}A_{ds}GₖₒTₖₛGₖ (SEQ ID NO: 14), wherein:
A=an adenine nucleobase,
^{m}C=a 5-methylcytosine nucleobase,
G=a guanine nucleobase,
T=a thymine nucleobase,
U=a uracil nucleobase,
y=a 2′-OMe sugar moiety,
k=a cEt sugar moiety,
d=a 2′-β-D-deoxyribosyl sugar moiety,
s=a phosphorothioate internucleoside linkage, and
o=a phosphodiester internucleoside linkage.

Embodiment 56. The oligomeric compound of embodiment 55 comprising a conjugate group.

Embodiment 57. The oligomeric compound of embodiment 56, wherein the conjugate group comprises a conjugate moiety and a conjugate linker.

Embodiment 58. The oligomeric compound of embodiment 56, wherein the conjugate group comprises C₁₀-C₂₄ alkyl.

Embodiment 59. The oligomeric compound of embodiment 56, wherein the conjugate group comprises C₁₆ alkyl.

Embodiment 60. The oligomeric compound of embodiment 57, wherein the conjugate moiety is a cell-targeting moiety.

Embodiment 61. The oligomeric compound of embodiment 60, wherein the cell-targeting moiety is selected from a carbohydrate, an antibody, and an antibody fragment.

Embodiment 62. The oligomeric compound of any of embodiments 60-61, wherein the cell-targeting moiety binds a cell surface receptor on a skeletal muscle cell.

Embodiment 63. The oligomeric compound of any of embodiments 60-62, wherein the cell-targeting moiety is an antibody or an antibody fragment that binds a transferrin receptor.

Embodiment 64. A modified oligonucleotide according to the following chemical structure:

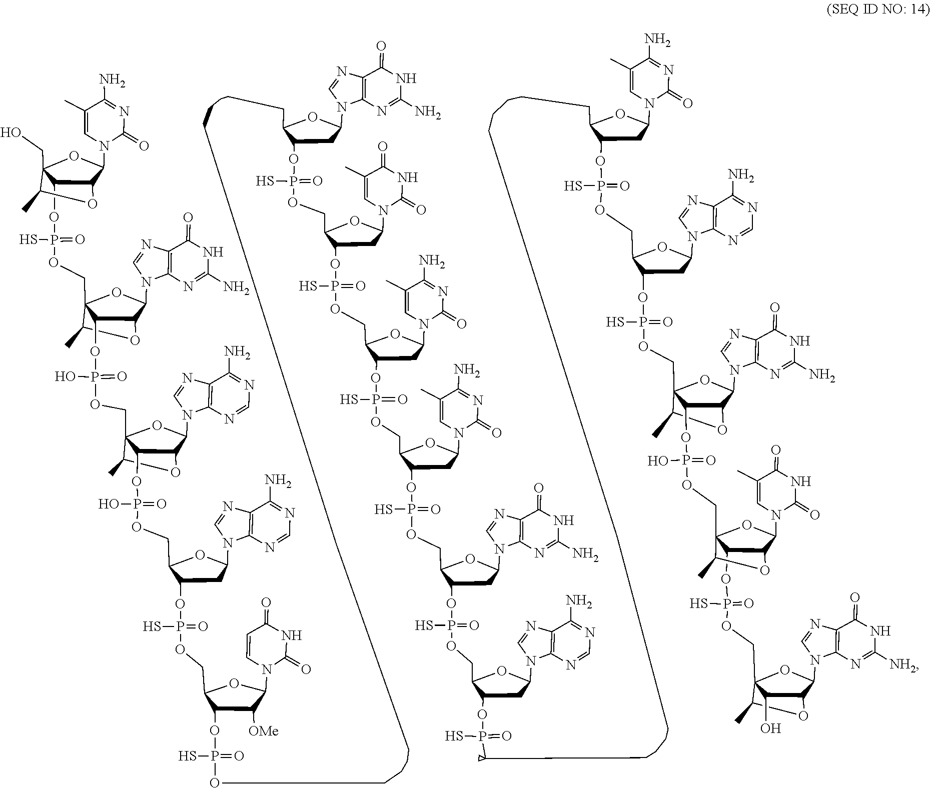

or a salt thereof.

Embodiment 65. The modified oligonucleotide of embodiment 64, which is a sodium salt or a potassium salt.

Embodiment 66. A modified oligonucleotide according to the following chemical structure:

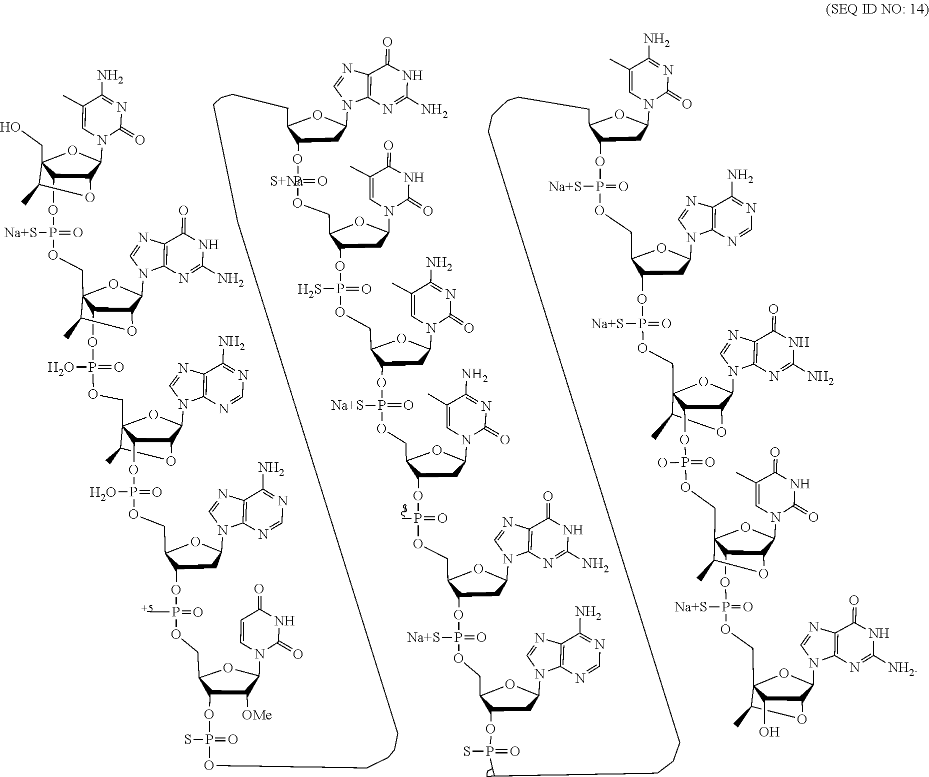

Embodiment 67. An oligomeric compound according to the following chemical structure:

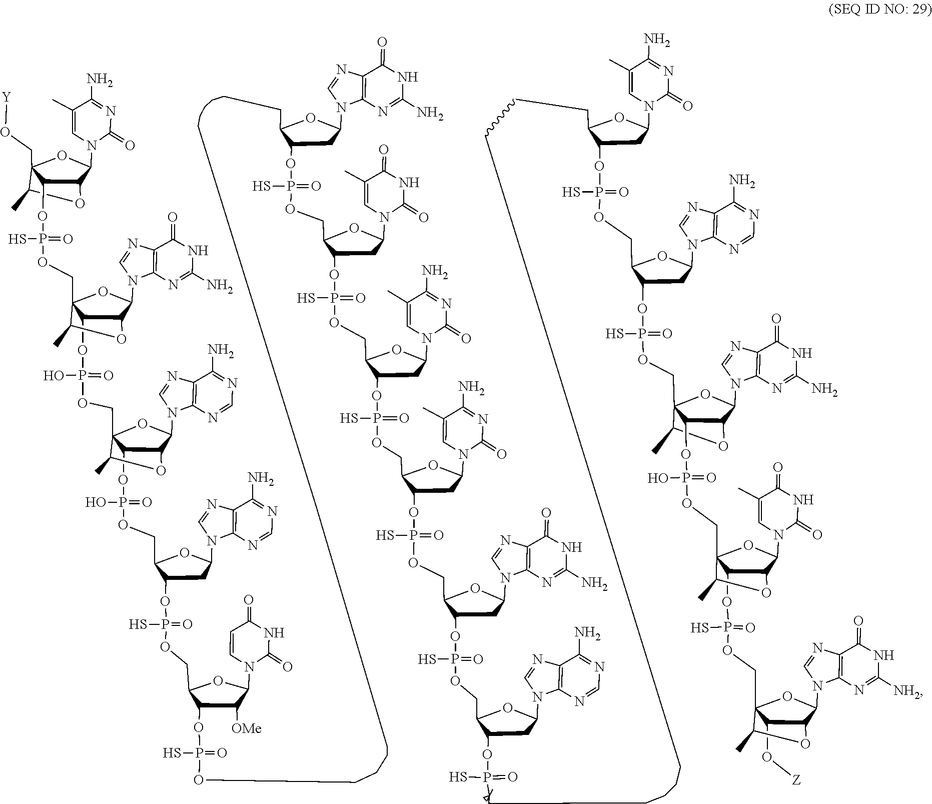

or a salt thereof, wherein Y and Z are selected from H and a conjugate group, wherein at least one of Y and Z is a conjugate group.

Embodiment 68. The oligomeric compound of embodiment 67, which is a sodium salt or a potassium salt.

Embodiment 69. An oligomeric compound according to the following chemical structure:

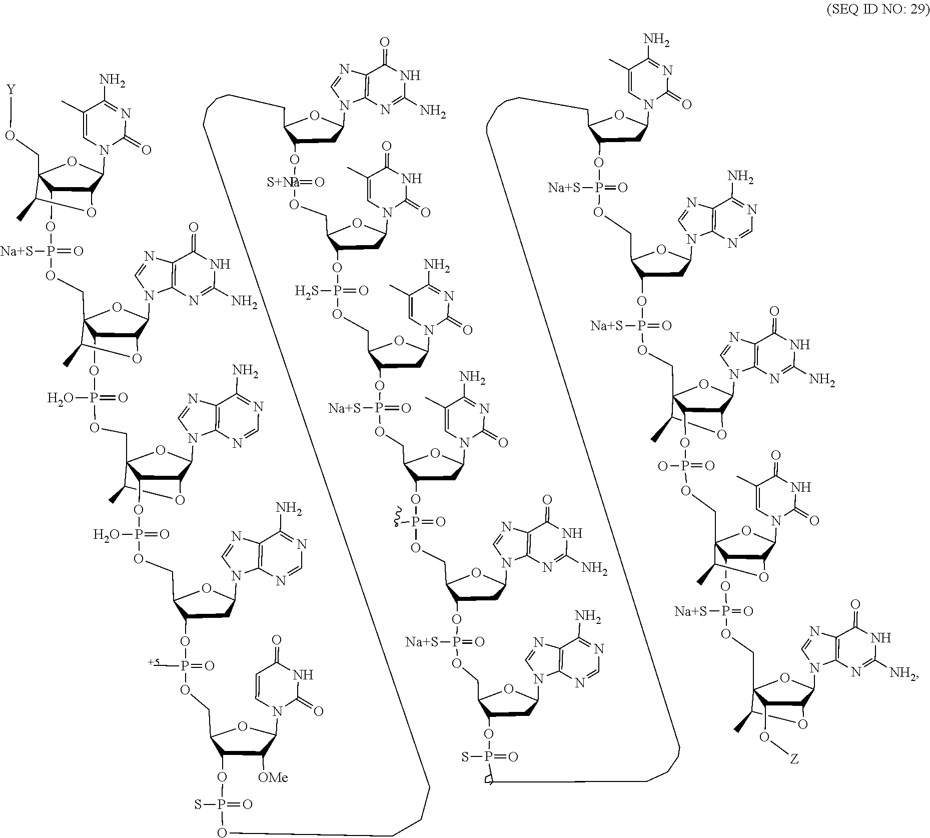

wherein Y and Z are selected from H and a conjugate group, wherein at least one of Y and Z is a conjugate group.

Embodiment 70. The oligomeric compound of any of embodiments 67-69, wherein the conjugate group comprises a conjugate moiety and a conjugate linker.

Embodiment 71. The oligomeric compound of any of embodiments 67-69, wherein the conjugate moiety is C₁₀-C₂₄ alkyl.

Embodiment 72. The oligomeric compound of any of embodiments 67-69, wherein the conjugate moiety is C₁₆.

Embodiment 73. The oligomeric compound of embodiment 70, wherein the conjugate moiety is a cell-targeting moiety.

Embodiment 74. The oligomeric compound of embodiment 73, wherein the cell-targeting moiety binds a cell surface receptor on a skeletal muscle cell.

Embodiment 75. The oligomeric compound of any of embodiments 73-74, wherein the cell-targeting moiety is selected from a carbohydrate and an antibody.

Embodiment 76. The oligomeric compound of any of embodiments 73-74, wherein the cell-targeting moiety is an antibody or an antibody fragment that binds a transferrin receptor.

Embodiment 77. The oligomeric compound of embodiment 73, wherein the cell-targeting moiety comprises a GalNAc.

Embodiment 78. The oligomeric compound of any of embodiments 67-69, wherein Y is:

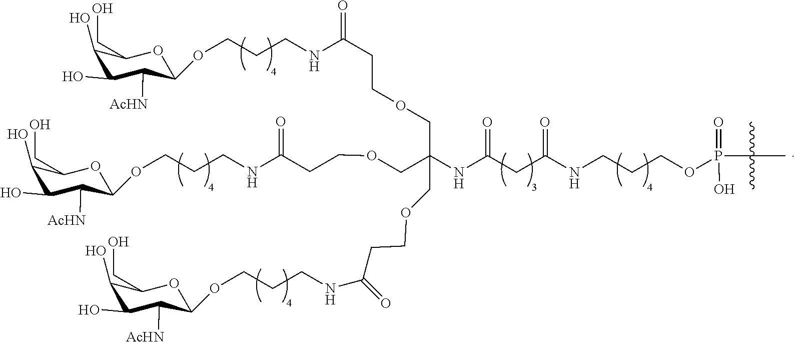

Embodiment 79. A prodrug of the oligomeric compound of any of embodiments 55-63 and 67-78 or the modified oligonucleotide of any of embodiments 64-66.

Embodiment 80. The oligomeric compound of any of embodiments 55-63 and 67-78, wherein the oligomeric compound is a prodrug.

Embodiment 81. A population of oligomeric compounds of any of embodiments 55-63 and 67-78 or modified oligonucleotides of any of embodiments 64-66, wherein all of the phosphorothioate internucleoside linkages of the modified oligonucleotide are stereorandom.

Embodiment 82. An oligomeric compound comprising a modified oligonucleotide according to the following chemical notation: ^{m}CₖₛTₖₒTₑₒT_{ds}T_{ds}A_{ds}T_{ds}T_{ds}^{m}C_{ds}G_{ds}^{m}C_{ds}G_{ds}A_{ds}GₖₒGₖₛGₖ (SEQ ID NO: 15), wherein:
A=an adenine nucleobase,
^{m}C=a 5-methylcytosine nucleobase,
G=a guanine nucleobase,
T=a thymine nucleobase,
k=a cEt sugar moiety,
e=a 2′-β-D-deoxyribosyl sugar moiety,
s=a phosphorothioate internucleoside linkage, and
o=a phosphodiester internucleoside linkage.

Embodiment 83. The oligomeric compound of embodiment 82 comprising a conjugate group.

Embodiment 84. The oligomeric compound of embodiment 83, wherein the conjugate group comprises a conjugate moiety and a conjugate linker.

Embodiment 85. The oligomeric compound of embodiment 83, wherein the conjugate group comprises C₁₀-C₂₄ alkyl.

Embodiment 86. The oligomeric compound of embodiment 83, wherein the conjugate group comprises C₁₆ alkyl.

Embodiment 87. The oligomeric compound of embodiment 84, wherein the conjugate moiety is a cell-targeting moiety.

Embodiment 88. The oligomeric compound of embodiment 87, wherein the cell-targeting moiety is selected from a carbohydrate, an antibody, and an antibody fragment.

Embodiment 89. The oligomeric compound of any of embodiments 87-88, wherein the cell-targeting moiety binds a cell surface receptor on a skeletal muscle cell.

Embodiment 90. The oligomeric compound of any of embodiments 87-89, wherein the cell-targeting moiety is an antibody or an antibody fragment that binds a transferrin receptor.

Embodiment 91. A modified oligo nucleotide according to the following chemical structure:

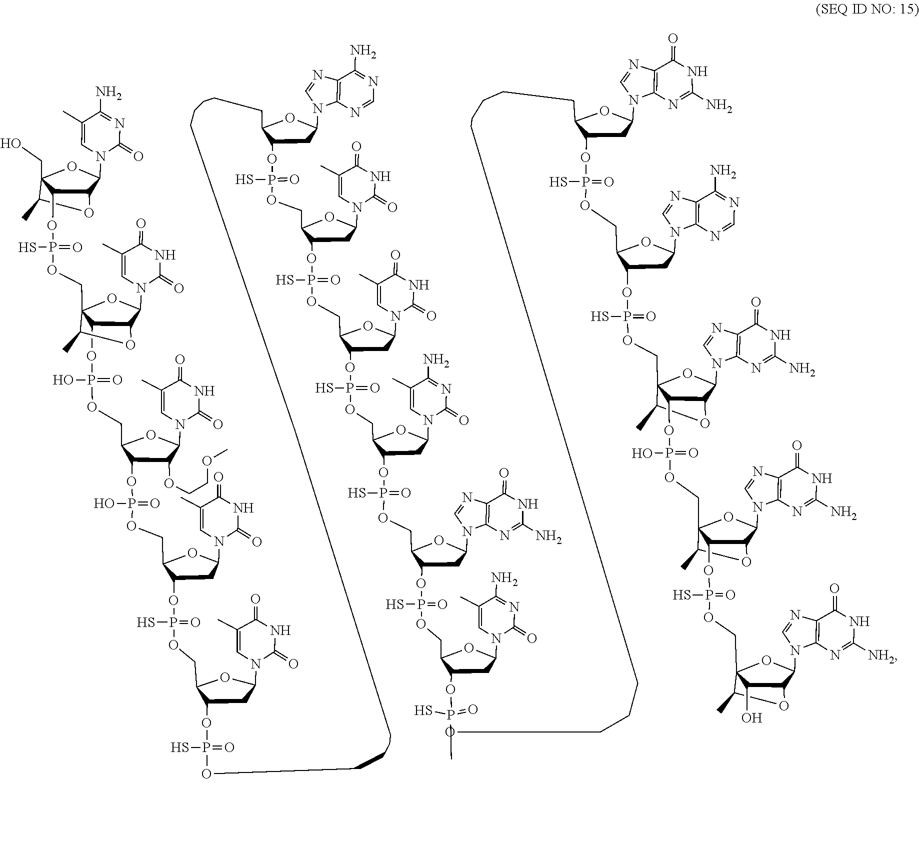

or a salt thereof.

Embodiment 92. The modified oligonucleotide of embodiment 91, which is a sodium salt or a potassium salt.

Embodiment 93. A modified oligonucleotide according to the following chemical structure:

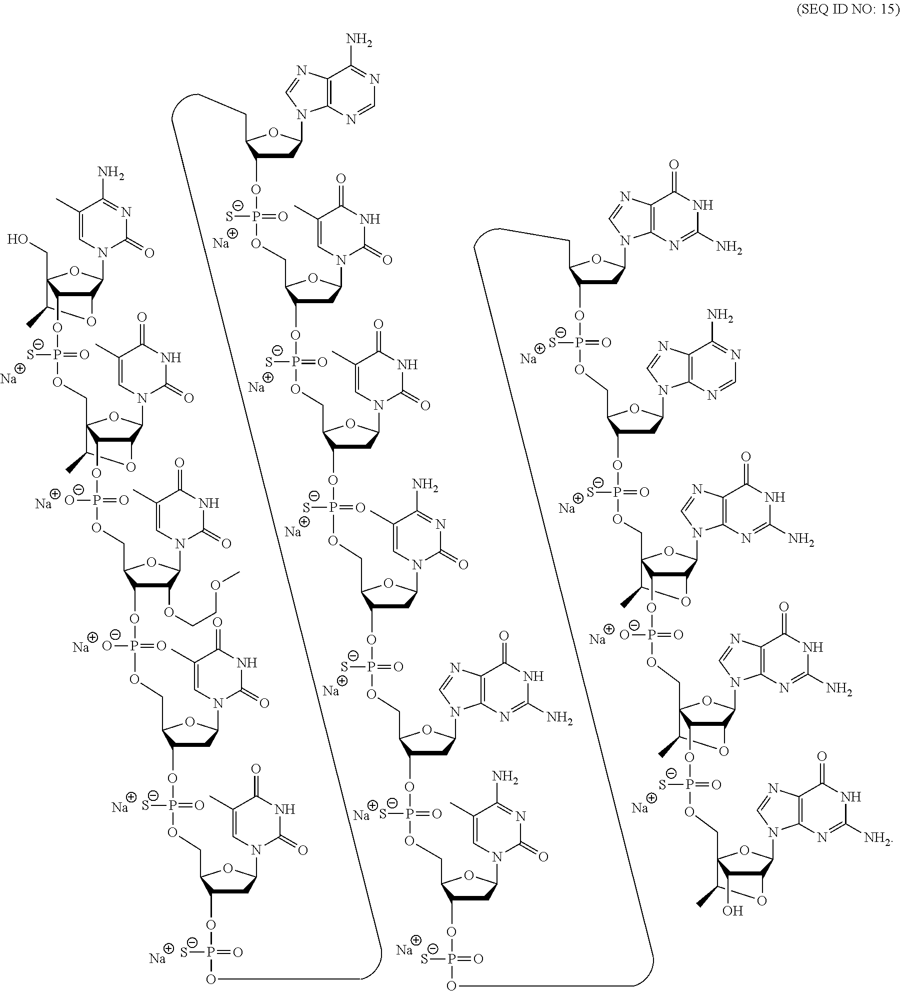

Embodiment 94. An oligomeric compound according to the following chemical structure:

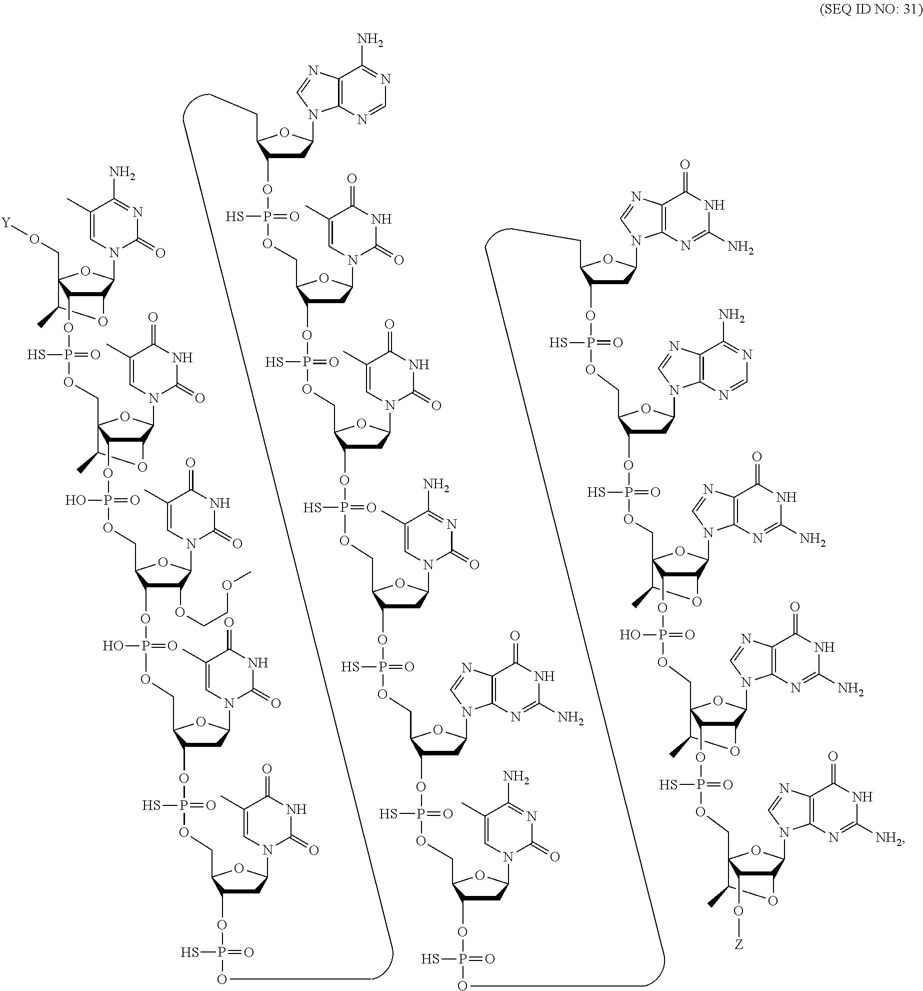

or a salt thereof, wherein Y and Z are selected from H and a conjugate group, wherein at least one of Y and Z is a conjugate group.

Embodiment 95. The oligomeric compound of embodiment 94, which is a sodium salt or a potassium salt.

Embodiment 96. An oligomeric compound according to the following chemical structure:

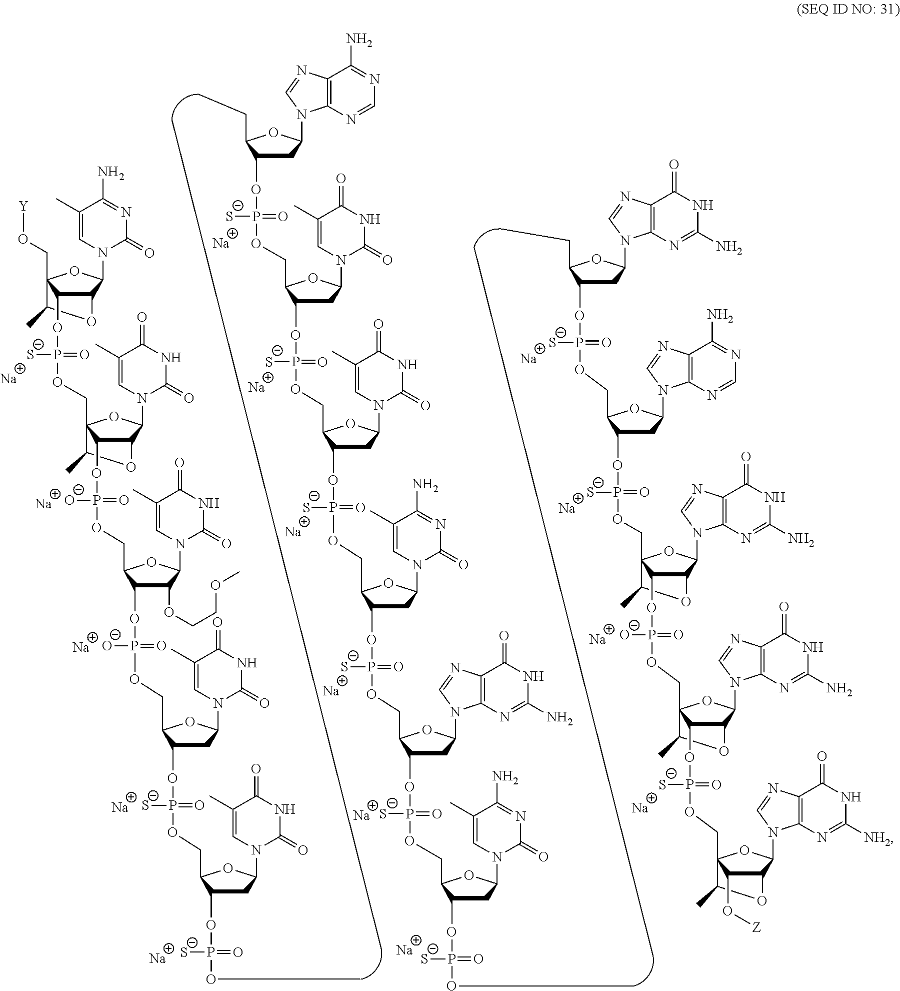

wherein Y and Z are selected from H and a conjugate group, wherein at least one of Y and Z is a conjugate group.

Embodiment 97. The oligomeric compound of any of embodiments 94-96, wherein the conjugate group comprises a conjugate moiety and a conjugate linker.

Embodiment 98. The oligomeric compound of any of embodiments 94-96, wherein the conjugate group comprises C₁₀-C₂₄ alkyl.

Embodiment 99. The oligomeric compound of any of embodiments 94-96, wherein the conjugate group comprises C₁₆.

Embodiment 100. The oligomeric compound of embodiment 97, wherein the conjugate moiety is a cell-targeting moiety.

Embodiment 101. The oligomeric compound of embodiment 100, wherein the cell-targeting moiety binds a cell surface receptor on a skeletal muscle cell.

Embodiment 102. The oligomeric compound of any of embodiments 100-101, wherein the cell-targeting moiety is selected from a carbohydrate and an antibody.

Embodiment 103. The oligomeric compound of any of embodiments 100-101, wherein the cell-targeting moiety is an antibody or an antibody fragment that binds a transferrin receptor.

Embodiment 104. The oligomeric compound of embodiment 100, wherein the cell-targeting moiety comprises a GalNAc.

Embodiment 105. The oligomeric compound of any of embodiments 94-96, wherein Y is:

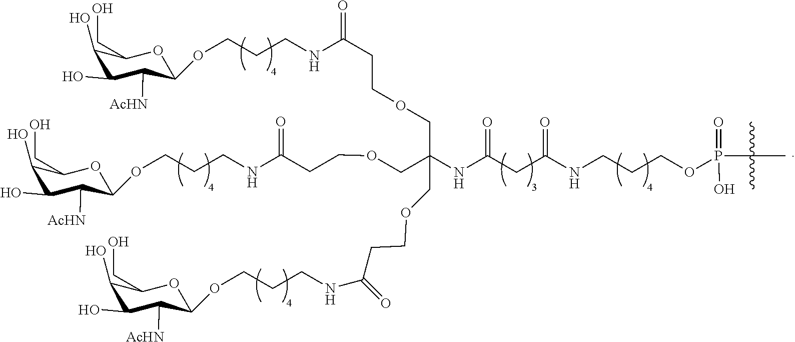

Embodiment 106. A prodrug of the oligomeric compound of any of embodiments 82-90 and 94-105 or the modified oligonucleotide of any of embodiments 91-93.

Embodiment 107. The oligomeric compound of any of embodiments 82-90 and 94-105, wherein the oligomeric compound is a prodrug.

Embodiment 108. A population of oligomeric compounds of any of embodiments 82-90 and 94-105 or modified oligonucleotides of any of embodiments 91-93, wherein all of the phosphorothioate internucleoside linkages of the modified oligonucleotide are stereorandom.

Embodiment 109. An oligomeric compound comprising a modified oligonucleotide according to the following chemical notation: Aₖₛ^{m}CₖₒAₖₒA_{ds}T_{ds}A_{ds}A_{ds}T_{ds}A_{ds}^{m}C_{ds}^{m}C_{ds}G_{ds}AₖₒGₖₛGₖ (SEQ ID NO: 11), wherein:
A=an adenine nucleobase,
^{m}C=a 5-methylcytosine nucleobase,
G=a guanine nucleobase,
T=a thymine nucleobase,
k=a cEt sugar moiety,
d=a 2′-β-D-deoxyribosyl sugar moiety,
s=a phosphorothioate internucleoside linkage, and
o=a phosphodiester internucleoside linkage.

Embodiment 110. The oligomeric compound of embodiment 109 comprising a conjugate group.

Embodiment 111. The oligomeric compound of embodiment 110, wherein the conjugate group comprises a conjugate moiety and a conjugate linker.

Embodiment 112. The oligomeric compound of embodiment 110, wherein the conjugate group comprises C₁₀-C₂₄ alkyl.

Embodiment 113. The oligomeric compound of embodiment 110, wherein the conjugate group comprises C₁₆ alkyl.

Embodiment 114. The oligomeric compound of embodiment 111, wherein the conjugate moiety is a cell-targeting moiety.

Embodiment 115. The oligomeric compound of embodiment 114, wherein the cell-targeting moiety is selected from a carbohydrate, an antibody, and an antibody fragment.

Embodiment 116. The oligomeric compound of any of embodiments 114-115, wherein the cell-targeting moiety binds a cell surface receptor on a skeletal muscle cell.

Embodiment 117. The oligomeric compound of any of embodiments 114-116, wherein the cell-targeting moiety is an antibody or an antibody fragment that binds a transferrin receptor.

Embodiment 118. A modified oligonucleotide according to the following chemical structure:

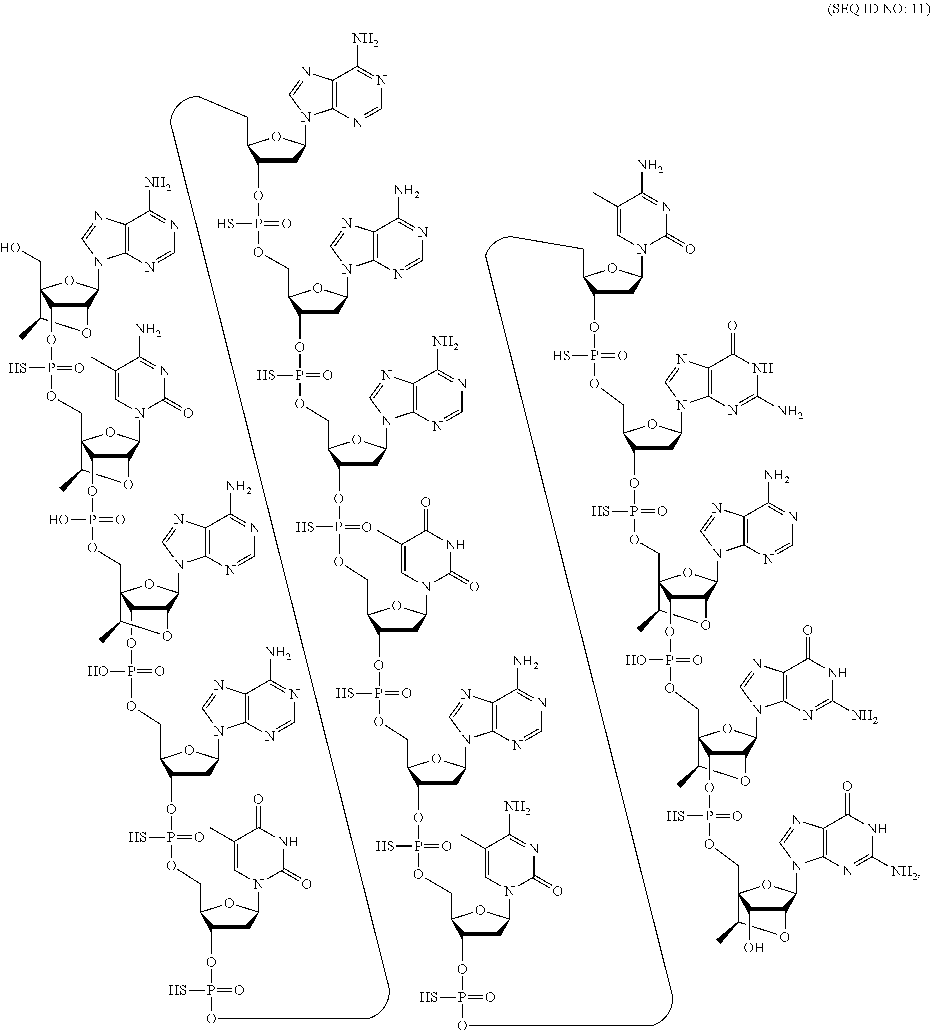

or a salt thereof.

Embodiment 119. The modified oligonucleotide of embodiment 118, which is a sodium salt or a potassium salt.

Embodiment 120. A modified oligonucleotide according to the following chemical structure:

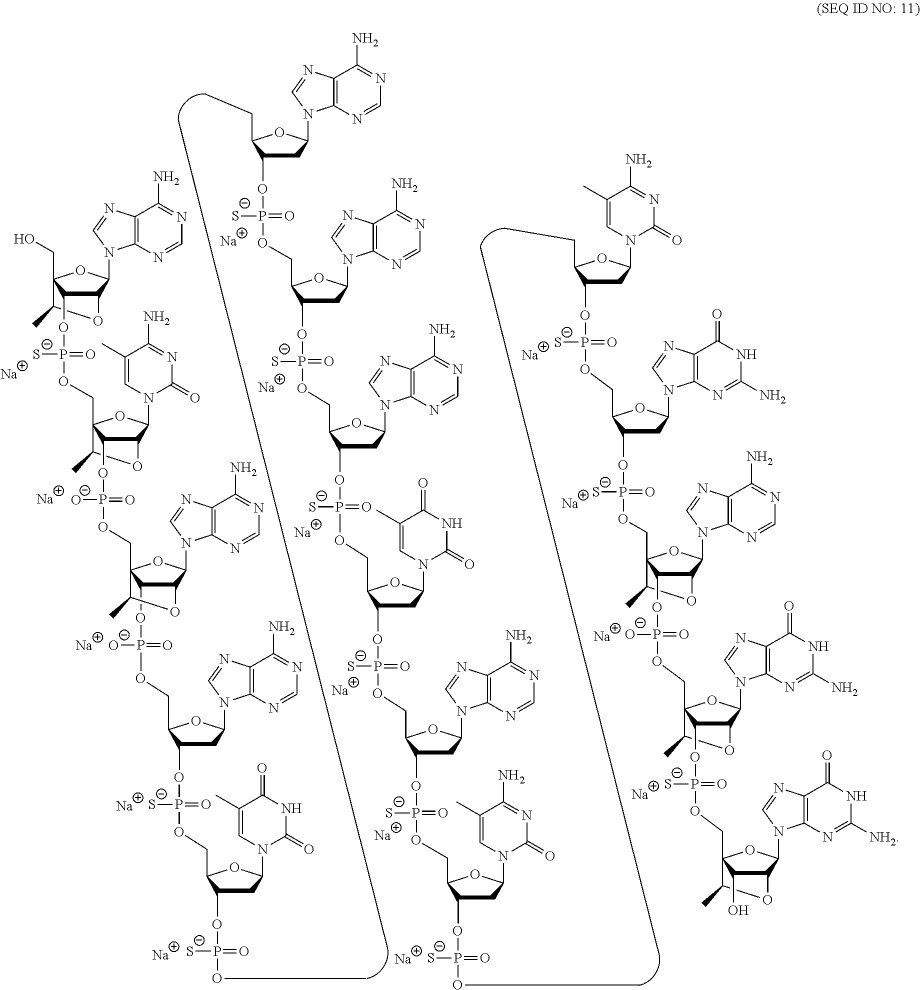

Embodiment 121. An oligomeric compound according to the following chemical structure:

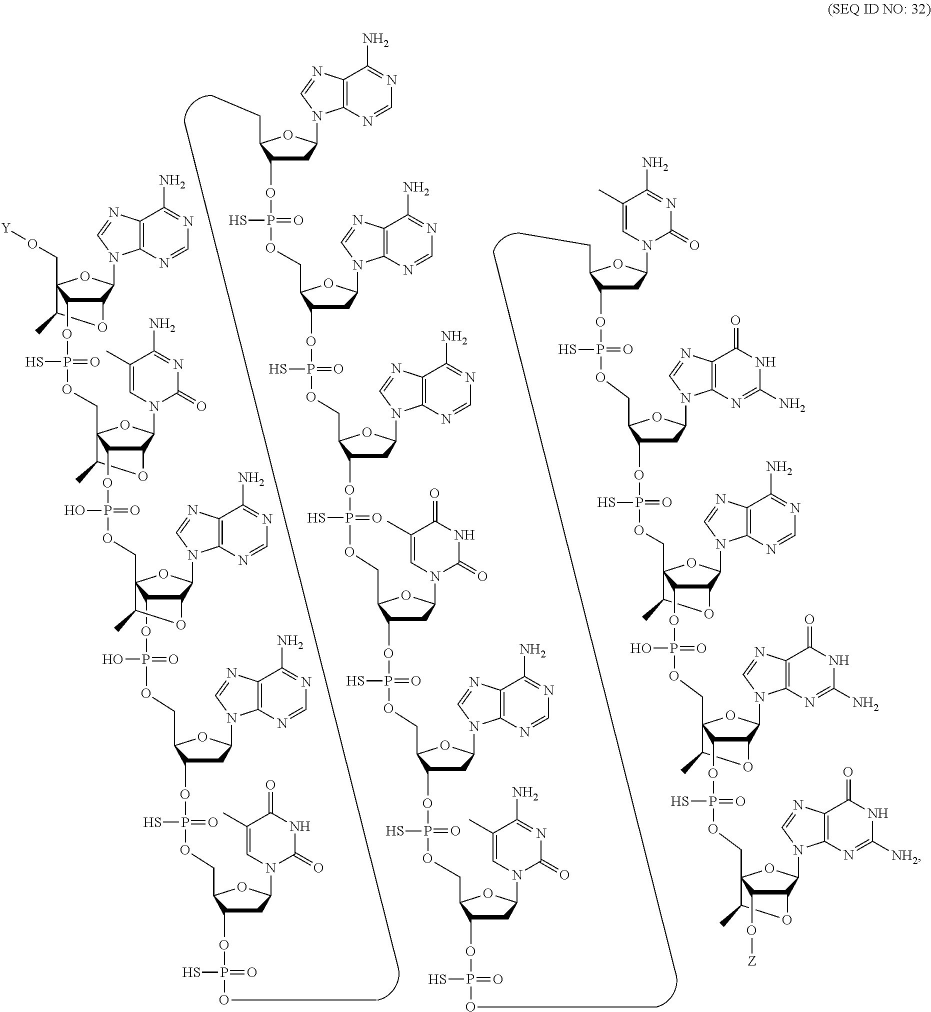

or a salt thereof, wherein Y and Z are selected from II and a conjugate group, wherein at least one of Y and Z is a conjugate group.

Embodiment 122. The oligomeric compound of embodiment 121. which is a sodium salt or a potassium salt.

Embodiment 123. An oligomeric compound according to the following chemical structure:

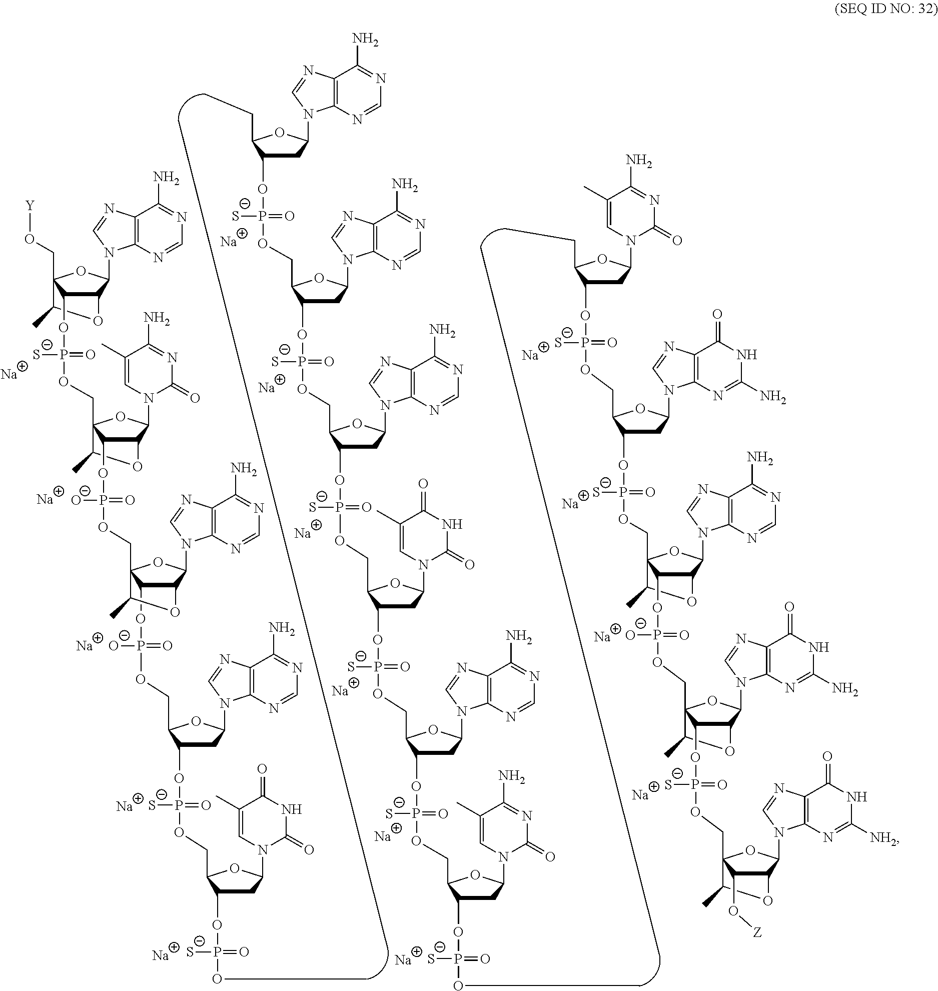

wherein Y and Z are selected from H and a conjugate group, wherein at least one of Y and Z is a conjugate group.

Embodiment 124. The oligomeric compound of any of embodiments 121-123, wherein the conjugate group comprises a conjugate moiety and a conjugate linker.

Embodiment 125. The oligomeric compound of any of embodiments 121-123, wherein the conjugate group comprises C₁₀-C₂₄ alkyl.

Embodiment 126. The oligomeric compound of any of embodiments 121-123, wherein the conjugate group comprises C₁₆.

Embodiment 127. The oligomeric compound of embodiment 124, wherein the conjugate moiety is a cell-targeting moiety.

Embodiment 128. The oligomeric compound of embodiment 127, wherein the cell-targeting moiety binds a cell surface receptor on a skeletal muscle cell.

Embodiment 129. The oligomeric compound of embodiment 127 or embodiment 128, wherein the cell-targeting moiety is selected from a carbohydrate and an antibody.

Embodiment 130. The oligomeric compound of embodiment 127 or embodiment 128, wherein the cell-targeting moiety is an antibody or an antibody fragment that binds a transferrin receptor.

Embodiment 131. The oligomeric compound of embodiment 127, wherein the cell-targeting moiety comprises a GalNAc.

Embodiment 132. The oligomeric compound of any of embodiments 121-123, wherein Y is:

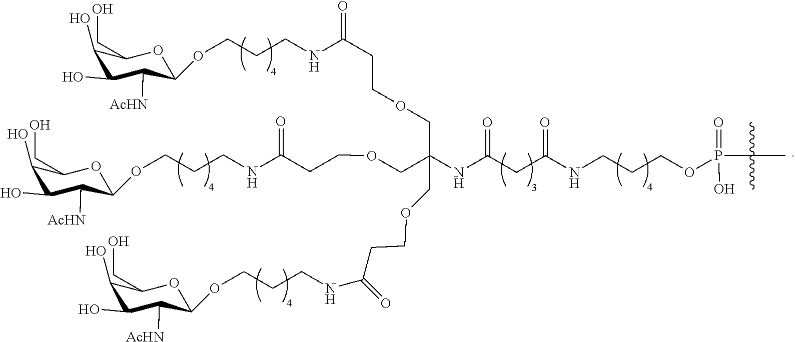

Embodiment 133. A prodrug of the oligomeric compound of any of embodiments 109-117 and 121-132 or the modified oligonucleotide of any of embodiments 118-120.

Embodiment 134. The oligomeric compound of any of embodiments 109-117 and 121-132, wherein the oligomeric compound is a prodrug.

Embodiment 135. A population of oligomeric compounds of any of embodiments 109-117 and 121-132 or modified oligonucleotides of any of embodiments 118-120, wherein all of the phosphorothioate internucleoside linkages of the modified oligonucleotide are stereorandom.

Embodiment 136. A pharmaceutical composition an oligomeric compound of any of embodiments 1-9, 13-24, 26, 28-36, 40-51, 53, 55-63, 67-78, 80, 82-90, 94-105, 107, 109-117, 121-132, or 134, a modified oligonucleotide of any of embodiments 10-12, 37-39, 64-66, 91-93, 118-120, a prodrug of any of embodiments 25, 52, 79, 106, or133, or a population of oligomeric compounds of any of embodiments 27, 54, 81, 108, or 135, and a pharmaceutically acceptable diluent.

Embodiment 137. The pharmaceutical composition of embodiment 136, wherein the pharmaceutically acceptable diluent is water or phosphate-buffered saline.

Embodiment 138. The pharmaceutical composition of embodiment 137, wherein the pharmaceutical composition consists essentially of the oligomeric compound, the modified oligonucleotide, the prodrug, or the population, and water or phosphate-buffered saline.

Embodiment 139. A method comprising administering to a subject an oligomeric compound of any of embodiments 1-9, 13-24, 26, 28-36, 40-51, 53, 55-63, 67-78, 80, 82-90, 94-105, 107, 109-117, 121-132, or 134, a modified oligonucleotide of any of embodiments 10-12, 37-39, 64-66, 91-93, 118-120, a prodrug of any of embodiments 25, 52, 79, 106, or133, or a population of oligomeric compounds of any of embodiments 27, 54, 81, 108, or 135, or a pharmaceutical composition of any of embodiments 136-138.

Embodiment 140. A method of treating a disease associated with DMPK, comprising administering to a subject having a disease associated with DMPK a therapeutically effective amount of an oligomeric compound of any of embodiments 1-9, 13-24, 26, 28-36, 40-51, 53, 55-63, 67-78, 80, 82-90, 94-105, 107, 109-117, 121-132, or 134, a modified oligonucleotide of any of embodiments 10-12, 37-39, 64-66, 91-93, 118-120, a prodrug of any of embodiments 25, 52, 79, 106, or133, or a population of oligomeric compounds of any of embodiments 27, 54, 81, 108, or 135, or a pharmaceutical composition of any of embodiments 136-138: thereby treating the disease associated with DMPK.

Embodiment 141. The method of embodiment 140, wherein the disease associated with DMPK is type 1 myotonic dystrophy.

Embodiment 142. The method of any of embodiments 140-141, wherein the administering an oligomeric compound of any of embodiments 1-9, 13-24, 26, 28-36, 40-51, 53, 55-63, 67-78, 80, 82-90, 94-105, 107, 109-117, 121-132, or 134, a modified oligonucleotide of any of embodiments 10-12, 37-39, 64-66, 91-93, 118-120, a prodrug of any of embodiments 25, 52, 79, 106, or 133, or a population of oligomeric compounds of any of embodiments 27, 54, 81, 108, or 135, or a pharmaceutical composition of any of embodiments 136-138 reduces myotonia and/or spliceopathy in the subject.

Embodiment 143, The method of any of embodiments 139-142, wherein the subject is human.

Embodiment 144. A method of reducing expression of DMPK in a cell, comprising contacting the cell with an oligomeric compound of any of embodiments 1-9, 13-24, 26, 28-36, 40-51, 53, 55-63, 67-78, 80, 82-90, 94-105, 107, 109-117, 121-132, or 134, a modified oligonucleotide of any of embodiments 10-12, 37-39, 64-66, 91-93, 118-120, a prodrug of any of embodiments 25, 52, 79, 106, or133, or a population of oligomeric compounds of any of embodiments 27, 54, 81, 108, or 135, or a pharmaceutical composition of any of embodiments 136-138.

Embodiment 145. The method of embodiment 144, wherein the cell is a muscle cell.

Embodiment 146. The method of embodiment 144 or 145, wherein the cell is a human cell.

Embodiment 147. Use of an oligomeric compound of any of embodiments 1-9, 13-24, 26, 28-36, 40-51, 53, 55-63, 67-78, 80, 82-90, 94-105, 107, 109-117, 121-132, or 134, a modified oligonucleotide of any of embodiments 10-12, 37-39, 64-66, 91-93, 118-120, a prodrug of any of embodiments 25, 52, 79, 106, or133, or a population of oligomeric compounds of any of embodiments 27, 54, 81, 108, or 135, or a pharmaceutical composition of any of embodiments 136-138 for treating a disease associated with DMPK.

Embodiment 148. Use of an oligomeric compound of any of embodiments 1-9, 13-24, 26, 28-36, 40-51, 53, 55-63, 67-78, 80, 82-90, 94-105, 107, 109-117, 121-132, or 134, a modified oligonucleotide of any of embodiments 10-12, 37-39, 64-66, 91-93, 118-120, a prodrug of any of embodiments 25, 52, 79, 106, or133, or a population of oligomeric compounds of any of embodiments 27, 54, 81, 108, or 135, or a pharmaceutical composition of any of embodiments 136-138 in the manufacture of a medicament for treating a disease associated with DMPK.

Embodiment 149. The use of any of embodiments 147-148, wherein the disease associated with DMPK is type 1 myotonic dystrophy.

### 1. Compound No. 1522461

In certain embodiments, Compound No. 1522461 is characterized as a mixed wing gapmer of linked nucleosides and having a nucleobase sequence (from 5′ to 3′) of TTCCCGAATGTCCGAC (SEQ ID NO 35), wherein each of nucleosides 1-3 and 14-16 (from 5′ to 3′) are cEt nucleosides, nucleoside 5 is a 2′-OMe nucleoside, and each of nucleosides 4 and 6-13 are 2′-β-D-deoxynucleosides, wherein the internucleoside linkages between nucleosides 2 to 3, 3 to 4 and 14 to 15 are phosphodiester internucleoside linkages, the internucleoside linkages between nucleosides 1 to 2, 4 to 5, 5 to 6, 6 to 7, 7 to 8, 8 to 9, 9 to 10, 10 to 11, 11 to 12, 12 to 13, 13 to 14, and 15 to 16 are phosphorothioate internucleoside linkages. The cytosines at positions 3, 4, 12, 13, and 16 are 5-methylcytosines, while the cytosine at position 5 is a non-methylated cytosine.

In certain embodiments, Compound No. 1522461 is represented by the following chemical notation: TₖₛTₖₒ^{m}Cₖₒ^{m}C_{ds}C_{ys}G_{ds}A_{ds}A_{ds}T_{ds}G_{ds}T_{ds}^{m}C_{ds}^{m}C_{ds}GₖₒAₖₛ^{m}Cₖ (SEQ ID NO: 13), wherein:
A=an adenine nucleobase,
mC=a 5-methylcytosine nucleobase,
C=a cytosine nucleobase,
G=a guanine nucleobase,
T=a thymine nucleobase,
y=a 2′-OMe sugar moiety,
k=a cEt sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1522461 is represented by the following chemical structure:

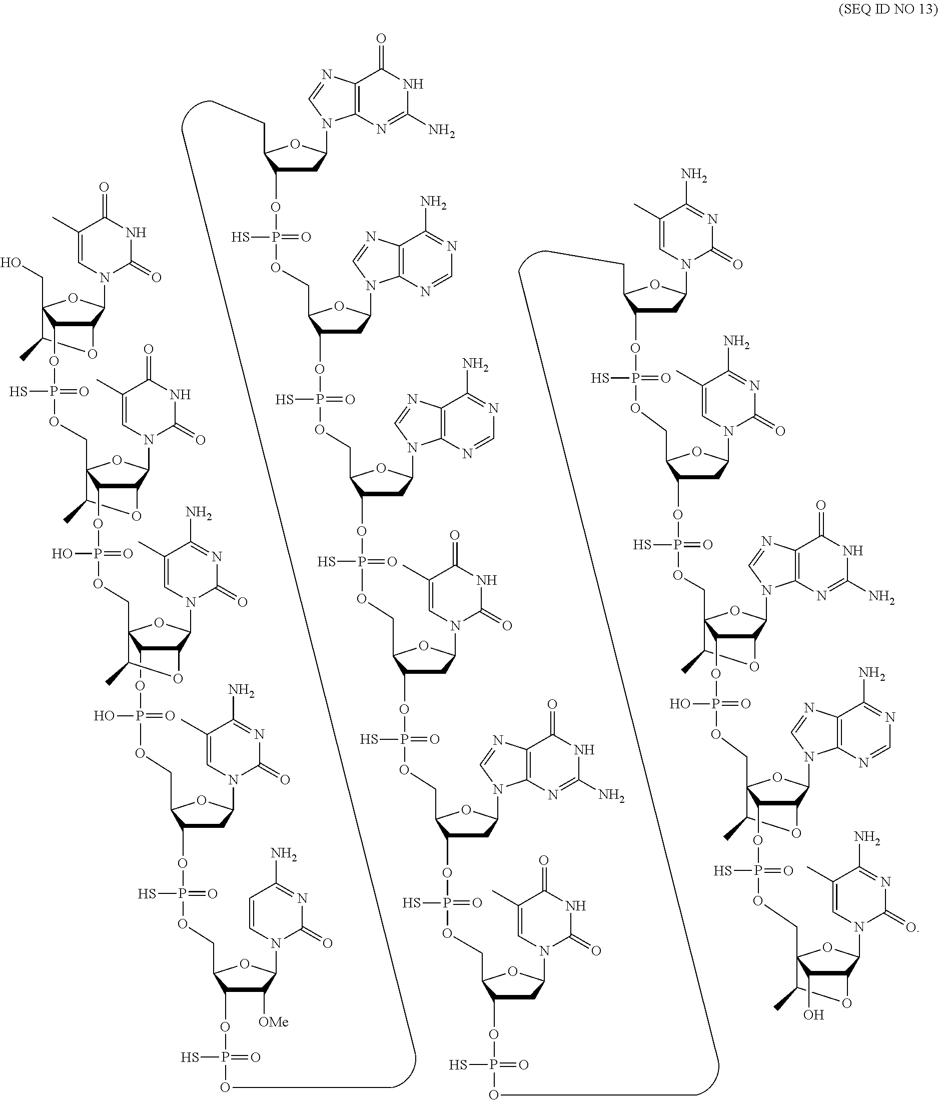

### Structure 1. Compound No. 1522461

In certain embodiments, an oligomeric compound comprises the sodium salt or the potassium salt of the modified oligonucleotide represented by Structure 1.

In certain embodiments, the sodium salt of Compound No. 1522461 is represented by the following chemical structure:

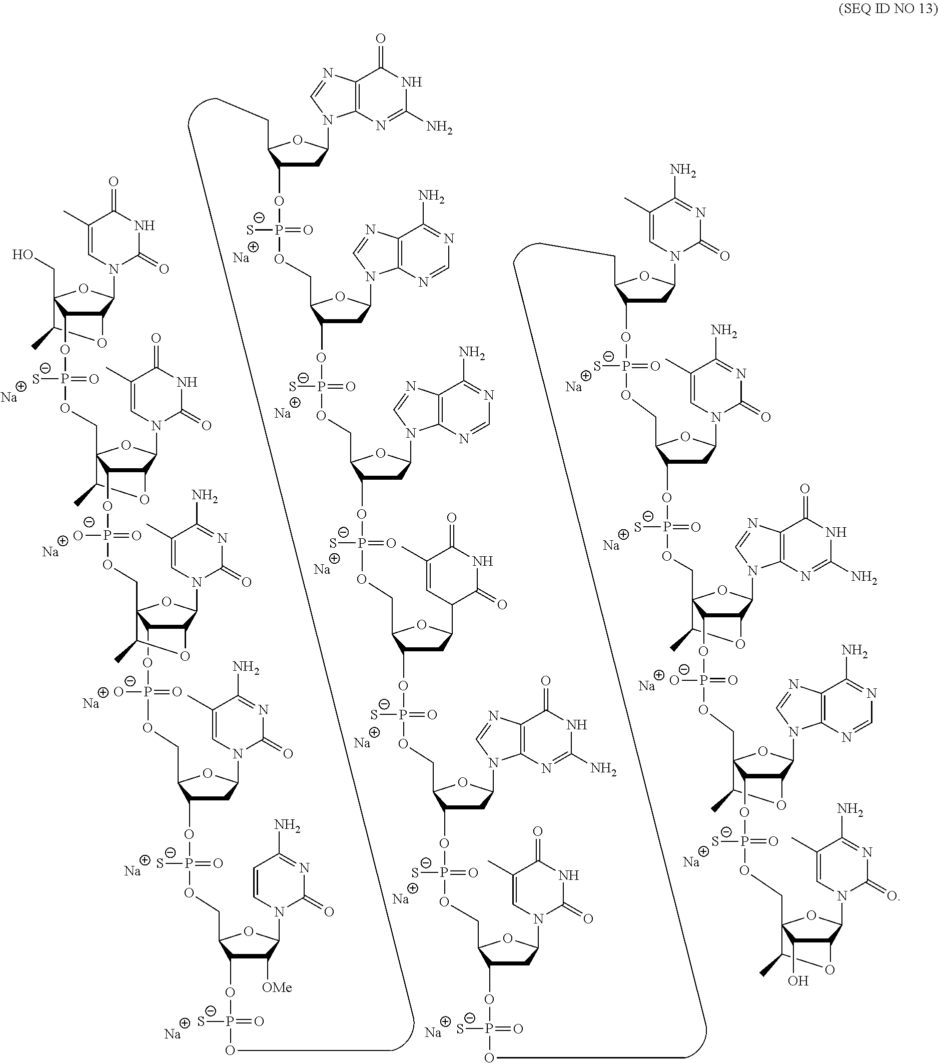

### Structure 2. The Sodium Salt of Compound No. 1522461

In certain embodiments, an oligomeric compound comprises a conjugate group.

In certain embodiments, a prodrug of Compound No. 1522461 is represented by the following chemical structure:

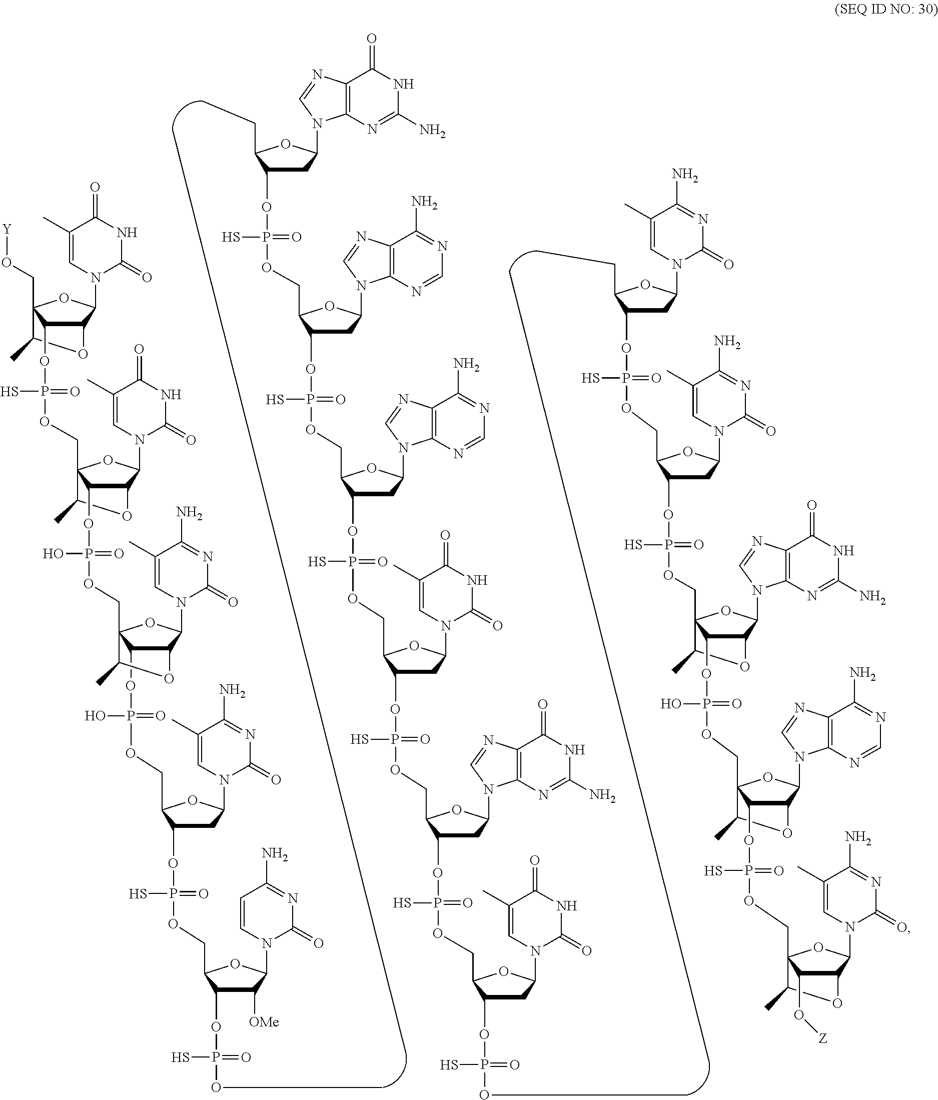

wherein Y and Z are selected from II and a conjugate group, wherein at least one of Y and Z is a conjugate group.

### Structure 3. A Prodrug of Compound No. 1522461

In certain embodiments, a prodrug of Compound No. 1522461 is represented by the following chemical structure:

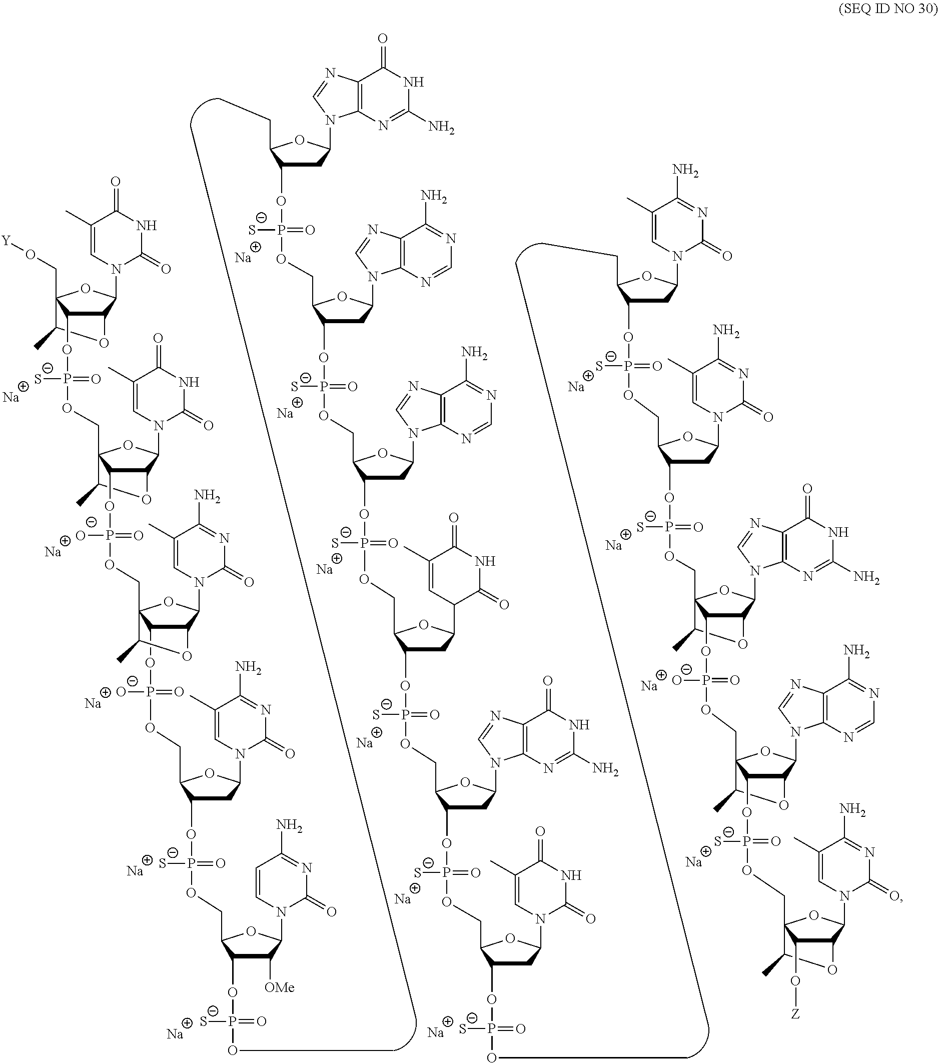

wherein Y and Z are selected from H and a conjugate group, wherein at least one of Y and Z is a conjugate group.

### Structure 4. A Prodrug of Compound No. 1522461

### 2. Compound No. 1400741

In certain embodiments, Compound No. 1400741 is characterized as a mixed wing gapmer of linked nucleosides and having a nucleobase sequence (from 5′ to 3′) of TTCCCGAATGTCCGAC (SEQ ID NO 35), wherein each of nucleosides 1-3 and 14-16 (from 5′ to 3′) are cEt nucleosides, nucleoside 5 is a 2′-OMe nucleoside, and each of nucleosides 4 and 6-13 are 2′-β-D-deoxynucleosides, wherein each internucleoside linkage is a phosphorothioate internucleoside linkage. The cytosines at positions 3-4, 12, 13, and 16 are 5-methylcytosines, while the cytosine at position 5 is a non-methylated cytosine.

In certain embodiments, Compound No. 1400741 is represented by the following chemical notation: TₖₛTₖₛ^{m}Cₖₛ^{m}C_{ds}C_{ys}G_{ds}A_{ds}A_{ds}T_{ds}G_{ds}T_{ds}^{m}C_{ds}^{m}C_{ds}GₖₛAₖₛ^{m}Cₖ (SEQ ID NO: 20), wherein:
A=an adenine nucleobase,
mC=a 5-methylcytosine nucleobase,
C=a cytosine nucleobase,
G=a guanine nucleobase,
T=a thymine nucleobase,
y=a 2′-OMe sugar moiety,
k=a cEt sugar moiety,
d=a 2′-β-D-deoxyribosyl sugar moiety,
s=a phosphorothioate internucleoside linkage, and

In certain embodiments, Compound No. 1400741 is represented by the following chemical structure:

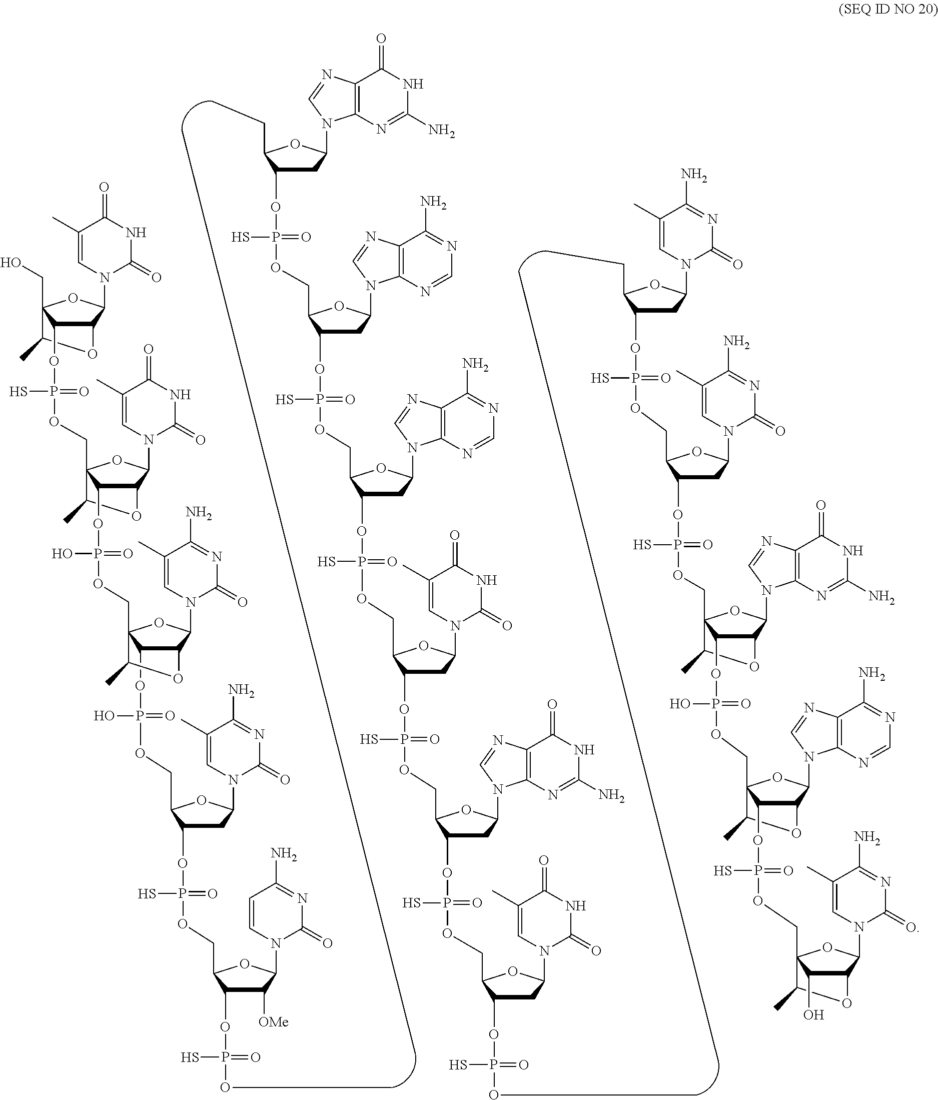

### Structure 5. Compound No. 1400741

In certain embodiments, an oligomeric compound comprises the sodium salt or the potassium salt of the modified oligonucleotide represented by Structure 5.

In certain embodiments, the sodium salt of Compound No. 1400741 is represented by the following chemical

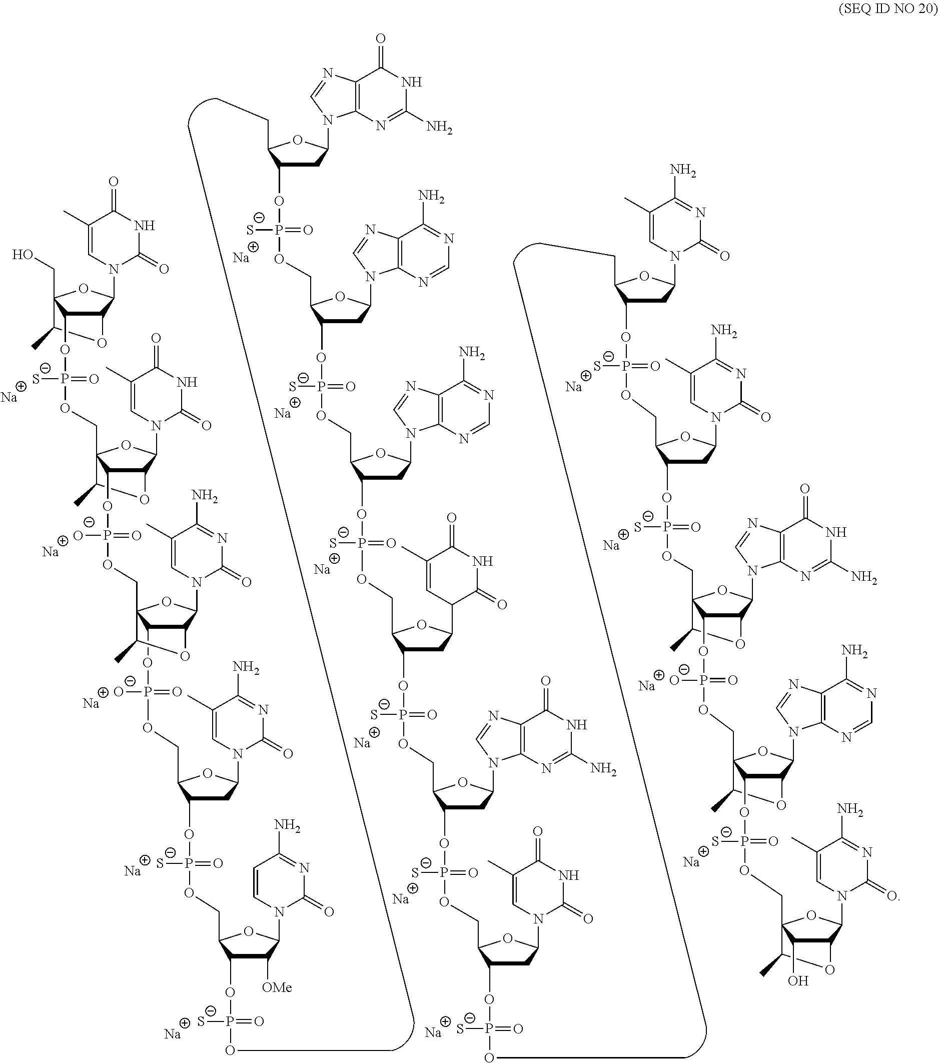

### Structure 6. The Sodium Salt of Compound No. 1400741

In certain embodiments, an oligomeric compound comprises a conjugate group.

In certain embodiments, a prodrug of Compound No. 1400741 is represented by the following chemical structure:

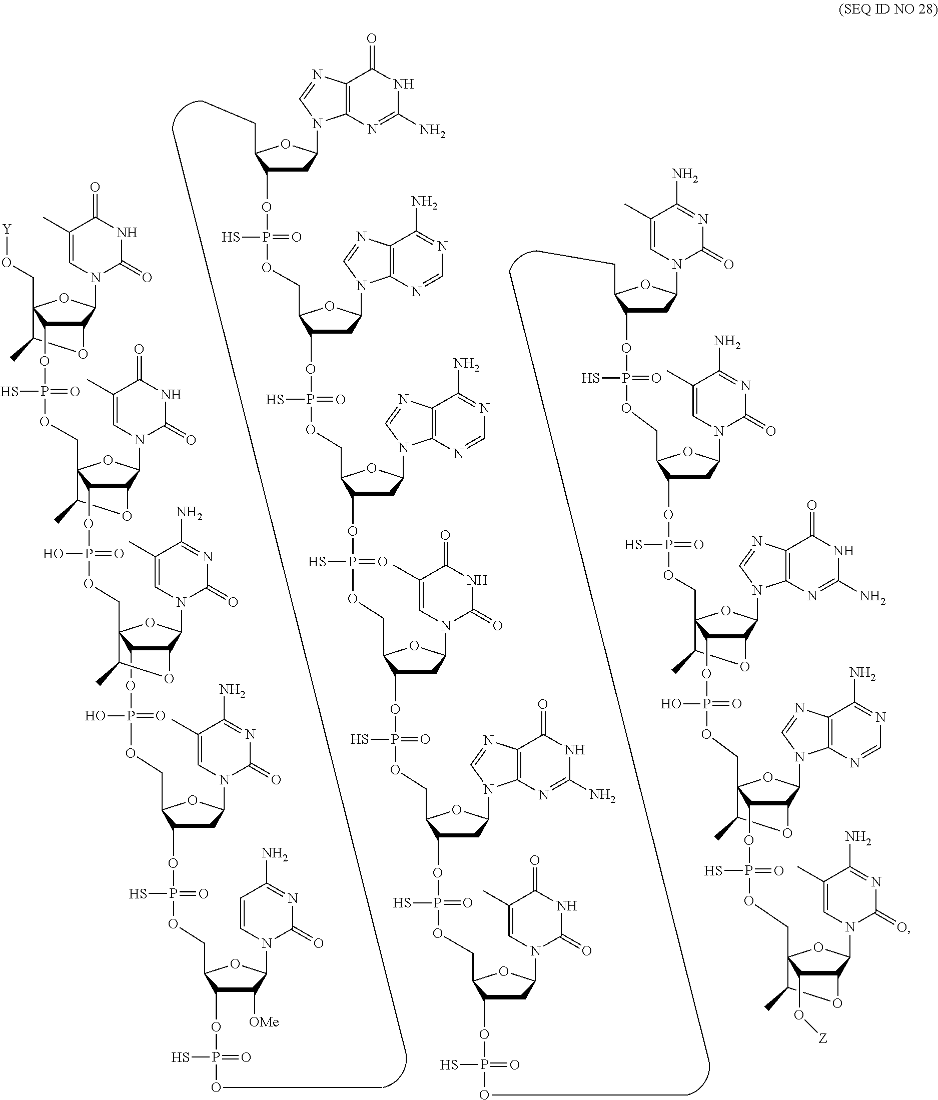

wherein Y and Z are selected from H and a conjugate group, wherein at least one of Y and Z is a conjugate group.

### Structure 7. A Prodrug of Compound No. 1400741

In certain embodiments, a prodrug of Compound No. 1400741 is represented by the following chemical structure:

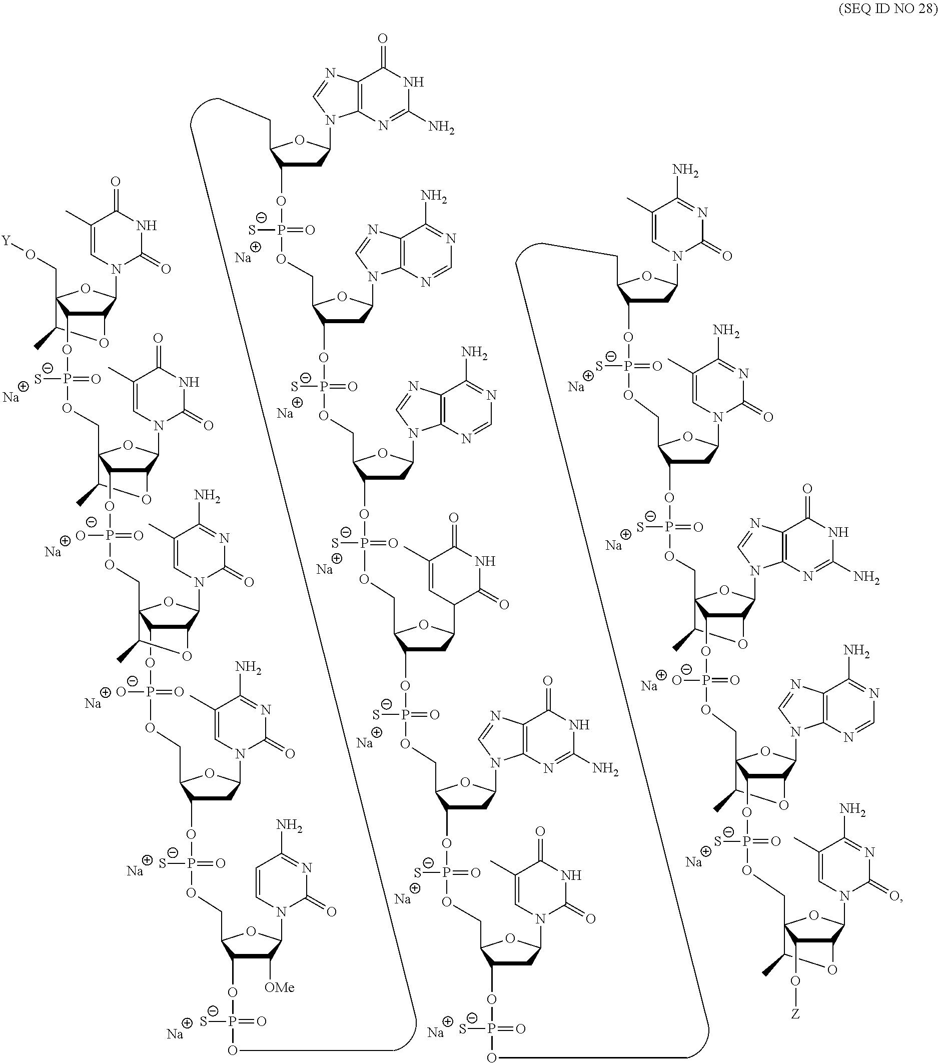

wherein Y and Z are selected from H and a conjugate group, wherein at least one of Y and Z is a conjugate group.

### Structure 8. A Prodrug of Compound No. 1400741

### 3. Compound No. 1522459

In certain embodiments, Compound No. 1522459 is characterized as a mixed wing gapmer of linked nucleosides and having a nucleobase sequence (from 5′ to 3′) of CGAAUGTCCGACAGTG (SEQ ID NO 36), wherein each of nucleosides 1-3 and 14-16 (from 5′ to 3′) are cEt nucleosides, nucleoside 5 is a 2′-OMe nucleoside, and each of nucleosides 4 and 6-13 are 2′-β-D-deoxynucleosides, wherein the internucleoside linkages between nucleosides 2 to 3, 3 to 4 and 14 to 15 are phosphodiester internucleoside linkages, the internucleoside linkages between nucleosides 1 to 2, 4 to 5, 5 to 6, 6 to 7, 7 to 8, 8 to 9, 9 to 10, 10 to 11, 11 to 12, 12 to 13, 13 to 14, and 15 to 16 are phosphorothioate internucleoside linkages. Each cytosine is a 5-methylcytosine.

In certain embodiments, Compound No. 1522459 is represented by the following chemical notation: ^{m}CₖₛGₖₒAₖₒA_{ds}U_{ys}G_{ds}T_{ds}^{m}C_{ds}^{m}C_{ds}G_{ds}A_{ds}^{m}C_{ds}A_{ds}GₖₒTₖₛGₖ (SEQ ID NO: 14), wherein:
A=an adenine nucleobase,
mC=a 5-methylcytosine nucleobase,
U=a uracil nucleobase,
G=a guanine nucleobase,
T=a thymine nucleobase,
y=a 2′-OMe sugar moiety,
k=a cEt sugar moiety,
d=a 2′-β-D-deoxyribosyl sugar moiety,
s=a phosphorothioate internucleoside linkage, and
o=a phosphodiester internucleoside linkage.

In certain embodiments Compound No. 1522459 is represented by the following chemical structure:

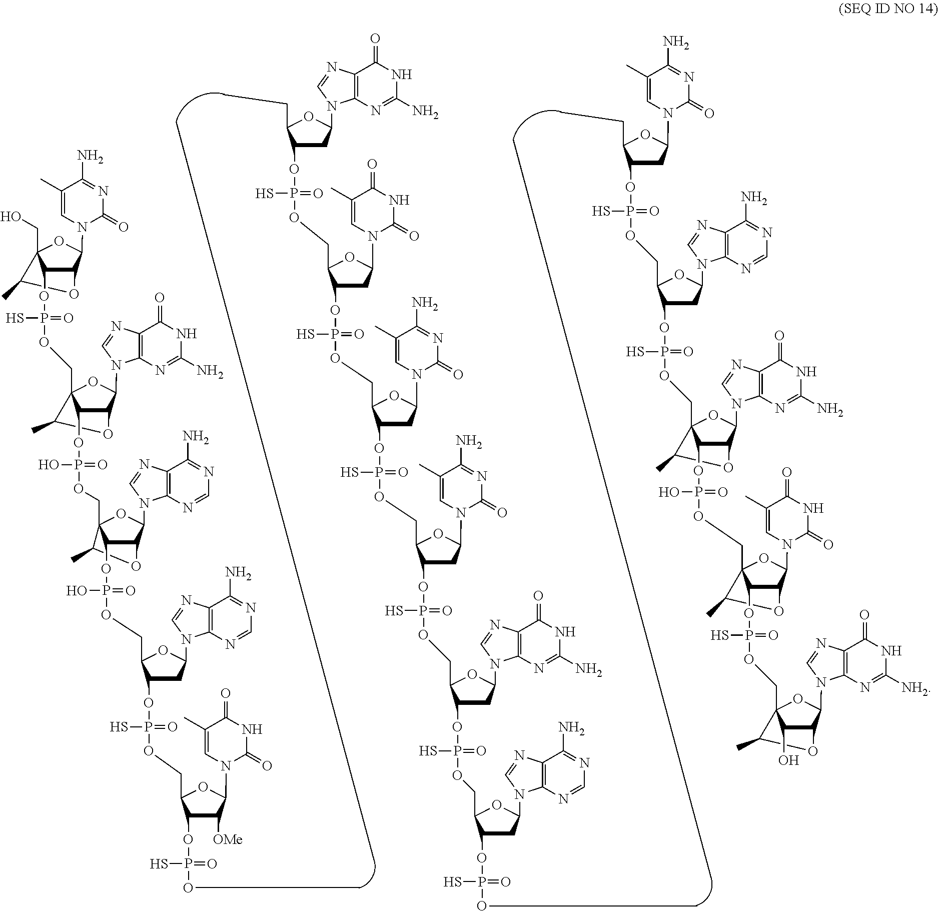

### Structure 9. Compound No. 1522459

In certain embodiments, an oligomeric compound comprises the sodium salt or the potassium salt of the modified oligonucleotide represented by Structure 9.

In certain embodiments the sodium salt of Compound No. 1522459 is represented by the following chemical structure:

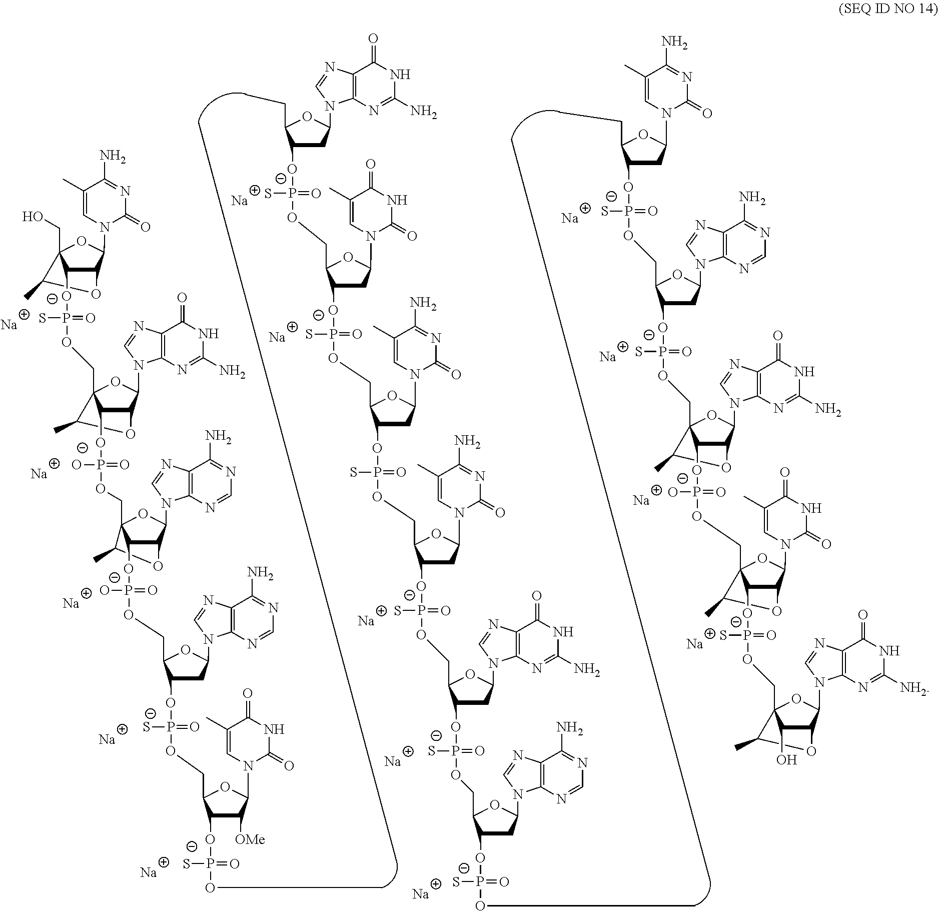

### Structure 10. The sodium salt of Compound No. 1522459

In certain embodiments. an oligomeric compound comprises a conjugate group.

In certain embodiments. a prodrug of Compound No. 1522459 is represented by the following chemical structure:

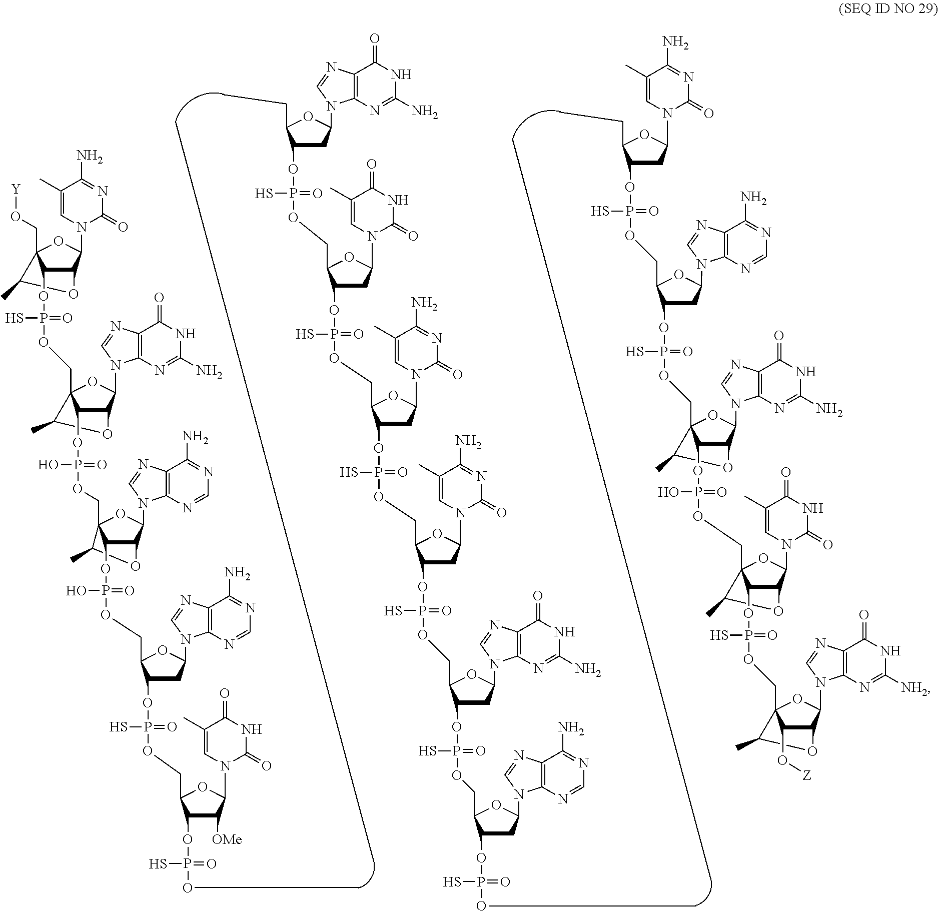

wherein Y and Z are selected from H and a conjugate group, wherein at least one of Y and Z is a conjugate group.

### Structure 11. A prodrug of Compound No. 1522459

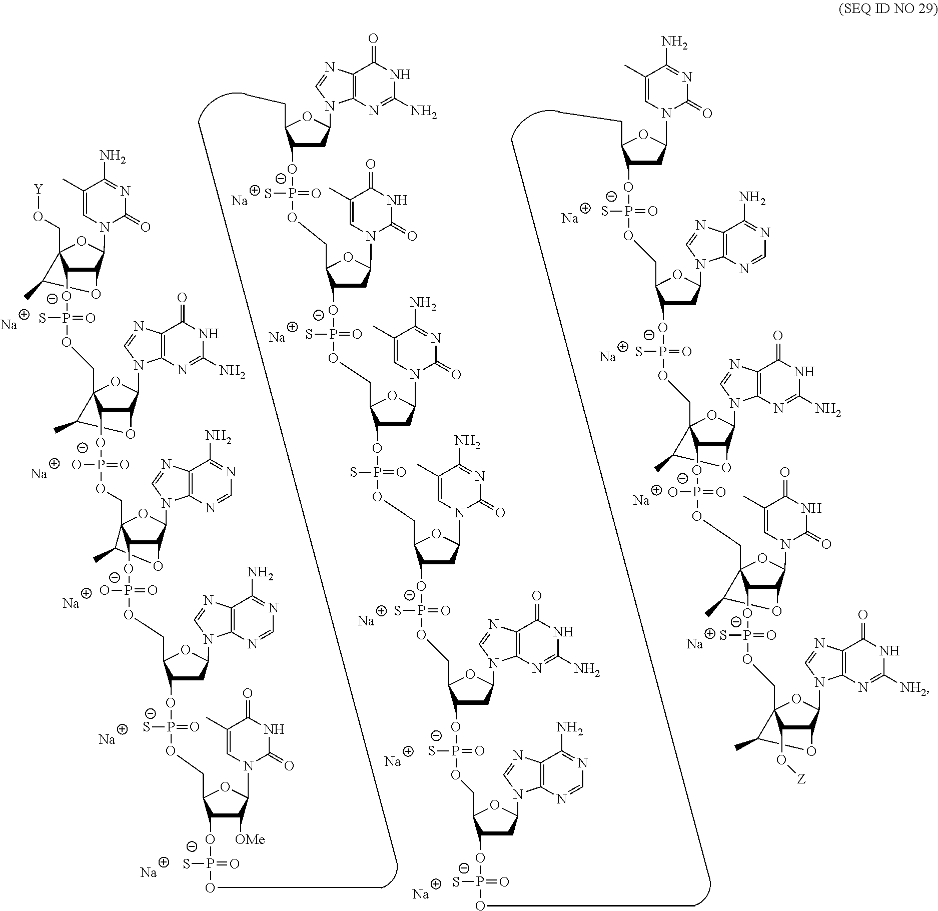

wherein Y and Z are selected from H and a conjugate group, wherein at least one of Y and Z is a conjugate group.

### Structure 12. A prodrug of Compound No. 1522459

### 4. Compound No. 1522464

In certain embodiments, Compound No. 1522464 is characterized as a mixed wing gapmer of linked nucleosides and having a nucleobase sequence (from 5′ to 3′) of CTTTTATTCGCGAGGG (SEQ ID NO 37), wherein each of nucleosides 1-2 and 14-16 (from 5′ to 3′) are cEt nucleosides, nucleoside 3 is a 2′-MOE nucleoside, and each of nucleosides 4-13 are 2′-β-D-deoxynucleosides, wherein the internucleoside linkages between nucleosides 2 to 3, 3 to 4 and 14 to 15 are phosphodiester internucleoside linkages, the internucleoside linkages between nucleosides 1 to 2, 4 to 5, 5 to 6, 6 to 7, 7 to 8, 8 to 9, 9 to 10, 10 to 11, 11 to 12, 12 to 13, 13 to 14, and 15 to 16 are phosphorothioate internucleoside linkages. Each cytosine is a 5-methylcytosine.

In certain embodiments, Compound No. 1522464 is represented by the following chemical notation: ^{m}CₖₛTₖₒTₑₒT_{ds}A_{ds}T_{ds}T_{ds}^{m}C_{ds}G_{ds}^{m}C_{ds}G_{ds}A_{ds}GₖₒGₖₛGₖ (SEQ ID NO: 15), wherein:
A=an adenine nucleobase,
mC=a 5-methylcytosine nucleobase,
G=a guanine nucleobase,
T=a thymine nucleobase,
e=a 2′-OMe sugar moiety,
k=a cEt sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1522464 is represented by the following chemical structure:

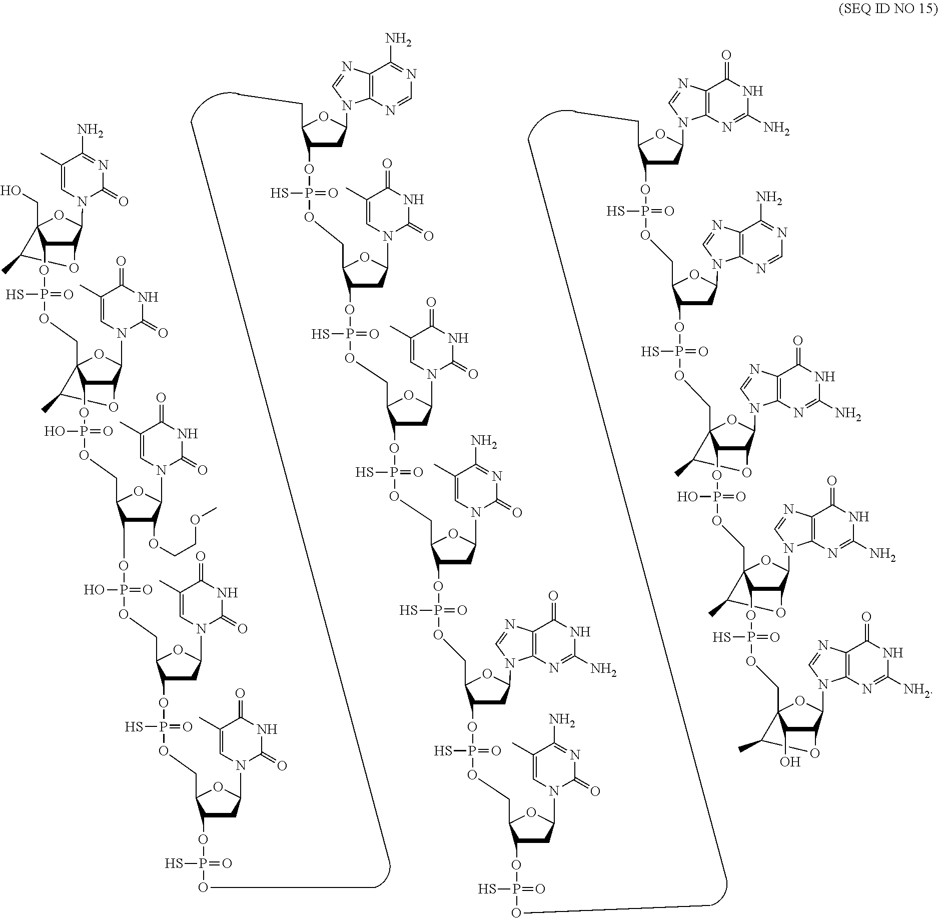

### Structure 13. Compound No. 1522464

In certain embodiments, an oligomeric compound comprises the sodium salt or the potassium salt of the modified oligonucleotide represented by Structure 13.

In certain embodiments, the sodium salt of Compound No. 1522464 is represented by the following chemical

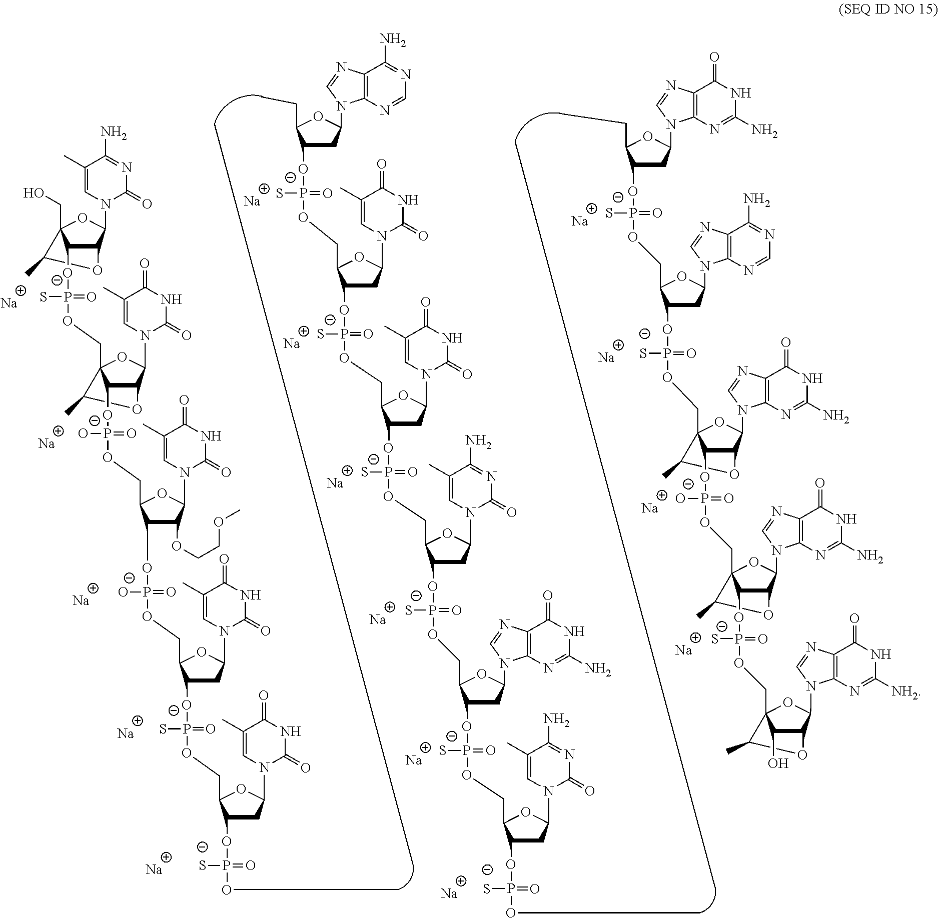

### Structure 14. The sodium salt of Compound No. 1522464

In certain embodiments, an oligomeric compound comprises a conjugate group.

In certain embodiments, a prodrug of Compound No. 1522464 is represented by the following chemical structure:

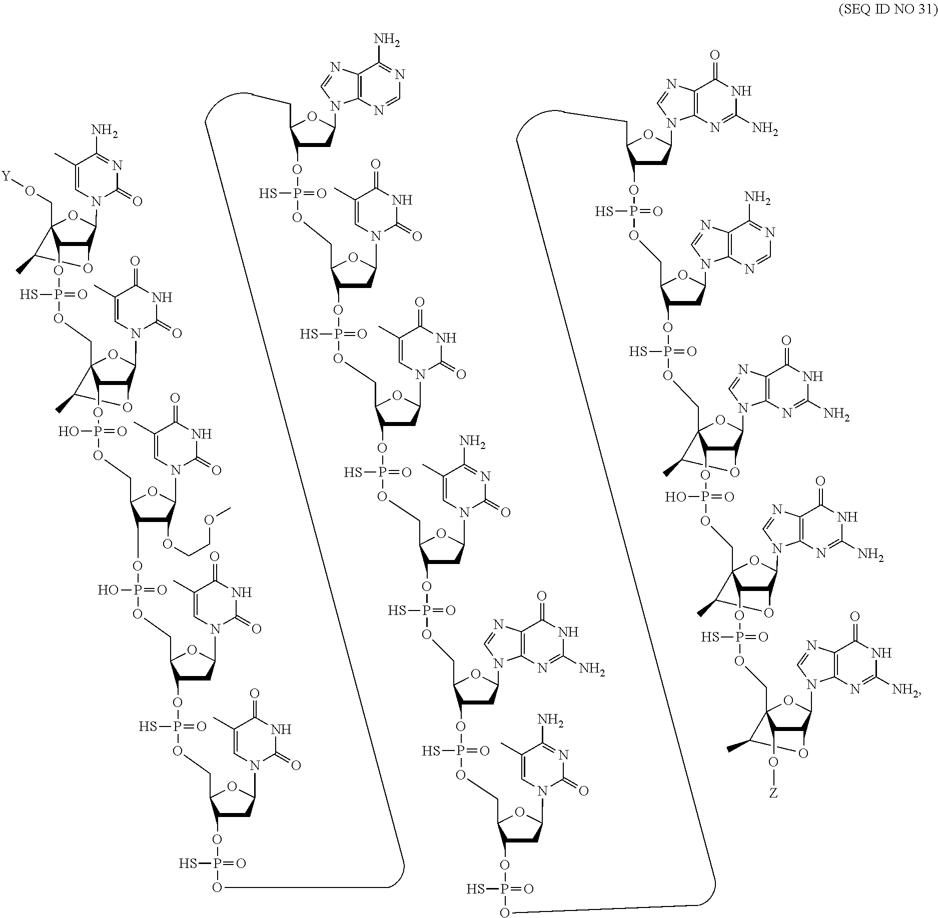

wherein Y and Z are selected from H and a conjugate group, wherein at least one of Y and Z is a conjugate group.

### Structure 15. A prodrug of Compound No. 1522464

In certain embodiments, a prodrug of Compound No. 1522464 is represented by the following chemical structure:

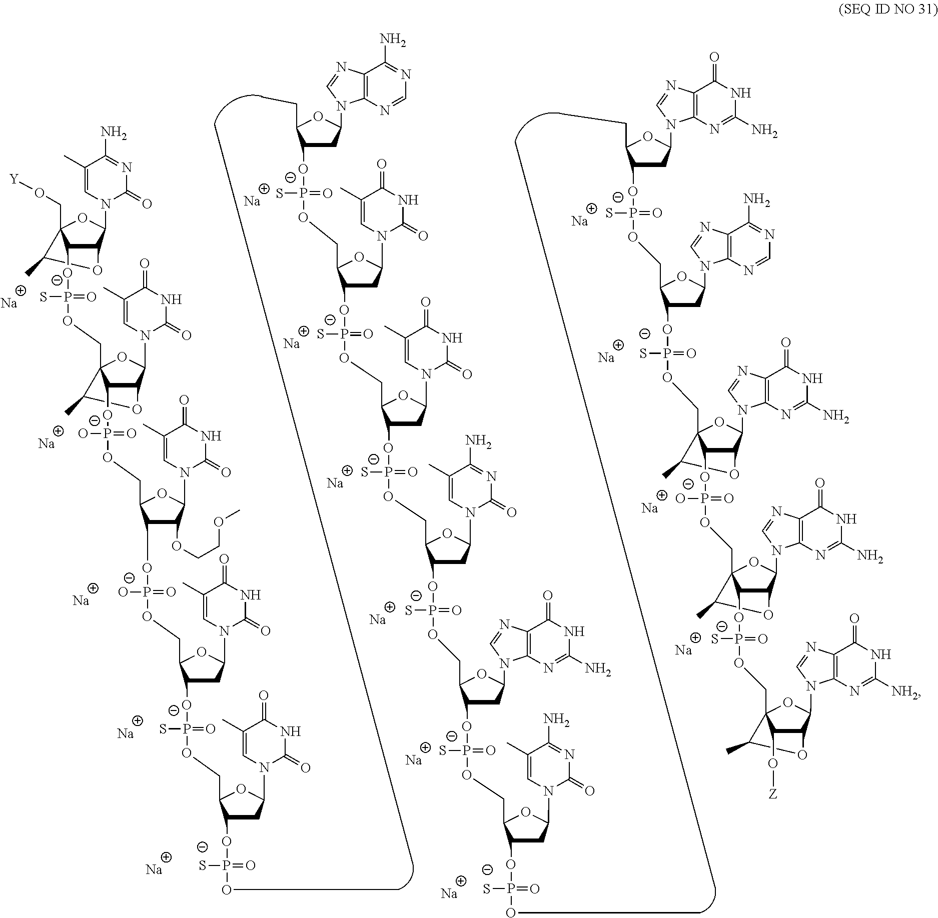

wherein Y and Z are selected from H and a conjugate group, wherein at least one of Y and Z is a conjugate group.

### Structure 16. A prodrug of Compound No. 1522464

### 5. Compound No. 1525073

In certain embodiments, Compound No. 1525073 is characterized as a cEt gapmer of linked nucleosides and having a nucleobase sequence (from 5′ to 3′) of ACAATAAATACCGAGG (SEQ ID NO 33), wherein each of nucleosides 1-3 and 14-16 (from 5′ to 3′) are cEt nucleosides, and each of nucleosides 4-13 are 2′-β-D-deoxynucleosides, wherein the internucleoside linkages between nucleosides 2 to 3, 3 to 4 and 14 to 15 are phosphodiester internucleoside linkages, the internucleoside linkages between nucleosides 1 to 2, 4 to 5, 5 to 6, 6 to 7, 7 to 8, 8 to 9, 9 to 10, 10 to 11, 11 to 12, 12 to 13, 13 to 14, and 15 to 16 are phosphorothioate internucleoside linkages. Each cytosine is a 5-methylcytosine.

In certain embodiments, Compound No. 1525073 is represented by the following chemical notation: Aₖₛ^{m}CₖₒAₖₒA_{ds}T_{ds}A_{ds}A_{ds}T_{ds}A_{ds}^{m}C_{ds}^{m}C_{ds}G_{ds}AₖₒGₖₛGₖ (SEQ ID NO: 11), wherein:
A=an adenine nucleobase,
mC=a 5-methylcytosine nucleobase,
G=a guanine nucleobase,
T=a thymine nucleobase,
k=a cEt sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1525073 is represented by the following chemical structure:

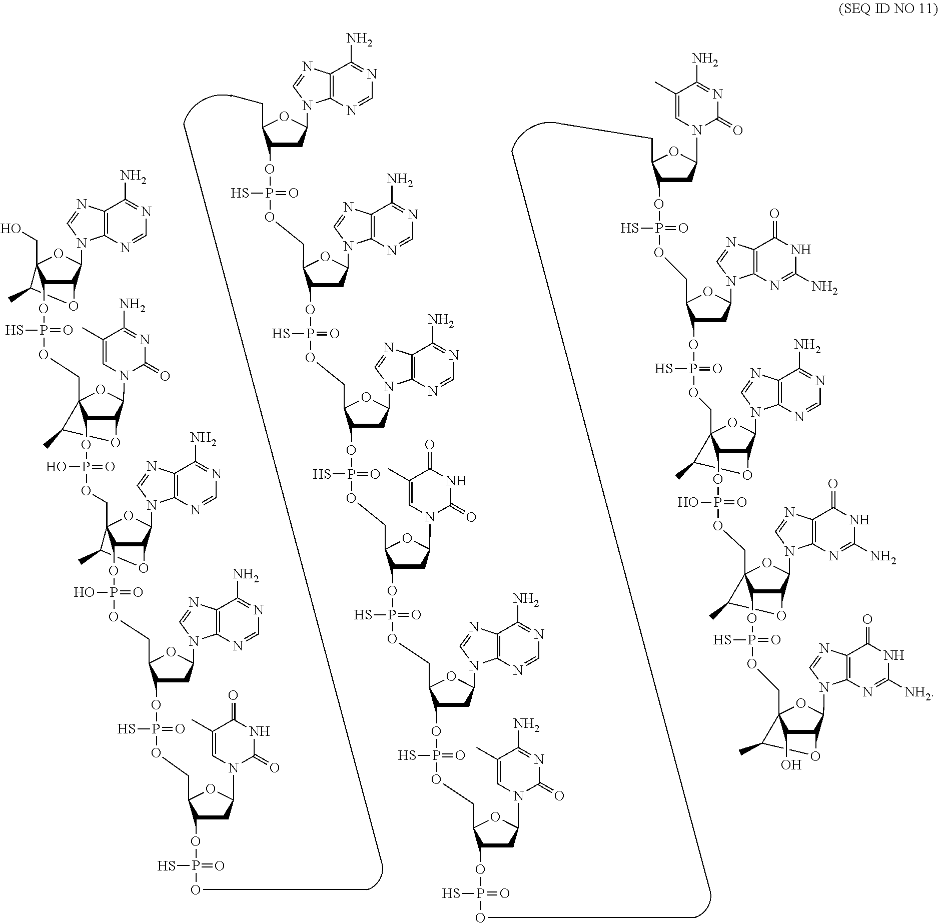

### Structure 17. Compound No. 1525073

In certain embodiments, an oligomeric compound comprises the sodium salt or the potassium salt of the modified oligonucleotide represented by Structure 17.

In certain embodiments, the sodium salt of Compound No. 1525073 is represented by the following chemical structure:

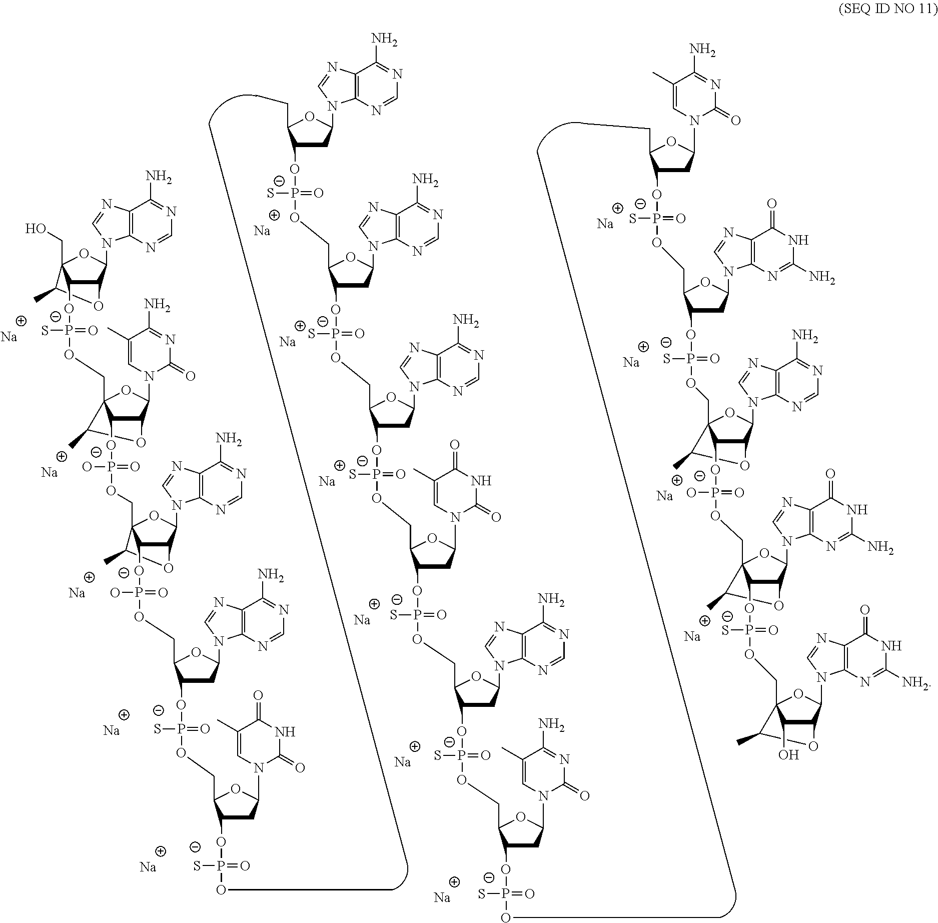

### Structure 18. The Sodium Salt of Compound No. 1525073

In certain embodiments, an oligomeric compound comprises a conjugate group.

In certain embodiments. a prodrug of Compound No. 1525073 is represented by the following chemical structure:

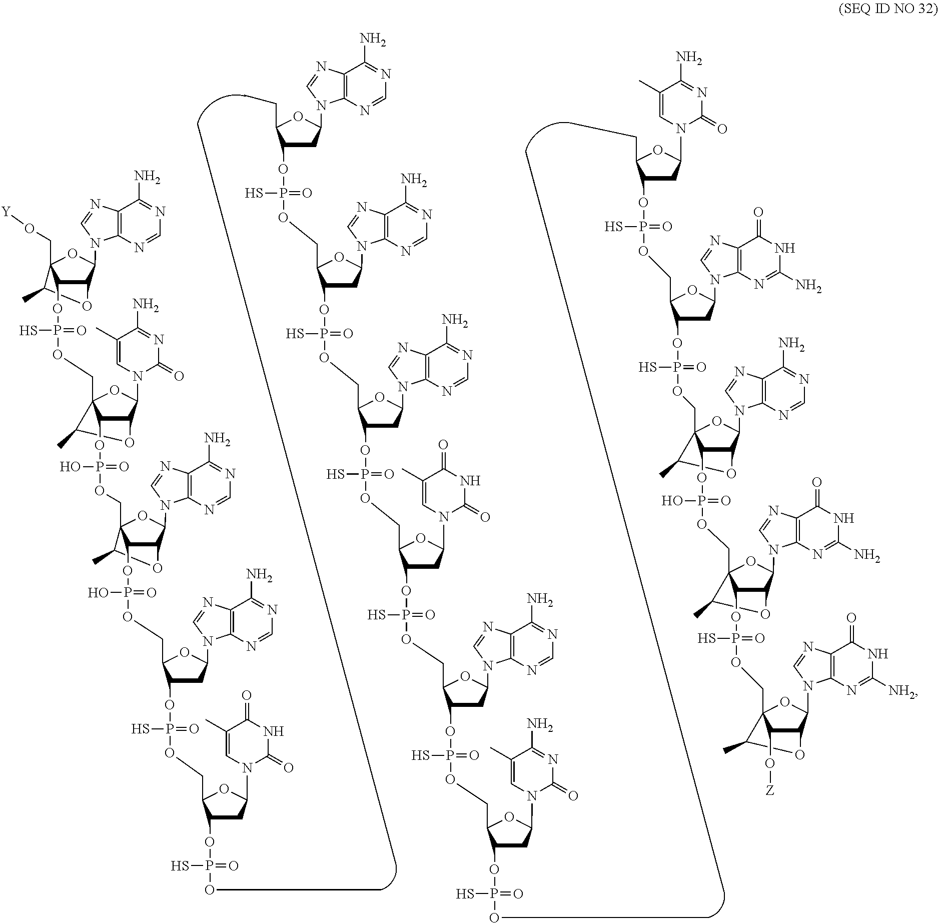

wherein Y and Z are selected from HI and a conjugate group, wherein at least one of Y and Z is a conjugate group.

### Structure 19. A Conjugate of Compound No. 1525073

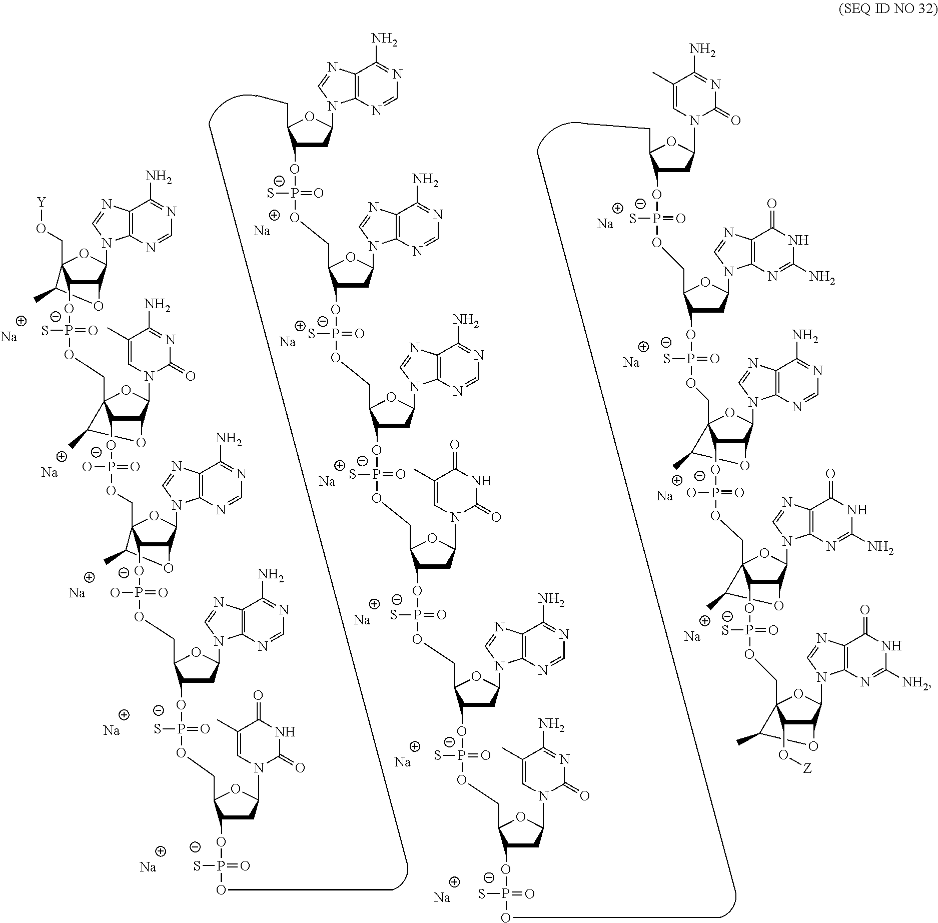

wherein Y and Z are selected from H and a conjugate group, wherein at least one of Y and Z is a conjugate group.

### Structure 20. A Prodrug of Compound No. 1525073

### I. CERTAIN OLIGOUCLEOTIDES

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### A. Certain Modified Nucleosides

Modified nucleosides comprise a modified sugar moiety or a modified nucleobase or both a modifed sugar moiety and a modified nucleobase.

### 1. Certain Sugar Moieties

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 3′4′, and/or 5′ positions. Examples of 2′-substituent groups suitable for non-bicyclic modified sugar moieties include but are not limited to 2′-O(CH₂)₂OCH₃ (“MOE” or “O-methoxyethyl”).

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in e.g., WO 2019/157531. incorporated by reference herein. A 2′-modified sugar moiety has an additional stereocenter at the 2′-position relative to a 2′-deoxyfuranosyl sugar moiety: therefore, such sugar moieties have a total of sixteen possible isomeric configurations. 2′-modified sugar moieties described herein are in the β-D-ribosyl isomeric configuration unless otherwise specified.

### 2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. Examples of modified nucleobases include 5-methylcytosine.

Publications that teach the preparation of certain modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al.; U.S. Pat. No. 6,005,096.

3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages. compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

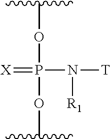

wherein independently for each internucleoside linking group of the modified oligonucleotide:

X is selected from O or S;

R₁ is selected from H, C₁-C₆ alkyl, and substituted C₁-C₆ alkyl; and

T is selected from SO₂R₂, C(═O)R₃, and P(═O)R₄R₅, wherein:

R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;

R₃ is selected from an aryl, a substituted aryl, CH₃, N(CH₃)₂, OCH₃ and a conjugate group;

R₄ is selected from OCH₃, OH, C₁-C₆ alkyl, substituted C₁-C₆ alkyl and a conjugate group; and

R₅ is selected from OCH3, OH, C₁-C₆ alkyl, and substituted C₁-C₆ alkyl.

In certain embodiments, a modified internucleoside linkage comprises a mesyl phosphoramidate linking group having a formula:

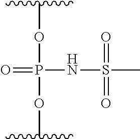 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

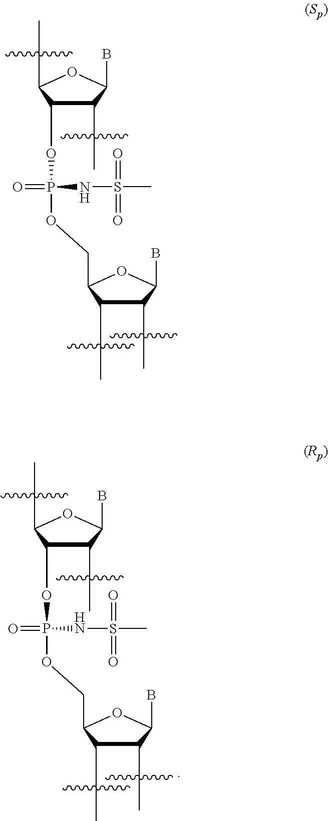

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate or mesyl phosphoramidate linkage. Nonetheless, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art. e.g., methods described in Oka et al., JACS 125, 8307 (2003). Wan et al. Nuc. Acid. Res. 42, 13456 (2014), and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

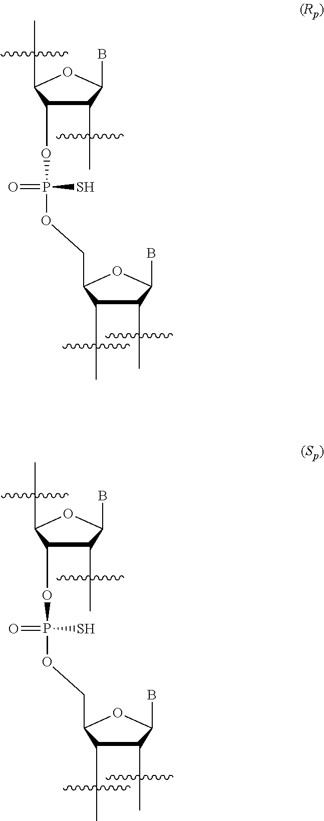

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3-CH₂—N(CH₃)—O-5′), amide-3 (3′—CH₂—C(═O)—N(H)-5′), amide-4 (3′—CH₂—N(H)—C(═O)-5′), formacetal (3′—O—CH₂—O-5′), methoxypropyl (MOP), and thioformacetal (3′—S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

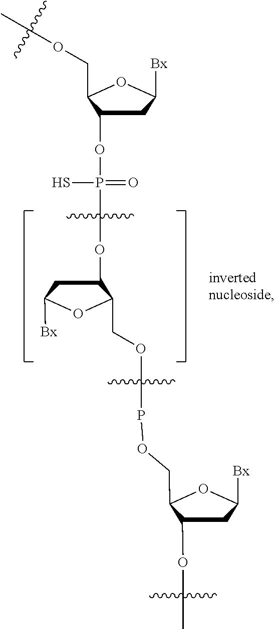

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

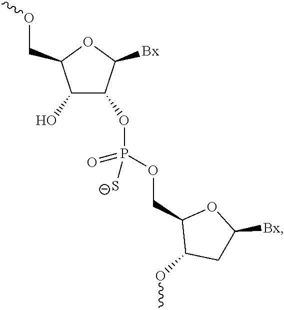

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Gapmer Oligonucleotides

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, one nucleoside of the gap comprises a modified sugar moiety and each remaining nucleoside of the gap comprises a 2′-deoxyribosyl sugar moiety. In certain embodiments, at least one nucleoside of the gap of a gapmer comprises a 2′-OMe sugar moiety.

Herein, the lengths (number of nucleosides) of the three regions of a gapmer may be provided using the notation [ #of nucleosides in the 5′-wing]-[ #of nucleosides in the gap]-[#of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. Thus, a 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing. A 6-10-4 MOE gapmer consists of 6 linked 2′-MOE nucleosides in the 5′-wing, 10 linked 2′-β-D-deoxynucleosides in the gap, and 4 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing.

In certain embodiments, modified oligonucleotides are 5-10-5 MOE gapmers. In certain embodiments, modified oligonucleotides are 6-10-4 MOE gapmers.

In certain embodiments, modified oligonucleotides have a sugar motif selected from 5′ to 3′: eeeeeddddddddddeeeee; wherein each “d” represents a 2′-β-D-deoxyribosyl sugar moiety, and each “e” represents a 2′-MOE sugar moiety.

In certain embodiments, modified oligonucleotides have a sugar motif selected from 5′ to 3′: eeeeeeddddddddddeeee; wherein each “d” represents a 2′-β-D-deoxyribosyl sugar moiety, and each “e” represents a 2′-MOE sugar moiety.

In certain embodiments, modified oligonucleotides are 5-10-5 cEt gapmers.

In certain embodiments, modified oligonucleotides have the sugar motif from 5′ to 3′: kkkddddddddddkkk; wherein each “d” represents a 2′-β-D-deoxyribosyl sugar moiety, and each “k” represents a cEt sugar moiety.

In certain embodiments, modified oligonucleotides have the sugar motif from 5′ to 3′: kkkdyddddddddkkk; wherein each “d” represents a 2′-β-D-deoxyribosyl sugar moiety, each “y” represents a 2′-OMe sugar moiety, and each “k” represents a cEt sugar moiety.

In certain embodiments, modified oligonucleotides have the sugar motif from 5′ to 3′: kkeddddddddddkkk; wherein each “d” represents a 2′-β-D-deoxyribosyl sugar moiety, each “e” represents a 2′-MOE sugar moiety, and each “k” represents a cEt sugar moiety.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, the sugar motif of a modified oligonucleotide is a gapmer and the internucleoside linkages within the gap are all modified. In certain such embodiments, some, or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified oligonucleotides have an internucleoside linkage motif of (5′ to 3′): sooosssssssssssooss wherein each “s” represents a phosphorothioate internucleoside linkage and each “o” represents a phosphodiester internucleoside linkage. In certain embodiments, modified oligonucleotides have an internucleoside linkage motif of (5′ to 3′): sooooossssssssssoss, wherein each “s” represents a phosphorothioate internucleoside linkage and each “o” represents a phosphodiester internucleoside linkage. In certain embodiments, modified oligonucleotides have an internucleoside linkage motif of (5′ to 3′): soossssssssssos, wherein each “s” represents a phosphorothioate internucleoside linkage and each “o” represents a phosphodiester internucleoside linkage.

### CERTAIN OLIGOMERIC COMPOUNDS

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA. 1989, 86, 6553-6556), cholic acid (Manoharan et al., Bioorg. Med. Chem. Lett., 1994. 4. 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992. 20, 533-538), an aliphatic chain, e.g., do-decan-diol or undecyl residues (Saison-Behmoaras et al., EMBO J., 1991. 10. 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1.2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is a lipid having the following structure:

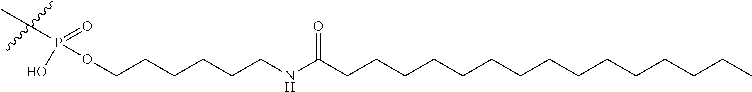 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine. 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to compounds, such as the oligonucleotides provided herein. In general, a bifunctional linking moiety comprises at least two functional groups. One of the functional groups is selected to react with a particular site on a compound and the other is selected to react with a conjugate moiety. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine. 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

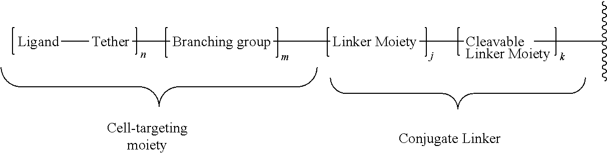

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3. j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

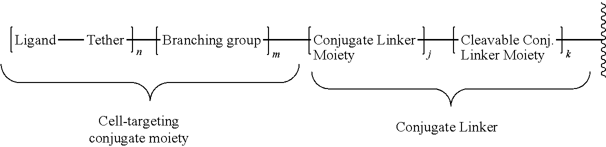

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2. j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety binds a cell surface receptor on a muscle cell. In certain embodiments, the cell-targeting moiety binds a cell surface receptor on a muscle cell. In certain embodiments, the cell-targeting moiety binds a cell surface receptor on a skeletal muscle cell. In some embodiments, the cell-targeting moiety binds a transferrin receptor. In certain embodiments, the cell-targeting moiety is an antibody that binds a transferrin receptor (e.g., Sugo et al., Development of an antibody-siRNA conjugate targeted to cardiac and skeletal muscles. J Controlled Release 237:1-13 (2016)). In some embodiments, the antibody that binds a transferrin receptor is a humanized antibody, a chimeric antibody, a monoclonal antibody, or a recombinant or engineered version thereof. In certain embodiments, the cell-targeting moiety is an antibody fragment that binds a transferrin receptor. In some embodiments, the antibody fragment that binds a transferrin receptor is a F(ab′)₂, a Fab, a Fab′, a Fv, recombinant or engineered versions thereof, or engineered peptides.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, terminal groups comprise one or more 2′-linked nucleosides or sugar moieties. In certain such embodiments, the 2′-linked group is an abasic sugar moiety.

### II. ANTISENSE ACTIVITY

In certain embodiments, oligomeric compounds and oligomeric duplexes are capable of hybridizing to a target nucleic acid, resulting in at least one antisense activity: such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments. antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments. described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA or dsRNAi) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### III. CERTAIN TARGET NUCLEIC ACIDS

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

### A. DMPK

In certain embodiments, oligomeric compounds comprise or consist of an oligonucleotide comprising a region that is complementary to a target nucleic acid, wherein the target nucleic acid is a DMPK nucleic acid. In certain embodiments, a DMPK nucleic acid has the sequence set forth in SEQ ID NO: 1 (the complement of GENBANK Accession No. NT_011109.16, truncated from nucleotides 18539000 to 18566000). SEQ ID NO: 2 (GENBANK Accession No. NM_004409.4). In certain embodiments, a DMPK nucleic acid has the sequence set forth in SEQ ID NO: 3 (the complement of GENBANK Accession No. NC_000019.10, truncated from nucleotides 45767001 to 45786000). SEQ ID NO: 4 (GENBANK Accession No. NM_001288764.1), and/or SEQ ID NO: 5 (GENBANK Accession No. NM_001081560.2).

In certain embodiments, contacting a cell with an oligomeric compound complementary to SEQ ID NO: 1 or SEQ ID NO: 2 reduces the amount of DMPK RNA, and in certain embodiments reduces the amount of DMPK protein. In certain embodiments, contacting a cell with an oligomeric compound complementary to SEQ ID NO: 3. SEQ ID NO: 4, and/or SEQ ID NO: 5 reduces the amount of DMPK RNA, and in certain embodiments reduces the amount of DMPK protein. In certain embodiments, the oligomeric compound consists of a modified oligonucleotide. In certain embodiments, the oligomeric compound consists of a modified oligonucleotide and a conjugate group.

### B. Certain Target Nucleic Acids In Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are muscle tissues, such as tibialis anterior, gastrocnemius, and quadriceps muscles. In certain embodiments, the pharmacologically relevant tissue is heart muscle tissue. In certain embodiments, the target nucleic acid is expressed in a pharmacologically relevant cell. In certain embodiments the pharmacologically relevant cell is a muscle cell. In certain embodiments the pharmacologically relevant cell is a skeletal muscle cell.

### IV. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade artificial cerebrospinal fluid. In certain embodiments, a pharmaceutical composition comprises a modified oligonucleotide and PBS. In certain embodiments, a pharmaceutical composition consists of a modified oligonucleotide and PBS. In certain embodiments, a pharmaceutical composition consists essentially of a modified oligonucleotide and PBS. In certain embodiments, the PBS is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono-or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution. Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers, Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na⁺ ions. However, the mass of the protons is nevertheless counted toward the weight of the dose, and the mass of the Na⁺ ions is not counted toward the weight of the dose. Thus, for example, a dose, or dosage unit, of 10 mg of Compound No. 1522461. equals the number of fully protonated molecules that weighs 10 mg. This would be equivalent to 10.62 mg of solvent-free, sodium acetate-free, anhydrous sodiated Compound No. 1522461. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### Certain Comparator Compositions

In certain embodiments. ISIS-DMPKR_{Rx} (generic name baliforsen: Compound No. 598769), entered into clinical trials for treatment of DM1. is a comparator compound (see, e.g., Thorton, et al., Neurology. 86(16 supplement): P3.163, 2016). ISIS-DMPK_{Rx}, 598769 was previously described in WO2015/021457. incorporated herein by reference, and has a nucleobase sequence (from 5′ to 3′) of TCCCGAATGTCCGACA (SEQ ID NO: 34). The sugar motif for Compound No. 598769 is (from 5′ to 3′): eekkddddddddkkee; wherein each “e” represents a 2′-MOE sugar moiety, each “d” represents a 2′-β-D-deoxyribosyl sugar moiety, and each “k” represents a cEt sugar moiety. The internucleoside linkage motif for Compound No. 598769 is (from 5′ to 3′): sssssssssssssss; wherein each “s” represents a phosphorothioate internucleoside linkage. Each cytosine nucleobase in Compound No. 598769 is a 5-methylcytosine.

In certain embodiments, Compound No. 486178. although not entered into clinical trials, is a comparator compound (see, e.g., Yadava, et al., Hum. Mol. Genetics. 29(9): 1440-1453, 2020; Pandey, et al., J. Pharamacol. Expt. Therapy, 355(2):329-340. 2015). Compound No. 486178 was previously described in WO 2015/021457 A2. WO 2017/053995 A1, and WO 2019/118916 A1. each of which is incorporated herein by reference, and consists of the nucleobase sequence (from 5′ to 3′): ACAATAAATACCGAGG (SEQ ID NO: 33). The sugar motif for Compound No. 486178 is (from 5′ to 3′): kkkddddddddddkkk: wherein each “d” represents a 2′-β-D-deoxyribosyl sugar moiety, and each “k” represents a cEt sugar moiety. The internucleoside linkage motif for Compound No. 486178 is (from 5′ to 3′): sssssssssssssss; wherein each “s” represents a phosphorothioate internucleoside linkage. Each cytosine nucleobase in Compound No. 486178 is a 5-methylcytosine.

In certain embodiments, compounds described herein are superior relative to compounds described WO2015/021457, because they demonstrate one or more improved properties, such as activity, potency, and/or tolerability.

For example, Compound No. 1400741, Compound No. 1522459, and Compound No. 1522461, Compound No. 1522464, and Compound No. 1525073 each demonstrated improved potency in vitro as compared to Compound No. 486178.

As shown in Example 2, Compound No. 1400741, Compound No. 1522459 and Compound No. 1522461, Compound No. 1522464, and Compound No. 1525073 achieved an in vitro IC₅₀ of 0.04 μM, 0.03 μM, 0.09 μM, 0.21 μM, and 0.18 μM, respectively. In comparison, Compound No. 486178 achieved an in vitro IC₅₀ of >2 μM. Therefore. each of Compound No. 1400741, Compound No. 1522459, and Compound No. 1522461, Compound No. 1522464, and Compound No. 1525073 are more potent than Compound No. 486178 in this assay.

For example, Compound No. 1400741, Compound No. 1522459, and Compound No. 1522461, Compound No. 1522464, and Compound No. 1525073 each demonstrated reduced liver toxicity compared to compound No. 598769. In order to assess liver toxicity in a short time frame, each compound was conjugated to a triantennary THA-GalNAc moiety, shown below. This ensures delivery of the modified oligonucleotide to the liver, and shows liver toxicity that otherwise might not be detected outside of longer term, repeat-dosing studies.

For example, at 96 hours after dosing, as shown in the table below. Compound No. 1400741 conjugated to THA-GalNAc, Compound. No. 1525079, achieves an ALT value of 54 (U/L), and an AST value of 62 (U/L). Compound No. 1522459 conjugated to THA-GalNAc, Compound. No. 1522487, achieves an ALT value of 30 (U/L), and an AST value of 49 (U/L). Compound No. 1522461conjugated to THA-GalNAc. Compound. No. 1522489, achieves an ALT value of 46 (U/L), and an AST value of 76 (U/L). Compound No. 1522464 conjugated to THA-GalNAc, Compound. No. 1522492, achieves an ALT value of 65 (U/L), and an AST value of 81 (U/L). Compound No. 1525073 conjugated to THA-GalNAc, Compound. No. 1525089, achieves an ALT value of 67(U/L), and an AST value of 70 (U/L). In comparison, Compound No. 1525074, Compound No. 598769 conjugated to THA-GalNACc, achieved an ALT value of 848 (U/L) and an AST value of 600 (U/L). Therefore, each of Compound No. 1400741, Compound No. 1522459, and Compound No. 1522461, Compound No. 1522464, and Compound No. 1525073 are more tolerable than Compound No. 598769 in this assay.

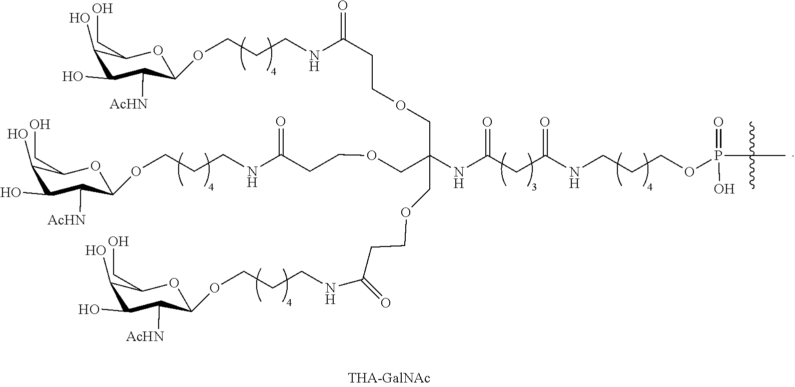

TABLE 1
Plasma chemistry markers in BALB/c mice for
GalNAc-conjugated oligomeric compounds

| | Unconjugated | | |
|---|---|---|---|
| | Parent | | |
| Compound | Compound | | |
| No. | No. | ALT (U/L) | AST(U/L) |
| PBS | N/A | 61 | 149 |
| 1525074 | 598769 | 848 | 600 |
| 1525079 | 1400741 | 54 | 62 |
| 1522487 | 1522459 | 30 | 49 |
| 1522489 | 1522461 | 46 | 76 |
| 1522492 | 1522464 | 65 | 81 |
| 1525089 | 1525073 | 67 | 70 |

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, ENSEMBL identifiers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG.” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of 160, and ³³S, 34S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Design of Modified Oligonucleotides Complementary to Human DMPK Nucleic Acid

Modified oligonucleotides complementary to a human DMPK nucleic acid were designed, as described in the tables below. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (the complement of GENBANK Accession No. NT_011109.16, truncated from nucleotides 18539000 to 18566000), or to SEQ ID NO: 2 (GENBANK Accession No. NM_004409.4), or to both. Compound 598769 (ISIS DMPK-Rx) and Compound No. 486178 were previously described in WO2015/021457.

| | | Start | Stop | Start | Start | |
|---|---|---|---|---|---|---|
| | | Site | Site | Site | Site | |
| | | SEQ | SEQ | SEQ | SEQ | |
| | Chemistry | ID | ID | ID | ID | SEQ |
| Compound | Notation | NO: | NO: | NO: | NO: | ID |
| Number | (5′-3′) | 1 | 1 | 2 | 2 | NO. |
| 486178 | Aₖₛ^{m}CₖₛAₖₛA_{ds} | 24730 | 24745 | 2788 | 2803 | 19 |
| | T_{ds}A_{ds}A_{ds}A_{ds} | | | | | |
| | T_{ds}A_{ds}^{m}C_{ds}^{m}C_{ds} | | | | | |
| | G_{ds}AₖₛGₖₛGₖ | | | | | |
| 598769 | Tₑₛ^{m}Cₑₛ^{m}Cₖₛ | 19498 | 19513 | 1359 | 1374 | 12 |
| | ^{m}CₖₛG_{ds}A_{ds}A_{ds} | | | | | |
| | T_{ds}G_{ds}T_{ds}^{m}C_{ds} | | | | | |
| | ^{m}C_{ds}GₖₛAₖₛ | | | | | |
| | ^{m}CₑₛAₑ | | | | | |
| 1400741 | TₖₛTₖₛ^{m}Cₖₛ | 19499 | 19514 | 1360 | 1375 | 20 |
| | ^{m}C_{ds}C_{ys}G_{ds} | | | | | |
| | A_{ds}A_{ds}T_{ds}G_{ds} | | | | | |
| | Tₛ^{m}C_{ds}^{m}C_{ds}Gₖₛ | | | | | |
| | Aₖₛ^{m}Cₖ | | | | | |
| 1522459 | ^{m}CₖₛGₖₒAₖₒA_{ds} | 19495 | 19510 | 1356 | 1371 | 14 |
| | U_{ys}G_{ds}T_{ds}^{m}C_{ds} | | | | | |
| | ^{m}C_{ds}G_{ds}A_{ds}^{m}C_{ds} | | | | | |
| | A_{ds}GₖₒTₖₛGₖ | | | | | |
| 1522461 | TₖₛTₖₒ^{m}Cₖₒ | 19499 | 19514 | 1360 | 1375 | 13 |
| | ^{m}C_{ds}C_{ys}G_{ds}A_{ds} | | | | | |
| | A_{ds}T_{ds}G_{ds}T_{ds} | | | | | |
| | ^{m}C_{ds}^{m}C_{ds}GₖₒAₖₛ | | | | | |
| | ^{m}Cₖ | | | | | |
| 1522464 | ^{m}CₖₛTₖₒTₑₒT_{ds} | 24775 | 24790 | 2833 | 2848 | 15 |
| | T_{ds}A_{ds}T_{ds}T_{ds} | | | | | |
| | ^{m}C_{ds}G_{ds}^{m}C_{ds} | | | | | |
| | G_{ds}A_{ds}GₖₒGₖₛ | | | | | |
| | Gₖ | | | | | |
| 1525073 | Aₖₛ^{m}CₖₒAₖₒA_{ds} | 24730 | 24745 | 2788 | 2803 | 11 |
| | T_{ds}A_{ds}A_{ds}A_{ds} | | | | | |
| | T_{ds}A_{ds}^{m}C_{ds}^{m}C_{ds} | | | | | |
| | G_{ds}AₖₒGₖₛGₖ | | | | | |

A subscript “k” represents a cEt sugar moiety, a subscript “y” represents a 2′-OMe sugar moiety, a subscript “e” represents a 2′-MOE sugar moiety, a subscript “d” represents a 2′-β-D-deoxyribosyl sugar moiety, a subscript “s” indicates a phosphorothioate internucleoside linkage, a subscript “o” represents a phosphodiester internucleoside linkage, and superscript “m” before a C represents a 5-methylcytosine, while a bold, underlined, italicized “C” without a superscript “m” is a non-methylated cytosine.

### Example 2: Dose-Dependent Inhibition of Human DMPK in A431 Cells by Modified Oligonucleotides

Modified oligonucleotides were tested at various doses in A431 cells. Cells were plated at a density of 11,000 cells per well and were treated using free uptake with modified oligonucleotides at various doses, as specified in the table below. After a treatment period of approximately 48 hours, DMPK RNA levels were measured by quantitative real-time RTPCR using the human DMPK primer-probe set RTS38095 (forward sequence CTGAGCCGGGAGATGGA, designated herein as SEQ ID NO: 6; reverse sequence GGACGTGTGCCTCTAGGT, designated herein as SEQ ID NO: 7; probe sequence TGACTGGCGAAGTTCTGGTTGTCC, designated herein as SEQ ID NO: 8). DMPK RNA levels were normalized to total RNA, as measured by human GAPDH. Human GAPDH was amplified using the human primer probe set RTS104 (forward sequence GAAGGTGAAGGTCGGAGTC, designated herein as SEQ ID NO: 16; reverse sequence GAAGATGGTGATGGGATTTC, designated herein as SEQ ID NO: 17; probe sequence CAAGCTTCCCGTTCTCAGCC, designated herein as SEQ ID NO: 18). Results are presented as percent DMPK RNA, relative to the amount in untreated control cells (% UTC).

The half maximal inhibitory concentration (ICso) of each modified oligonucleotide was calculated using the log (inhibitor) vs. normalized response-variable slope in GraphPad Prism and is also presented in the tables below.

TABLE 2
Dose-dependent reduction of human DMPK RNA
in A431 cells by modified oligonucleotides

| Compound | DMPK RNA (% UTC) | | | | IC₅₀ |
|---|---|---|---|---|---|
| No. | 31 nM | 125 nM | 500 nM | 2000 nM | (μM) |
| 486178 | 108 | 83 | 78 | 54 | >2.0 |

TABLE 3
Dose-dependent reduction of human DMPK RNA in A431 cells by modified oligonucleotides

| Compound | DMPK RNA (% UTC) | | | | | | | | | IC₅₀ |
|---|---|---|---|---|---|---|---|---|---|---|
| No. | 3 nM | 9 nM | 27 nM | 82 nM | 247 nM | 741 nM | 2222 nM | 6667 nM | 20000 nM | (μM) |
| 598769 | 88 | 85 | 58 | 38 | 21 | 8 | 5 | 5 | 2 | 0.05 |
| 1400741 | 90 | 75 | 62 | 36 | 17 | 14 | 9 | 5 | 5 | 0.04 |
| 1522459 | 87 | 80 | 55 | 31 | 9 | 5 | 3 | 2 | 2 | 0.03 |
| 1522461 | 84 | 76 | 75 | 53 | 31 | 17 | 15 | 9 | 8 | 0.09 |
| 1522464 | 78 | 78 | 76 | 71 | 50 | 30 | 21 | 15 | 10 | 0.21 |
| 1525073 | 85 | 89 | 80 | 64 | 44 | 28 | 14 | 7 | 4 | 0.18 |

### Example 3: Dose-Dependent Inhibition of Human DMPK in SH-SY5Y Cells by Modified Oligonucleotides

Modified oligonucleotides were tested at various doses in SH-SY5Y cells. Cells were plated at a density of 35,000 cells per well and were treated using electroporation with modified oligonucleotides at various doses, as specified in the table below. After a treatment period of approximately 24 hours, DMPK RNA levels were measured by quantitative real-time RTPCR using the human DMPK primer-probe set RTS38095 (described herein above). DMPK RNA levels were normalized to total RNA, as measured by human GAPDH. Human GAPDH was amplified using the human primer probe set RTS104 (described herein above). Results are presented as percent of DMPK RNA, relative to the amount in untreated control cells (% UTC).

The half maximal inhibitory concentration (IC₅₀) of each modified oligonucleotide was calculated using the log (inhibitor) vs. normalized response—variable slope in GraphPad Prism and is also presented in the tables below.

TABLE 4
Dose-dependent reduction of human DMPK RNA in SH-SY5Y cells by modified oligonucleotides

| Compound | DMPK RNA (% UTC) | | | | | | | | | IC₅₀ |
|---|---|---|---|---|---|---|---|---|---|---|
| No. | 3 nM | 9 nM | 27 nM | 82 nM | 247 nM | 741 nM | 2222 nM | 6667 nM | 20000 nM | (μM) |
| 598769 | 91 | 61 | 50 | 27 | 25 | 27 | 10 | 6 | 4 | 0.03 |
| 1400741 | 68 | 49 | 44 | 20 | 28 | 16 | 9 | 8 | 1 | 0.01 |
| 1522459 | 60 | 39 | 13 | 10 | 9 | 5 | 5 | 1 | 0.2 | 0.005 |
| 1522461 | 83 | 54 | 36 | 21 | 19 | 13 | 13 | 3 | 2 | 0.02 |
| 1522464 | 91 | 91 | 98 | 63 | 51 | 33 | 16 | 17 | 5 | 0.28 |
| 1525073 | 121 | 94 | 79 | 73 | 59 | 48 | 10 | 4 | 4 | 0.37 |

### Example 4: Design of Modified Oligonucleotides Complementary to Human DMPK RNA

Modified oligonucleotides complementary to a human DMPK RNA were designed as described in the table below. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (the complement of GENBANK Accession No. NT_011109.16, truncated from nucleotides 18539000 to 18566000), or to SEQ ID NO: 2 (GENBANK Accession No. NM_004409.4), or to both.

The modified oligonucleotides in the table below are 16 nucleosides in length. The sugar motifs for the modified oligonucleotides are described in the column labeled “Sugar Motif (5′ to 3′)” in the table below, wherein each “k” represents a cEt sugar moiety, each “e” represents a 2′-MOE sugar moiety, each “d” represents a 2′-β-D-deoxyribosyl sugar moiety, and each “y” represents a 2′-O-methylribosyl sugar moiety. The internucleoside linkage motif for the modified oligonucleotides is (from 5′ to 3′): soossssssssssos, wherein each “s” represents a phosphorothioate internucleoside linkage and each “o” represents a phosphodiester internucleoside linkage. Each cytosine residue is a 5-methylcytosine unless otherwise indicated. Non-methylated cytosines are represented in bold underlined italicized font as “C”.

The modified oligonucleotides in the table below are all conjugated to a THA-C₆-GalNAc₃ conjugate (designated as [THA-GalNAc-]) at the 5′ end of the modified oligonucleotide. THA-GalNAc is represented by the structure below, wherein the phosphate group is attached to the 5′-oxygen atom of the 5′-nucleoside:

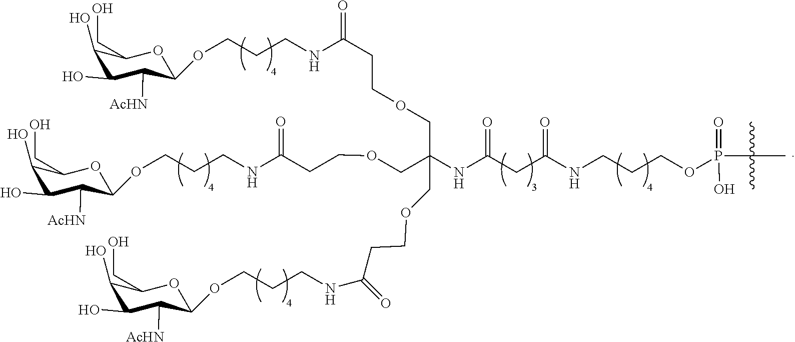

TABLE 5
THA-C6-GalNAc₃ conjugated mixed sugar
modified oligonucleotides with mixed
PS/PO internucleoside linkages
complementary to human DMPK

| | SEQ | SEQ | SEQ | SEQ | | |
|---|---|---|---|---|---|---|
| | ID | ID | ID | ID | | |
| | No: | No: | No: | No: | | |
| | 1 | 1 | 2 | 2 | Chemistry | SEQ |
| Compound | Start | Stop | Start | Stop | Notation | ID |
| Number | Site | Site | Site | Site | (5′-3′) | NO |
| 1522484 | 19498 | 19513 | 1359 | 1374 | THA-GalNAc- | 21 |
| | | | | | Tₑₛ^{m}Cₑₒ^{m}Cₖₒ^{m}Cₖₛ | |
| | | | | | G_{ds}A_{ds}A_{ds}T_{ds}G_{ds} | |
| | | | | | T_{ds}^{m}C_{ds}^{m}C_{ds}Gₖₛ | |
| | | | | | Aₖₒ^{m}CₑₛAₑ | |
| 1522487 | 19495 | 19510 | 1356 | 1371 | THA-GalNAc- | 22 |
| | | | | | ^{m}CₖₛGₖₒAₖₒA_{ds} | |
| | | | | | U_{ys}G_{ds}T_{ds}^{m}C_{ds} | |
| | | | | | ^{m}C_{ds}G_{ds}A_{ds}^{m}C_{ds} | |
| | | | | | A_{ds}GₖₒTₖₛGₖ | |
| 1522489 | 19499 | 19514 | 1360 | 1375 | THA-GalNAc- | 23 |
| | | | | | TₖₛTₖₒ^{m}Cₖₒ | |
| | | | | | ^{m}C_{ds}C_{ys}G_{ds}A_{ds} | |
| | | | | | A_{d}ST_{ds}G_{ds}T_{ds} | |
| | | | | | ^{m}C_{ds}^{m}C_{ds}GₖₒAₖₛ | |
| | | | | | ^{m}Cₖ | |
| 1522492 | 24775 | 24790 | 2833 | 2848 | THA-GalNAc- | 24 |
| | | | | | ^{m}CₖₛTₖₒTₑₚT_{ds} | |
| | | | | | T_{ds}A_{ds}T_{ds}T_{ds} | |
| | | | | | ^{m}G_{ds}G_{ds}^{m}C_{ds} | |
| | | | | | G_{ds}A_{ds}GₖₒGₖₛ | |
| | | | | | Gₖ | |
| 1525089 | 24730 | 24745 | 2788 | 2803 | THA-GalNAc- | 27 |
| | | | | | Aₖₛ^{m}CₖₒAₖₚA_{ds} | |
| | | | | | T_{ds}A_{ds}A_{ds}A_{ds} | |
| | | | | | T_{ds}A_{ds}^{m}C_{ds}^{m}C_{ds} | |
| | | | | | G_{ds}AₖₒGₖₛGₖ | |
| 1525074 | 19498 | 19513 | 1359 | 1374 | THA-GalNAc- | 25 |
| | | | | | Tₑₛ^{m}Cₑₛ^{m}Cₖₛ^{m}Cₖₛ | |
| | | | | | G_{ds}A_{ds}A_{ds}T_{ds}G_{ds} | |
| | | | | | T_{ds}^{m}C_{ds}^{m}C_{ds}Gₖₛ | |
| | | | | | Aₖₛ^{m}CₑₛAₑ | |
| 1525079 | 19499 | 19514 | 1360 | 1375 | THA-GalNAc- | 26 |
| | | | | | TₖₛTₖₛ^{m}Cₖₛ | |
| | | | | | ^{m}C_{ds}C_{ys}G_{ds}A_{ds} | |
| | | | | | A_{ds}T_{ds}G_{ds}T_{ds} | |
| | | | | | ^{m}C_{ds}^{m}C_{ds}GₖₛAₖₛ | |
| | | | | | ^{m}Cₖ | |

### Example 5: Tolerability of Modified Oligonucleotides Complementary to Human DMPK in Wildtype Mice

Wildtype BALB/c mice (Charles River Laboratory) were treated with modified oligonucleotides selected from studies described above and evaluated for changes in the levels of various plasma chemistry markers.

### Treatment

Groups of four BALB/c mice each received a single subcutaneous injection with 50 mg/kg of modified oligonucleotides. One group of four BALB/c mice received a single subcutaneous injection of PBS for each experiment. Each experiment is identified in separate tables below.

### Plasma Chemistry Markers

96 hours post treatment, mice were sacrificed. To evaluate the effect of modified oligonucleotides on liver and kidney function, plasma levels of alanine aminotransferase (ALT) and aspartate aminotransferase (AST) were measured on the day the mice were sacrificed using an automated clinical chemistry analyzer (Hitachi Olympus AU400c, Melville, NY). The results for each group of mice are presented in the tables below.

TABLE 6
Plasma chemistry markers in BALB/c mice

| Compound | | |
|---|---|---|
| No. | ALT (U/L) | AST(U/L) |
| PBS | 61 | 149 |
| 1525074 | 848 | 600 |
| 1525079 | 54 | 62 |
| 1525089 | 67 | 70 |

TABLE 7
Plasma chemistry markers in BALB/c mice

| Compound | | |
|---|---|---|
| No. | ALT (U/L) | AST(U/L) |
| PBS | 22 | 64 |
| 1522484 | 301 | 244 |
| 1522487 | 30 | 49 |
| 1522489 | 46 | 76 |
| 1522492 | 65 | 81 |

1.-30. (canceled)

31. A modified oligonucleotide according to the following chemical structure: 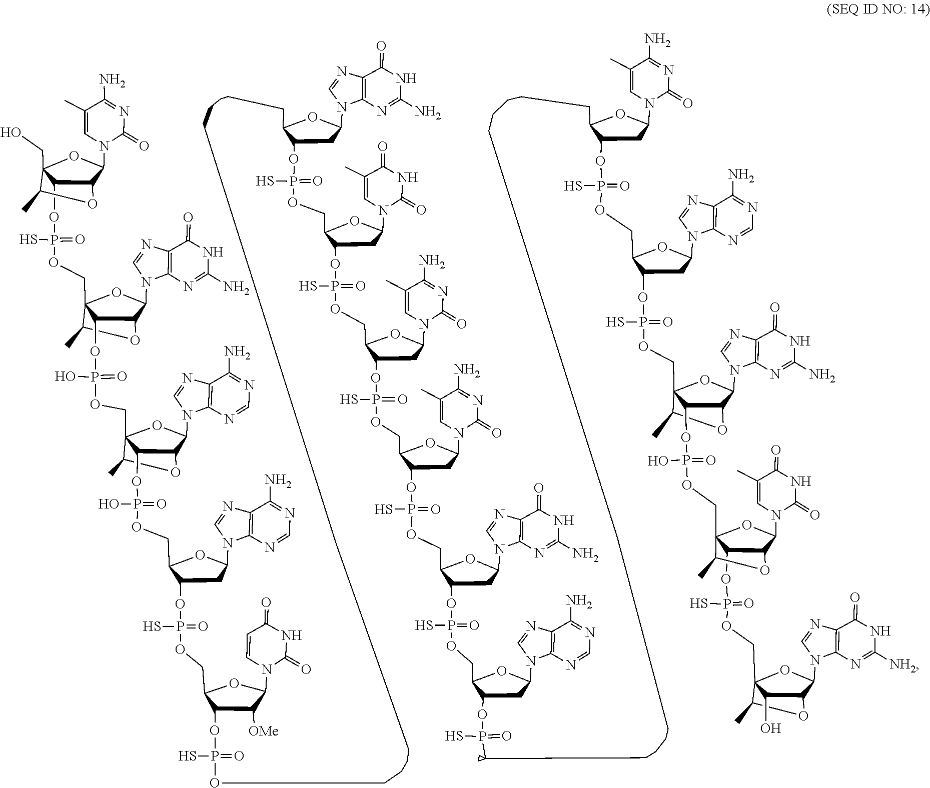
or a salt thereof.

32. The modified oligonucleotide of claim 31, which is a sodium salt or a potassium salt.

33. A modified oligonucleotide according to the following chemical structure:

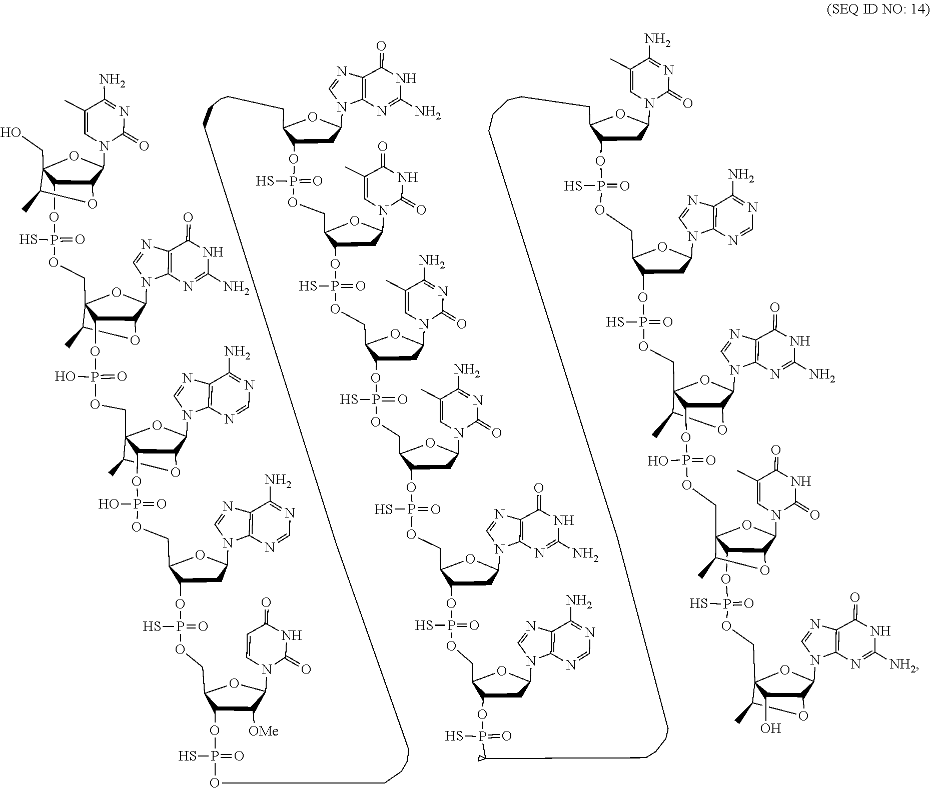

34. An oligomeric compound comprising a modified oligonucleotide according to the following chemical notation: ^{m}C_{ds}GₖₒAₖₒA_{ds}U_{ys}G_{ds}T_{ds}^{m}C_{ds}^{m}C_{ds}G_{ds}A_{ds}^{m}C_{ds}A_{ds}GₖₒTₖₛGₖ (SEQ ID NO: 14), wherein:
A=an adenine nucleobase,
^{m}C=a 5-methylcytosine nucleobase,
G=a guanine nucleobase,
T=a thymine nucleobase,
U=a uracil nucleobase,
y=a 2′-OMe sugar moiety,
k=a cEt sugar moiety,
d =a 2′-β-D-deoxyribosyl sugar moiety,
s=a phosphorothioate internucleoside linkage, and
o=a phosphodiester internucleoside linkage.

35. The oligomeric compound of claim 34 comprising a conjugate group.

36. The oligomeric compound of claim 35, wherein the conjugate group comprises a conjugate moiety and a conjugate linker.

37. The oligomeric compound of claim 36, wherein the conjugate moiety is a cell-targeting moiety.

38. The oligomeric compound of claim 37, wherein the cell-targeting moiety is selected from a carbohydrate, an antibody, and an antibody fragment.

39. The oligomeric compound of claim 38, wherein the cell-targeting moiety binds a cell surface receptor on a skeletal muscle cell.

40. An oligomeric compound according to the following chemical structure: 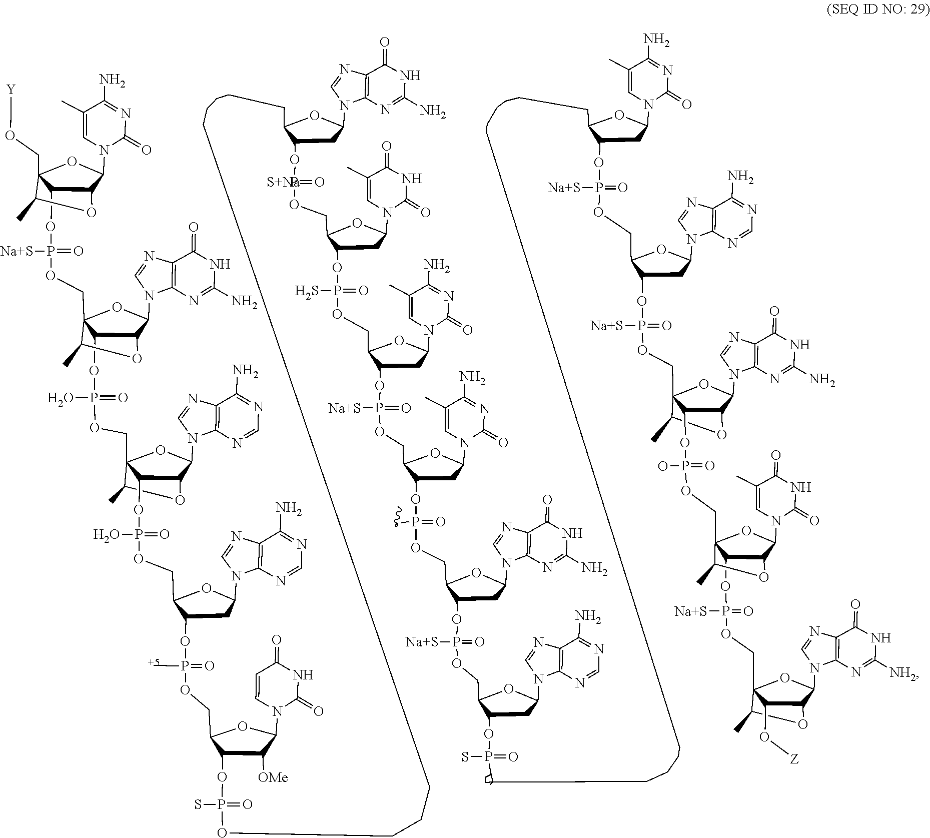
or a salt thereof, wherein Y and Z are selected from hydrogen and a conjugate group, wherein at least one of Y and Z is a conjugate group.

41. The oligomeric compound of claim 40, wherein the conjugate group comprises a conjugate moiety and a conjugate linker.

42. The oligomeric compound of claim 40, wherein the conjugate group comprises C₁₀-C₂₄ alkyl.

43. The oligomeric compound of claim 41, wherein the conjugate moiety is a cell-targeting moiety.

44. The oligomeric compound of claim 43, wherein the cell-targeting moiety binds a cell surface receptor on a skeletal muscle cell.

45. The oligomeric compound of claim 43, wherein the cell-targeting moiety is selected from a carbohydrate and an antibody.

46. The oligomeric compound of claim 43, wherein the cell-targeting moiety is an antibody or an antibody fragment that binds a transferrin receptor.

47. An oligomeric compound according to the following chemical structure: 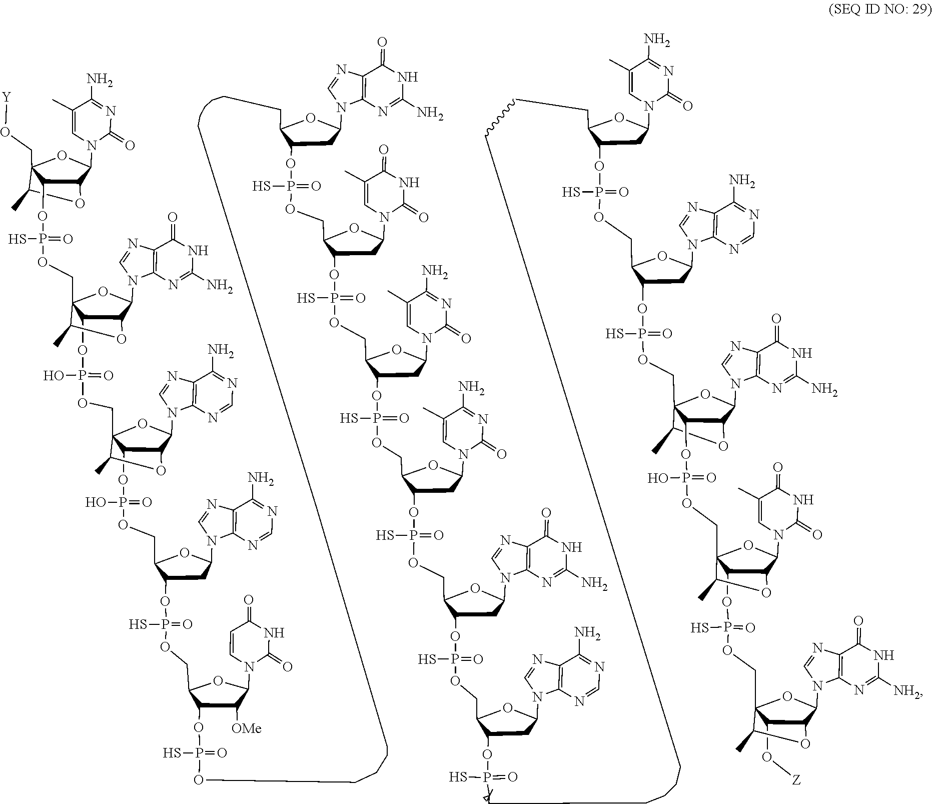
wherein Y and Z are selected from hydrogen and a conjugate group, wherein at least one of Y and Z is a conjugate group.

48. The oligomeric compound of claim 47, wherein the conjugate group comprises a conjugate moiety and a conjugate linker.

49. The oligomeric compound of claim 47, wherein the conjugate group comprises a C₁₀-C₂₄ alkyl.

50. The oligomeric compound of claim 48, wherein the conjugate moiety is a cell-targeting moiety.

51. The oligomeric compound of claim 50, wherein the cell-targeting moiety binds a cell surface receptor on a skeletal muscle cell.

52. The oligomeric compound of claim 50, wherein the cell-targeting moiety is selected from a carbohydrate and an antibody.

53. The oligomeric compound of claim 50, wherein the cell-targeting moiety is an antibody or an antibody fragment that binds a transferrin receptor.

54. A population of modified oligonucleotides of claim 31, wherein all of the phosphorothioate internucleoside linkages of the modified oligonucleotides are stereorandom.

55. A population of oligomeric compounds of claim 34, wherein all of the phosphorothioate internucleoside linkages of the oligomeric compounds are stereorandom.

56. A population of oligomeric compounds of claim 40, wherein all of the phosphorothioate internucleoside linkages of the oligomeric compounds are stereorandom.

57. A population of oligomeric compounds of claim 47, wherein all of the phosphorothioate internucleoside linkages of the oligomeric compounds are stereorandom.

58. A pharmaceutical composition comprising a modified oligonucleotide of claim 31 and a pharmaceutically acceptable diluent.

59. The pharmaceutical composition of claim 58, wherein the pharmaceutically acceptable diluent is water or phosphate-buffered saline.

60. A pharmaceutical composition comprising an oligomeric compound of claim 34 and a pharmaceutically acceptable diluent.

61. The pharmaceutical composition of claim 60, wherein the pharmaceutically acceptable diluent is water or phosphate-buffered saline.

62. A pharmaceutical composition comprising an oligomeric compound of claim 40 and a pharmaceutically acceptable diluent.

63. The pharmaceutical composition of claim 62, wherein the pharmaceutically acceptable diluent is water or phosphate-buffered saline.


SEQUENCE LISTING (37 sequences)

SEQ ID NO: 1 - DNA, 27001 bp, Homo sapiens
  source 1..27001: mol_type = genomic DNA
acttgaacccaggaggcagaggttgcagtgagccaaaatcgtcccattgcactccagcccacgtgacaagagcgagactctgtctcaaaaaaaaaaaaaaaaaaaaaaaaagccgggtgcagtggctcatgcctgtactcccagcactttgggaggccgaggtgggtggatcacctgaggtcaggagttcgagaccagactggccaacatggtgaaaccccgtctctgagccgagatggagccattgcactccagcctgggtgacagggtgagactgtctcaaaaaaaacaaaaacaaaaacaaaataaaacctctctacatgccaagtgaaaggtggaagtgggggtgagacagaaatctgggagaccaagagacacggttgatgtgggggtgggggaagggcttgggtagaggagaagcttggatcagggagaacaaggaactgagaaagaggaattccacgtggtcgaatgcaaaagacttggaggctgtaagcgggggcagagtgaagataaatggtacacatctaggaagacacccaggtctgtggcctggttgactgagtggacggcagagctagccctaaggtggggatgcaagagaggtggaggatttggtgtaagacgtcaatttcattgtaggattcttttttctttcttttttttttttttttagagagactgagtctgactctgtcccccaggctttagtgcagtgatgtgaattcatagcagtcttgatctcctgggctcaaacgatcctcccacctcagcctcccaaagtgctgggattctcggattccaggcatgagccaccatgcccgaaccattgtaggatttttttttttaagtgtgagatgcccttgggacactctagggagaaatcgtagaggccattgactacatgagactagaaagatctatgatggggctgggtggcttcagcagacatggggagcactggctatgagtaggagctgttgggatcagggggagactttgggttacgagtggatcactgggacactggggcaccgtggggatcaatacaatttgggaatcagaggtaggttttggaggacaagtgaggttgttgaaaataagaaaatcatggaaatatacattggaggttggtggaaaatatgaaggatgtttggagttagcattaatcaatagtgggtcagaggaagtcctctgagttgtgtggggacacagggatttggtgggtcaccaagggtggggaagaatggtagcagatgctgtggtcattcagggatttgaatatgtcagtgggaaacactggaggtcaggggagtgtatattgggcgtctaaggagagaccagggtcacttggggttggctgtgacttcatgggaaggctggactatggatgagggcttctttgtcgtgggggaaaaaaaaaaaaaaatccctccgttgctaggcaaacgttcccagggcgcggttgctaaggacttgagctttctaactggccacgcctctttccattgcgggctccgcccccgcagccaaccacaagtcggtcacaggctcaaaggccgcgcccaccttctcgccccgcccacagtcccagcaccgttacccgagcgaccgcggggattcgtcccgcctccggatctgggtgccaatcgaagaggcgttgcccgggcgacgaccccaccttttccggcgcgggccagcggcttccgggagggggcccggctgcgggcggcagtccggcagtggcggggcccggggggcgcccaagatggcggcgggcggcgcggagggcggctcgggccccggcgccgccatgggggactgcgcggagattaagtcgcaattccgcacgcgcgagggtttctacaagctactcccgggcgacggcgccgctcgcaggtcgggtccggcttccgcccagactccggtgccgcctcagccaccgcagcccccgcccggccctgcctccgcctccggtcccggcgctgcaggccccgcgtcgtccccgccgcccgcaggccccggacccgggcccgccctgcccgccgtgcgcctcagcctcgtgcgcctcggggagccggacagcgccggggccggggagccgcccgccacgcccgcggggctgggctcggggggagaccgcgtctgcttcaacttgggccgtgagctctatttctacccaggctgctgtcgtcgtgggagccaacgggtgagcggcctgcatcgtggggccctgggtgctgaaaggatggaggttccagagaactgataggagggtccccgaattgatatggggccctgagataggcattctaaatgtgatggacgttgtagggcgacagcattggagggagtacaggattctgaaatgtgatgagaggcccgcggggtgctgggcaggcatcttggaggtgatgggggaaatcccctttacccccgttatgcgtaggagttctgggatggtgactattggacatgatggggtatctgagatgaggtgggatcccccgggttctggggtggcaggcattctgcaggtaatggggaatcatgaggtgatgcaagcccagaggtgatggagaacccagtattgcagcctggaggtgtgggagaggggtcttgagattgagcccaggaatgaggagaagtctagaagtgatgggggccttgaaggtgataggggatccagaagctgaatgggattctagagaaaattggggtctggaagtcatgagggtcctggaggagatgagcgtccggaagctgatgggagctctcaagggaatgagaggccctggcaggggaatctgcaggtgtgattcaggacatctctaaagcgatgtggtgagaaccctggccgtttatggtgatcccagaaatgatggggatccttcggagcccctaggtaataaatgggcctaaaatgtgggagaccgtgaggtttgacgggtcccagaactgtggcggtttagggactacaaagtgaagagaatgctaggatgttaggctttagagtcctggaaatttgggtgacctcaaggtttgggggtcttggaatagtgtggtactaggtcccaagggagttgctgaggttataggatggagtggtgaggctaaaaggaatcggagcttaggaactgtgtggacccagaaccctgattatgtggacaagggtgtctctgaattagaggtgacccaactttggggaggcactgtggtcaagggggatccttaccatcttttttgctactcttgtctgtgatttgggtccccttcccgtctgtggttgccccgtccttcgcaatccacctactcacctccccacttccctctgggttctaaccctgcttcctcttagtggtttcccccaacctcaacatcctcttccttctcctcagtggcatacaccattaactccttttctcccacccctcaagtccattgacctcaacaagccaattgacaagcggatctacaagggcacccagcccacctgccacgatttcaaccagttcactgctgccaccgagaccatctcgctgctggtgggcttctcagcgggtcaagtgcagtacctggatctcatcaaaaaggacaccagcaagctgttcaatgaggaggtgattgcagcccccagagcccccaacttctctgccttctcacctacccactatatgccattaggatgcagctgcctgcggaacagtgtaggcctgagaggggcaggaccagggagagatggagaatggggtgacagcagctaatgacagctgcctttactgtacccaggccctgtgctgtgttacccttgtgctgaatactttactccttgggttttctttctttctttctttcttttatttatttattttgagatggagtctcaccctgtcacccaggctggagtgcagtggcgcgatctctgctcactgcaagatccacctcccgggttcatgccattctcctgcctcagcctcctgagtagctgggacttcaggcacctgctgccacgcccgcctaatttattgtagttttagtggagatggggtttcaccgtgttagtcaggatggtctcgatctcctgacctcgtgatctgcccacctcggcctcccaaagtggtgggattacaggtgtgagccaccatgcccggttcttttttttttttttaattgtaattgtaatttttattgtttttaattttttttttctttttaaggcggagtctcactctgtcgcccaggctggagtgcagtggtacaatctcgggtcactgcaacctccgcctcccaggttcaagcaattctcctgcctcagcctcctgagtagctgggactacaggtgcgcactatcatacctggctaatttttttttttgagatggagtctcgctctgtcgcccaggctggagtgcagtggcgcgatctcggctcactgcaacctctgcctcccgggttcacgccattcttctgtctcagcctccccagcagctgggactacaggcgcccagcaccacgcccagctaatttttttgtatttttagtagagacgggatttcaccgtgttagtcaggatagtctcgatctcctgacctcgtgatctgcccgcctcggcctcccaaagtgctggaattaaaggcgtgagccaccacacccggccaatttttgtatttttggtagagatggggtttcaccatgttggccaggctggtctcaaacacctgaccttgtgatccgcccacctcggcctcccgaagtgctgggattacaggcatgagccaccgcgcccggccattctttgagttttcatcaaccagtggatacagaagtgtgctgcccattttgcagattagagaattgaggctagtaaataagtcgggacttagttgatgcccttgctctttgagttctgggctctggaggctttggattcaaagccctgctgcaccatttactcatcatgtgactttgggaaggtgaccattctctgagcctcacttctcctcctctgcaaaatgggattccagttcctaccttgcaggaccgctgttgtagaatcatacatggagattgctcagtcctgggccaacttggcctacgaggagataccagtaaacgaccttatccgcgggcagggactgctcttggcttctgtctcattctctctggtccagaggaggtgttccataaatattctcagaatgagtgaatggctgaagggagtgaacggaacagtcagtggattaaaaattcattagctcatccattcacaaatagtttttgagcatccgttctgtgccaggcaccgttcccagcactagggatatcacatctcagggccctggacaggaggccctgcccttttggagctttcagtctagctggggagacagacagttaatcaagttaagtcttcgtgtgtcagcaggtgattaaggcctgtttagaagaagcagtggggaaggggctgagggaagcttggagagtttagtagggtggtcaggaaagccctcactaagaggtgatactgaaataaagacctgaaggaggcaggtgagaagagttgcctggggaagggcatttttttttttttttttcctgagatagggtctcactctgtcgtccatgctggagtgcagtggtacgatcttcgttcactgcaacctccgcctcccaggctcaagcagtcctcccatatcagcctcctgagtagctgggactacatgcatgcaccaccacaactggctaacttttgtattttttgtagaggcggggtctctttgtgttgcccaagctggtctcgaactcctgggctcaagctattctcctgctttggcctcccaaagcgctgggattataggcgtgagccatcacacccagccaagaaggacattctgagtatcggggccagctaggacaaatgcaggattgcacagaagccgtgtggccagtgcagttactgagggagagaagatggtagatgagttccaagaaataacggaggggctggctcatggagagtctggaaggtcacggagagattttgccttactcttagttggaagcctgtggaggagcctttctcattggggctcacctgagccctggaaggcagaagtgactagagtgattatttgttcagttctcccagggatgggaggcacctcatcctgggagagcagctccttagttgcatacactggctgcctgagagcattagggcctcaggctctggaaccctgcttgagagaggctgcgaagtgttcagagcaggacagtgccaggtgctggctgatacttcacagagcatcgctgtggcggctgcagaggacagacaggcccaggcattggagcctgaggccagtcacacagcattgcaggcttggggtggttgaatgggatggtgagaggaggtcagattctgggtgtattttttcttttttttttattttgaggtggagtctcgctctgtcgcccaggctggagtacagtggcatgatctcccctcactgcaacctccgcctcccgggttcaagcaattctgcctcagcctcctgagtagctgggactataggcgcccgccaccatgcctggctaatttttgtatttttagtagagattttgtatctctacttgtatttcaccatattggctaggctggtctggaactcctgactttgtgatccgcctgcctcagcctcccaaagtgctgggattacaggcgtgagccaccacgcccagccacttctgggtgtattttgaaggtcgagcccacaggatttgtggaaggattggatgaggaaaagaggggcagatgccgaggttttttgtctaagcacctagcgggatggggggttgtcactcatttgtccaggaattaccttcaagctgcctcctgtgtgcactgctttaaagaatagtgaaccaaacaaagatcgcttcctttggggagctgcctttccgtgaagaaatttcctacttgaagcctgacgtagtaaagatcgggggagggttagacagatacagtggtccccaaccattttggcaccagggactggtcttatggaagacagtttttccacagactgttggggggatggttttgggatgaaaccgttctgcctctgatcatcaggtgttagattctaataaggagcgcacacctagatccctcgcatgcatagttcatggtggggttcgcactcttacgaggattgaatggtgcgctgctccggtaggaggctgggctcaggctgtaatgcctgctcgcccaccactcacctcctgctgcatggcctggttcctaacaggccacggaccactactgttccatggcccggaggttgaggaccccgagatacaggacaattctgtggcaagcaggactgtcccctcgccaaagatgggacatcgaggctccttggagcaccctgtggccaccttgcagcagcctctgtttccccatgtttccatgacctggtgtccatctgtcttccccagtttgggagcttctctccgaggaggacctgggcctggtgtggcacctgctgtgtgagcggggccatgtccaacggccttcttggggactttgggtcggggagaagttctgcctgggttttactgccttctcccaaccccacactgtctcccctggcagcggttgatcgacaagaccaaggtgacatatctgaagtggctgcctgagtcggagagcctgttcctggcatcacacgccagtggccacctgtacctgtacaacgtcagccacccctgcgcctcggccccgccccagtacagcctgctgaagcagggcgagggcttctctgtctatgctgccaagagcaaggcaccccgcaacccgctggccaagtgggcggtgggtgaggggcccctcaacgagttcgccttctcgcccgatggccggcacctggcctgtgtgagccaggatggctgcctgcgcgtcttccacttcgactccatgctcctgcgtgggctcatgaagagctactttgggggcctgctgtgtgtgtgctggagccctgacggccgctacgtggtgacgggtggcgaagatgacctggtcaccgtgtggtccttcaccgagggccgcgtggtggctcgaggccatggccacaagtcctgggtcaacgctgtggcctttgacccctacaccacaagggcagaggaggcggcgacagcagccggtgctgatggggagcggagcggcgaagaggaggaggaggagcccgaggctgcgggcacaggctcggccgggggcgccccgctctctccactgcccaaggctggctccattacttaccgctttggctcggcgggccaggacacgcagttctgcctgtgggacctcactgaagacgtgctctacccgcacccccccctggcccgcacccgcaccctccctggcacacctggcaccacgccaccggccgccagcagctcgaggggtggcgagcctggcccaggccccctgcctcgctcgctgtcccgctccaacagtctcccgcacccagctggcgggggcaaggcgggcggcccgggtgtggcggcagagcctggcacaccattcagcattggccgcttcgccacgctcacactgcaggagcggcgggaccggggggcagagaaggagcacaagcgctaccacagcctgggcaacatcagccggggtggcagtggcggcagtggcagtggtggggagaagcccagcggccctgttccccgcagccgcctggaccccgccaaggtgctgggcactgcgctgtgcccgcgcatccacgaggtgcccctgctggagccccttgtgtgcaagaagatcgcccaggagcggctcacagtcctcctgttcctggaggactgcatcatcactgcctgccaggagggcctcatctgcacctgggcccggccgggcaaggcggtgagtggccccacaccagcctgccggggacctggcaggacctttcgtgggaagaggcaggcattggcagagagagggctttgttgctgtcacagcctctggctccgtggggtgaggggaagccagggaaatcttagtgtctcagtacaagacctctcagatccttagagtgagggggtctagccctaggcagcaggcagcagaaagaggggtgggtgtgagagccagctaggaattggggcatccaaggctggccgtctgaagggcagcagatgggccccacatggccaggtcttactgcctgtcactcgaaccagaatctatttctgttgaacatctgttttttaaatcgtgaaacttttttgagtacttcaggccaaaactaggggcgagctcaagcctgtgggcatggctgccagcctgggtctgggactcaggatctgagcctcctgctgaaggcacaggctgggaatcccaggcctgggttccagtcccactccctctgtgaccctggacaagtcactgccccctctgacctccaactcatcacctcttagaacagagcctgtaggatgggcagtgggtggatgtgcttgcctcctgggtgggctgtggcgttgggaaggtcatagtaggcgaatcaggcctggcatcttgtaagttcggagctcgtcttgggtgtctcagcttcttagggcttggactcagttgcccagggtcctggaggccgtggcttggttcctcagatcctcagttttggaatcgtagagtcctgagtccctagaacttgagagcacagtctgagtgactcagaggcaagagtggtgggatttggggagtctggttgagtcctaaaagagacccctctgtctccgtagttcacagacgaggagaccgaggcccagacaggggaaggaagttggcccaggtcacccagcaagtcagtggtagaggtaggactgtccctgagttctttccccagcacctcagggtccctcccaagttagaagggagctccagtttccccctcccctcccacccttacccttaccccatggtctcactcaggatccgccaaggactttgattattgcgtgaaagtgctgactgccaggacaggaagctagctaagatgcaagttcccagcctagagcagtggcctctggggggtctagggcggacccaagggcaaggccagggtggcagcagctttggggactctgggctggctccctccccttgacactggctgaagcccaggtggtctctaacccctcccatctctccctctcatcttccccagggcatctcctcccaaccaggcaactccccgagtggcacagtggtgtgaagccatggatatcgggcccccccaaccccatgcccccagcctcctagccataaccctccctgctgacctcacagatcaacgtattaacaagactaaccatgatggatggactgctccagtccccccacctgcacaaaatttgggggccccccagactggcccggacacgggcgatgtaatagcccttgtggcctcagccttgtcccccacccactgccaagtacaatgacctcttcctctgaaacatcagtgttaccctcatccctgtccccagcatgtgactggtcactcctggggagagactccccgcccctgccacaagagccccaggtctgcagtgtgcccctcagttgagtgggcagggccgggggtggtccagccctcgcccggcccccaccccagctgcccttgctattgtctgtgcttttgaagagtgttaaattatggaagcccctcaggttcctccctgtcccgcaggacctcttatttatactaaagttccctgttttctcagcgggtctgtccccttcggaggagatgatgtagaggacctgtgtgtgtactctgtggttctaggcagtccgctttccccagaggaggagtgcaggcctgctcccagcccagcgcctcccaccccttttcatagcaggaaaagccggagcccagggagggaacggacctgcgagtcacacaactggtgacccacaccagcggctggagcaggaccctcttggggagaagagcatcctgcccgcagccagggcccctcatcaaagtcctcggtgttttttaaattatcagaactgcccaggaccacgtttcccaggccctgcccagctgggactcctcggtccttgcctcctagtttctcaggcctggccctctcaaggcccaggcaccccaggccggttggaggccccgacttccactctggagaaccgtccaccctggaaagaagagctcagattcctcttggctctcggagccgcagggagtgtgtcttcccgcgccaccctccaccccccgaaatgtttctgtttctaatcccagcctgggcaggaatgtggctccccggccaggggccaaggagctattttggggtctcgtttgcccagggagggcttggctccaccactttcctcccccagcctttgggcagcaggtcacccctgttcaggctctgagggtgccccctcctggtcctgtcctcaccaccccttccccacctcctgggaaaaaaaaaaaaaaaaaaaaaaaaagctggtataaagcagagagcctgagggctaaatttaactgtccgagtcggaatccatctctgagtcacccaagaagctgccctggcctcccgtccccttcccaggcctcaacccctttctcccacccagccccaacccccagccctcaccccctagcccccagttctggagcttgtcgggagcaagggggtggttgctactgggtcactcagcctcaattggccctgtttcagcaatgggcaggttcttcttgaaattcatcacacctgtggcttcctctgtgctctacctttttattggggtgacagtgtgacagctgagattctccatgcattccccctactctagcactgaagggttctgaagggccctggaaggagggagcttggggggctggcttgtgaggggttaaggctgggaggcgggaggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtggggtgagtgcctaccctcggggctcctgcagatggggtgggggtggggcaggagacaggtctgggcacagaggcctggctgttgggggggcaggatggcaggatgggcatggggagatcctcccatcctggggctcagagtgtggacctgggccctggggcaacatttctctgtcctatgccaccactctggaggggcagagtaaggtcagcagaggctagggtggctgtgactcagagccatggcttaggagtcacagcaggctaggctgccaacagcctcccatggcctctctgcaccccgcctcagggtcagggtcagggtcatgctgggagctccctctcctaggaccctccccccaaaagtgggctctatggccctctcccctggtttcctgtggcctggggcaagccaggagggccagcatggggcagctgccaggggcgcagccgacaggcaggtgttcggcgccagcctctccagctgccccaacaggtgcccaggcactgggagggcggtgactcacgcgggccctgtgggagaaccagctttgcagacaggcgccaccagtgccccctcctctgcgatccaggagggacaactttgggttcttctgggtgtgtctccttcttttgtaggttctgcacccacccccacccccagccccaaagtctcggttcctatgagccgtgtgggtcagccaccattcccgccaccccgggtccctgcgtcctttagttctcctggcccagggcctccaaccttccagctgtcccacaaaaccccttcttgcaagggctttccagggcctggggccagggctggaaggaggatgcttccgcttctgccagctgccttgtctgcccacctcctccccaagcccaggactcgggctcactggtcactggtttctttcattcccagcaccctgcccctctggccctcatatgtctggccctcagtgactggtgtttggtttttggcctgtgtgtaacaaactgtgtgtgacacttgtttcctgtttctccgccttcccctgcttcctcttgtgtccatctctttctgacccaggcctggttcctttccctcctcctcccatttcacagatgggaaggtggaggccaagaagggccaggccattcagcctctggaaaaaccttctcccaacctcccacagcccctaatgactctcctggcctccctttagtagaggatgaagttgggttggcagggtaaactgagaccgggtggggtaggggtctggcgctcccgggaggagcactccttttgtggcccgagctgcatctcgcggcccctcccctgccaggcctggggcgggggagggggccagggttcctgctgccttaaaagggctcaatgtcttggctctctcctccctcccccgtcctcagccctggctggttcgtccctgctggcccactctcccggaaccccccggaacccctctctttcctccagaacccactgtctcctctccttccctcccctcccatacccatccctctctccatcctgcctccacttcttccacccccgggagtccaggcctccctgtccccacagtccctgagccacaagcctccaccccagctggtcccccacccaggctgcccagtttaacattcctagtcataggaccttgacttctgagaggcctgattgtcatctgtaaataaggggtaggactaaagcactcctcctggaggactgagagatgggctggaccggagcacttgagtctgggatatgtgaccatgctacctttgtctccctgtcctgttccttcccccagccccaaatccagggttttccaaagtgtggttcaagaaccacctgcatctgaatctagaggtactggatacaaccccacgtctgggccgttacccaggacattctacatgagaacgtgggggtggggccctggctgcacctgaactgtcacctggagtcagggtggaaggtggaagaactgggtcttatttccttctccccttgttctttagggtctgtccttctgcagactccgttaccccaccctaaccatcctgcacacccttggagccctctgggccaatgccctgtcccgcaaagggcttctcaggcatctcacctctatgggagggcatttttggcccccagaaccttacacggtgtttatgtggggaagcccctgggaagcagacagtcctagggtgaagctgagaggcagagagaaggggagacagacagagggtggggctttcccccttgtctccagtgccctttctggtgaccctcggttcttttcccccaccacccccccagcggagcccatcgtggtgaggcttaaggaggtccgactgcagagggacgacttcgagattctgaaggtgatcggacgcggggcgttcagcgaggtaagccgaaccgggcgggagcctgacttgactcgtggtgggcggggcataggggttggggcggggccttagaaattgatgaatgaccgagccttagaacctagggctgggctggaggcggggcttgggaccaatgggcgtggtgtggcaggtggggcggggccacggctgggtgcagaagcgggtggagttgggtctgggcgagcccttttgttttcccgccgtctccactctgtctcactatctcgacctcaggtagcggtagtgaagatgaagcagacgggccaggtgtatgccatgaagatcatgaacaagtgggacatgctgaagaggggcgaggtgaggggctgggcggacgtggggggctttgaggatccgcgccccgtctccggctgcagctcctccgggtgccctgcaggtgtcgtgcttccgtgaggagagggacgtgttggtgaatggggaccggcggtggatcacgcagctgcacttcgccttccaggatgagaactacctggtgagctccgggccggggtgactaggaagagggacaagagcccgtgctgtcactggacgaggaggtggggagaggaagctctaggattgggggtgctgcccggaaacgtctgtgggaaagtctgtgtgcggtaagagggtgtgtcaggtggatgaggggccttccctatctgagacggggatggtgtccttcactgcccgtttctggggtgatctgggggactcttataaagatgtctctgttgcggggggtctcttacctggaatgggataggtcttcaggaattctaacggggccactgcctagggaaggagtgtctgggacctattctctgggtgttgggtggcctctgggttctctttcccagaacatctcagggggagtgaatctgcccagtgacatcccaggaaagtttttttgtttgtgtttttttttgaggggcgggggcgggggccgcaggtggtctctgatttggcccggcagatctctatggttatctctgggctggggctgcaggtctctgcccaaggatggggtgtctctgggaggggttgtcccagccatccgtgatggatcagggcctcaggggactaccaaccacccatgacgaaccccttctcagtacctggtcatggagtattacgtgggcggggacctgctgacactgctgagcaagtttggggagcggattccggccgagatggcgcgcttctacctggcggagattgtcatggccatagactcggtgcaccggcttggctacgtgcacaggtgggtgcagcatggccgaggggatagcaagcttgttccctggccgggttcttggaaggtcagagcccagagaggccagggcctggagagggaccttcttggttggggcccaccggggggtgcctgggagtaggggtcagaactgtagaagccctacaggggcggaacccgaggaagtggggtcccaggtggcactgcccggaggggcggagcctggtgggaccacagaagggaggttcatttatcccacccttctcttttcctccgtgcagggacatcaaacccgacaacatcctgctggaccgctgtggccacatccgcctggccgacttcggctcttgcctcaagctgcgggcagatggaacggtgagccagtgccctggccacagagcaactggggctgctgatgagggatggaaggcacagagtgtgggagcgggactggatttggaggggaaaagaggtggtgtgacccaggcttaagtgtgcatctgtgtggcggagtattagaccaggcagagggaggggctaagcatttggggagtggttggaaggagggcccagagctggtgggcccagaggggtgggcccaagcctcgctctgctccttttggtccaggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggtgagcacggccgcagggagacctggcctctcccggtaggcgctcccaggctatcgcctcctctccctctgagcaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggtaggtacctgtggcccctgctcggctgcgggaacctccccatgctccctccataaagttggagtaaggacagtgcctaccttctggggtcctgaatcactcattccccagagcacctgctctgtgcccatctactactgaggacccagcagtgacctagacttacagtccagtgggggaacacagagcagtcttcagacagtaaggccccagagtgatcagggctgagacaatggagtgcagggggtgggggactcctgactcagcaaggaaggtcctggagggctttctggagtggggagctatctgagctgagacttggagggatgagaagcaggagaggactcctcctcccttaggccgtctctcttcaccgtgtaacaagctgtcatggcatgcttgctcggctctgggtgcccttttgctgaacaatactggggatccagcacggaccagatgagctctggtccctgccctcatccagttgcagtctagagaattagagaattatggagagtgtggcaggtgccctgaagggaagcaacaggatacaagaaaaaatgatggggccaggcacggtggctcacgcctgtaaccccagcaatttggcaggccgaagtgggtggattgcttgagcccaggagttcgagaccagcctgggcaatgtggtgagacccccgtctctacaaaaatgttttaaaaattggttgggcgtggtggcgcatgcctgtatactcagctactagggtggccgacgtgggcttgagcccaggaggtcaaggctgcagtgagctgtgattgtgccactgcactccagcctgggcaacggagagagactctgtctcaaaaataagataaactgaaattaaaaaataggctgggctggccgggcgtggtggctcacgcctgtaatctcagcactttgggaggccgaggcgggtggatcacgaggtcaggagatcgagaccatcttggctaacacggtgaaaccccatctctcctaaaaatacaaaaaattagccaggcgtggtggcgggcgcctgtagtcccagctactcaggaggctgaggcaggagaatggcgtgaacccgggaggcagagtttgcagtgagccgagatcgtgccactgcactccagcctgggcgacagagcgagactctgtctcagaaaaaaaaaaaaaaaaaaaaaaaaataggctggaccgcggccgggcgctgtggctcatgcctgtaatcccagcactttgggagtccaaggccggtgggtcatgagatcaggagttttgagactaggctggccaacacggtgaaaccccgtctctactaaaaatacaagaaaattagctgggtgtggtctcgggtgcctgtaattccagttactggggaagctgaggcaggagaattgcttgaacctgggaggcagagtttgcagtgagccaagatcatgccactacactccagtctgggtgacagagtgagactctgtctcaaaaaaaaaaaaaaaaaaaagggttgggcaaggtggttcacgcctgtaatcccagaactttgggaggctgaggcaggcagatcactggaagtcaggagttcaagaccagcctggccaacatggtgaaaccctgtgtctactaaaaatacaaaatttagccaggcttggtggcgtatgcctgtaatgccagctactcaggaggctgaggcaggagaatcgcttgattgaacctgggaggcagagtttgcagtgggctggggttgtgccactgcactctaggctgggagacagcaagactccatctaaaaaaaaaaaacagaactgggctgggcacagtggcttatatttgtaatcccagcactttgggaggctgaggttggaggactgcttgagcccagagtttgggactacaacagctgaggtaggcggatcacttgaggtcagaagatggagaccagcctggccagcgtggcgaaaccccgtctctaccaaaaatataaaaaattagccaggcgtggtagagggcgcctgtaatctcagctactcaggacgctgaggcaggagaatcgcctgaacctgggaggcggaggttgcagtgagctgagattgcaccactgcactccagcctgggtaacagagcgagactccgtatcaaagaaaaagaaaaaagaaaaaatgctggaggggccactttagataagccctgagttggggctggtttggggggaacatgtaagccaagatcaaaaagcagtgaggggcccgccctgacgactgctgctcacatctgtgtgtcttgcgcaggagacactgtcggacattcgggaaggtgcgccgctaggggtccacctgccttttgtgggctactcctactcctgcatggccctcaggtaagcactgccctggacggcctccaggggccacgaggctgcttgagcttcctgggtcctgctccttggcagccaatggagttgcaggatcagtcttggaaccttactgttttgggcccaaagactcctaagaggccagagttggaggaccttaaattttcagatctatgtacttcaaaatgttagattgaattttaaaacctcagagtcacagactgggcttcccagaatcttgtaaccattaacttttacgtctgtagtacacagagccacaggacttcagaacttggaaaatatgaagtttagacttttacaatcagttgtaaaagaatgcaaattctttgaatcagccatataacaataaggccatttaaaagtattaatttaggcgggccgcggtggctcacgcctgtaatcctagcactttgggaggccaaggcaggtggatcatgaggtcaggagatcgagaccatcctggctaacacggtgaaaccccgtctctactaaaaatacaaaaaaattagccgggcatggtggcgggcgcttgcggtcccagctacttgggaggcgaggcaggagaatggcatgaacccgggaggcggagcttgcagtgagccgagatcatgccactgcactccagcctgggcgacagagcaagactccgtctcaaaaaaaaaaaaaaaaaagtatttatttaggccgggtgtggtggctcacgcctgtaattccagtgctttgggaggatgaggtgggtggatcacctgaggtcaggagttcgagaccagcctgaccaacgtggagaaacctcatctctactaaaaaacaaaattagccaggcgtggtggcatatacctgtaatcccagctactcaggaggctgaggcaggagaatcagaacccaggagggggaggttgtggtgagctgagatcgtgccattgcattccagcctgggcaacaagagtgaaacttcatctcaaaaaaaaaaaaaaaaaagtactaatttacaggctgggcatggtggctcacgcttggaatcccagcactttgggaggctgaagtggacggattgcttcagcccaggagttcaagaccagcctgagcaacataatgagaccctgtctctacaaaaaattgaaaaaatcgtgccaggcatggtggtctgtgcctgcagtcctagctactcaggagtctgaagtaggagaatcacttgagcctggagtttgaggcttcagtgagccatgatagattccagcctaggcaacaaagtgagacctggtctcaacaaaagtattaattacacaaataatgcattgcttatcacaagtaaattagaaaatacagataaggaaaaggaagttgatatctcgtgagctcaccagatggcagtggtccctggctcacacgtgtactgacacatgtttaaatagtggagaacaggtgtttttttggtttgtttttttccccttcctcatgctactttgtctaagagaacagttggttttctagtcagcttttattactggacaacattacacatactataccttatcattaatgaactccagcttgattctgaaccgctgcggggcctgaacggtgggtcaggattgaacccatcctctattagaacccaggcgcatgtccaggatagctaggtcctgagccgtgttcccacaggagggactgctgggttggaggggacagccacttcataccccagggaggagctgtccccttcccacagctgagtggggtgtgctgacctcaagttgccatcttggggtcccatgcccagtcttaggaccacatctgtggaggtggccagagccaagcagtctccccatcaggtcggcctccctgtcctgaggccctgagaagaggggtctgcagcggtcacatgtcaagggaggagatgagctgaccctagaacatgggggtctggaccccaagtccctgcagaaggtttagaaagagcagctcccaggggcccaaggccaggagaggggcagggcttttcctaagcagaggaggggctattggcctacctgggactctgttctcttcgctctgctgctccccttcctcaaatcaggaggtcttggaagcagctgcccctacccacaggccagaagttctggttctccaccagagaatcagcattctgtctccctccccactccctcctcctctccccagggacagtgaggtcccaggccccacacccatggaactggaggccgagcagctgcttgagccacacgtgcaagcgcccagcctggagccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaagctcagtctcggggcggaggcttcgcgcccttagtcctcctggacggcccgttaccttctgcgttgtcccgatggggaaactgaggccctgagccagaagcacacgctggggggaggcagaaagcgcggccagaggcggagggaaaacaaagggagaatcacagacagacgggagggggacggacacacacaaggggacagagacccgagtggagagctggatctcgccttcccggcgtggggcgcagggtcggccagaaagaagatcgagaagagcggggagtgggggcgaaaaggggggacaggtgggggaggaggctggggaaagcccgagggaggaagagagggagggaggaacttcccaaagttgcaaaacatggctaccttgcctgcggagccgagcgcggggccggcggctgggggggaggcggtggcggcggcggcggcgaccgaagaggaggaggaggaagcgcgccagctcttgcagactttgcaggcggccgagggtgaggcggcggcggcggccggggccggggcgggcgcagcggctgcgggagctgagggcccgggatccccgggcgtccccgggtcgccccccgaggccgcttccgaaccgcccacgggcctccgcttctcgcccgagcaggtggcgtgcgtctgcgaggcgctgctccaggcgggccacgccggccgcttgagccgcttcctgggcgcactgcccccggccgagcgcctacgtggcagcgacccggtgttgcgcgcgcgggccctggtggccttccagcggggcgagtacgccgagctctaccggctactcgagagccgccccttccccgccgcccaccacgccttcctgcaggacctctacctgcgcgcgcgctaccatgaggccgagcgggcccgcggccgcgcgcttggcgcagtggacaagtatcgactgcgcaagaagttcccgctgcccaagaccatctgggacggcgaggagacagtctactgcttcaaggagcgctcccgcgcagcgctcaaggcctgctaccgcggcaaccgctaccccacgccggacgagaagcgccgcctggccacactcaccggcctgtcgctcacgcaggtcagcaactggttcaagaaccggcgacagcgcgaccggaccggggccggaggcggcgcgccctgcaagaggtgaggggcctcgggcggcgcaagtccagctctcccggggacatcccgtccaccagccctcttcccccgtgcccactgctggggccggcgcgccgaggtcctcggacatctcccgggaccagctcacaatctcaggcgcccgcggggcgcggggactaagtgtggacgggacaggcacccgcccgggccctctccccgcacgcgtctcctcttccagcggctccattccgagctccttcccaaatcccatcggtgttggggaatcacactgcggggggcactagagggactgaggaaaaaggacagggcctgtggccactccactccgcgtaggggctgcctcccagccccctttcttcactgaccgttactcttacccacccgcccccacactccctccgggcccggacggcgtggtccgtcttctgcctttgttgtgaaacgtgaacctttcccagctccggtttccctgtaacccaaacccttctcctcccaccctgagcgccggcctctccctt

SEQ ID NO: 2 - DNA, 2874 bp, Homo sapiens
  source 1..2874: mol_type = genomic DNA
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctgga

SEQ ID NO: 3 - DNA, 19000 bp, Homo sapiens
  source 1..19000: mol_type = genomic DNA
cagctggcgggggcaaggcgggcggcccgggtgtggcggcagagcctggcacaccattcagcattggccgcttcgccacgctcacactgcaggagcggcgggaccggggggcagagaaggagcacaagcgctaccacagcctgggcaacatcagccggggtggcagtggcggcagtggcagtggtggggagaagcccagcggccctgttccccgcagccgcctggaccccgccaaggtgctgggcactgcgctgtgcccgcgcatccacgaggtgcccctgctggagccccttgtgtgcaagaagatcgcccaggagcggctcacagtcctcctgttcctggaggactgcatcatcactgcctgccaggagggcctcatctgcacctgggcccggccgggcaaggcggtgagtggccccacaccagcctgccggggacctggcaggacctttcgtgggaagaggcaggcattggcagagagagggctttgttgctgtcacagcctctggctccgtggggtgaggggaagccagggaaatcttagtgtctcagtacaagacctctcagatccttagagtgagggggtctagccctaggcagcaggcagcagaaagaggggtgggtgtgagagccagctaggaattggggcatccaaggctggccgtctgaagggcagcagatgggccccacatggccaggtcttactgcctgtcactcgaaccagaatctatttctgttgaacatctgttttttaaatcgtgaaacttttttgagtacttcaggccaaaactaggggcgagctcaagcctgtgggcatggctgccagcctgggtctgggactcaggatctgagcctcctgctgaaggcacaggctgggaatcccaggcctgggttccagtcccactccctctgtgaccctggacaagtcactgccccctctgacctccaactcatcacctcttagaacagagcctgtaggatgggcagtgggtggatgtgcttgcctcctgggtgggctgtggcgttgggaaggtcatagtaggcgaatcaggcctggcatcttgtaagttcggagctcgtcttgggtgtctcagcttcttagggcttggactcagttgcccagggtcctggaggccgtggcttggttcctcagatcctcagttttggaatcgtagagtcctgagtccctagaacttgagagcacagtctgagtgactcagaggcaagagtggtgggatttggggagtctggttgagtcctaaaagagacccctctgtctccgtagttcacagacgaggagaccgaggcccagacaggggaaggaagttggcccaggtcacccagcaagtcagtggtagaggtaggactgtccctgagttctttccccagcacctcagggtccctcccaagttagaagggagctccagtttccccctcccctcccacccttacccttaccccatggtctcactcaggatccgccaaggactttgattattgcgtgaaagtgctgactgccaggacaggaagctagctaagatgcaagttcccagcctagagcagtggcctctggggggtctagggcggacccaagggcaaggccagggtggcagcagctttggggactctgggctggctccctccccttgacactggctgaagcccaggtggtctctaacccctcccatctctccctctcatcttccccagggcatctcctcccaaccaggcaactccccgagtggcacagtggtgtgaagccatggatatcgggcccccccaaccccatgcccccagcctcctagccataaccctccctgctgacctcacagatcaacgtattaacaagactaaccatgatggatggactgctccagtccccccacctgcacaaaatttgggggccccccagactggcccggacacgggcgatgtaatagcccttgtggcctcagccttgtcccccacccactgccaagtacaatgacctcttcctctgaaacatcagtgttaccctcatccctgtccccagcatgtgactggtcactcctggggagagactccccgcccctgccacaagagccccaggtctgcagtgtgcccctcagttgagtgggcagggccgggggtggtccagccctcgcccggcccccaccccagctgcccttgctattgtctgtgcttttgaagagtgttaaattatggaagcccctcaggttcctccctgtcccgcaggacctcttatttatactaaagttccctgttttctcagcgggtctgtccccttcggaggagatgatgtagaggacctgtgtgtgtactctgtggttctaggcagtccgctttccccagaggaggagtgcaggcctgctcccagcccagcgcctcccaccccttttcatagcaggaaaagccggagcccagggagggaacggacctgcgagtcacacaactggtgacccacaccagcggctggagcaggaccctcttggggagaagagcatcctgcccgcagccagggcccctcatcaaagtcctcggtgttttttaaattatcagaactgcccaggaccacgtttcccaggccctgcccagctgggactcctcggtccttgcctcctagtttctcaggcctggccctctcaaggcccaggcaccccaggccggttggaggccccgacttccactctggagaaccgtccaccctggaaagaagagctcagattcctcttggctctcggagccgcagggagtgtgtcttcccgcgccaccctccaccccccgaaatgtttctgtttctaatcccagcctgggcaggaatgtggctccccggccaggggccaaggagctattttggggtctcgtttgcccagggagggcttggctccaccactttcctcccccagcctttgggcagcaggtcacccctgttcaggctctgagggtgccccctcctggtcctgtcctcaccaccccttccccacctcctgggaaaaaaaaaaaaaaaaaaaaaaaaagctggtataaagcagagagcctgagggctaaatttaactgtccgagtcggaatccatctctgagtcacccaagaagctgccctggcctcccgtccccttcccaggcctcaacccctttctcccacccagccccaacccccagccctcaccccctagcccccagttctggagcttgtcgggagcaagggggtggttgctactgggtcactcagcctcaattggccctgtttcagcaatgggcaggttcttcttgaaattcatcacacctgtggcttcctctgtgctctacctttttattggggtgacagtgtgacagctgagattctccatgcattccccctactctagcactgaagggttctgaagggccctggaaggagggagcttggggggctggcttgtgaggggttaaggctgggaggcgggaggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtggggtgagtgcctaccctcggggctcctgcagatggggtgggggtggggcaggagacaggtctgggcacagaggcctggctgttgggggggcaggatggcaggatgggcatggggagatcctcccatcctggggctcagagtgtggacctgggccctggggcaacatttctctgtcctatgccaccactctggaggggcagagtaaggtcagcagaggctagggtggctgtgactcagagccatggcttaggagtcacagcaggctaggctgccaacagcctcccatggcctctctgcaccccgcctcagggtcagggtcagggtcatgctgggagctccctctcctaggaccctccccccaaaagtgggctctatggccctctcccctggtttcctgtggcctggggcaagccaggagggccagcatggggcagctgccaggggcgcagccgacaggcaggtgttcggcgccagcctctccagctgccccaacaggtgcccaggcactgggagggcggtgactcacgcgggccctgtgggagaaccagctttgcagacaggcgccaccagtgccccctcctctgcgatccaggagggacaactttgggttcttctgggtgtgtctccttcttttgtaggttctgcacccacccccacccccagccccaaagtctcggttcctatgagccgtgtgggtcagccaccattcccgccaccccgggtccctgcgtcctttagttctcctggcccagggcctccaaccttccagctgtcccacaaaaccccttcttgcaagggctttccagggcctggggccagggctggaaggaggatgcttccgcttctgccagctgccttgtctgcccacctcctccccaagcccaggactcgggctcactggtcactggtttctttcattcccagcaccctgcccctctggccctcatatgtctggccctcagtgactggtgtttggtttttggcctgtgtgtaacaaactgtgtgtgacacttgtttcctgtttctccgccttcccctgcttcctcttgtgtccatctctttctgacccaggcctggttcctttccctcctcctcccatttcacagatgggaaggtggaggccaagaagggccaggccattcagcctctggaaaaaccttctcccaacctcccacagcccctaatgactctcctggcctccctttagtagaggatgaagttgggttggcagggtaaactgagaccgggtggggtaggggtctggcgctcccgggaggagcactccttttgtggcccgagctgcatctcgcggcccctcccctgccaggcctggggcgggggagggggccagggttcctgctgccttaaaagggctcaatgtcttggctctctcctccctcccccgtcctcagccctggctggttcgtccctgctggcccactctcccggaaccccccggaacccctctctttcctccagaacccactgtctcctctccttccctcccctcccatacccatccctctctccatcctgcctccacttcttccacccccgggagtccaggcctccctgtccccacagtccctgagccacaagcctccaccccagctggtcccccacccaggctgcccagtttaacattcctagtcataggaccttgacttctgagaggcctgattgtcatctgtaaataaggggtaggactaaagcactcctcctggaggactgagagatgggctggaccggagcacttgagtctgggatatgtgaccatgctacctttgtctccctgtcctgttccttcccccagccccaaatccagggttttccaaagtgtggttcaagaaccacctgcatctgaatctagaggtactggatacaaccccacgtctgggccgttacccaggacattctacatgagaacgtgggggtggggccctggctgcacctgaactgtcacctggagtcagggtggaaggtggaagaactgggtcttatttccttctccccttgttctttagggtctgtccttctgcagactccgttaccccaccctaaccatcctgcacacccttggagccctctgggccaatgccctgtcccgcaaagggcttctcaggcatctcacctctatgggagggcatttttggcccccagaaccttacacggtgtttatgtggggaagcccctgggaagcagacagtcctagggtgaagctgagaggcagagagaaggggagacagacagagggtggggctttcccccttgtctccagtgccctttctggtgaccctcggttcttttcccccaccacccccccagcggagcccatcgtggtgaggcttaaggaggtccgactgcagagggacgacttcgagattctgaaggtgatcggacgcggggcgttcagcgaggtaagccgaaccgggcgggagcctgacttgactcgtggtgggcggggcataggggttggggcggggccttagaaattgatgaatgaccgagccttagaacctagggctgggctggaggcggggcttgggaccaatgggcgtggtgtggcaggtggggcggggccacggctgggtgcagaagcgggtggagttgggtctgggcgagcccttttgttttcccgccgtctccactctgtctcactatctcgacctcaggtagcggtagtgaagatgaagcagacgggccaggtgtatgccatgaagatcatgaacaagtgggacatgctgaagaggggcgaggtgaggggctgggcggacgtggggggctttgaggatccgcgccccgtctccggctgcagctcctccgggtgccctgcaggtgtcgtgcttccgtgaggagagggacgtgttggtgaatggggaccggcggtggatcacgcagctgcacttcgccttccaggatgagaactacctggtgagctccgggccggggtgactaggaagagggacaagagcccgtgctgtcactggacgaggaggtggggagaggaagctctaggattgggggtgctgcccggaaacgtctgtgggaaagtctgtgtgcggtaagagggtgtgtcaggtggatgaggggccttccctatctgagacggggatggtgtccttcactgcccgtttctggggtgatctgggggactcttataaagatgtctctgttgcggggggtctcttacctggaatgggataggtcttcaggaattctaacggggccactgcctagggaaggagtgtctgggacctattctctgggtgttgggtggcctctgggttctctttcccagaacatctcagggggagtgaatctgcccagtgacatcccaggaaagtttttttgtttgtgtttttttttgaggggcgggggcgggggccgcaggtggtctctgatttggcccggcagatctctatggttatctctgggctggggctgcaggtctctgcccaaggatggggtgtctctgggaggggttgtcccagccatccgtgatggatcagggcctcaggggactaccaaccacccatgacgaaccccttctcagtacctggtcatggagtattacgtgggcggggacctgctgacactgctgagcaagtttggggagcggattccggccgagatggcgcgcttctacctggcggagattgtcatggccatagactcggtgcaccggcttggctacgtgcacaggtgggtgcagcatggccgaggggatagcaagcttgttccctggccgggttcttggaaggtcagagcccagagaggccagggcctggagagggaccttcttggttggggcccaccggggggtgcctgggagtaggggtcagaactgtagaagccctacaggggcggaacccgaggaagtggggtcccaggtggcactgcccggaggggcggagcctggtgggaccacagaagggaggttcatttatcccacccttctcttttcctccgtgcagggacatcaaacccgacaacatcctgctggaccgctgtggccacatccgcctggccgacttcggctcttgcctcaagctgcgggcagatggaacggtgagccagtgccctggccacagagcaactggggctgctgatgagggatggaaggcacagagtgtgggagcgggactggatttggaggggaaaagaggtggtgtgacccaggcttaagtgtgcatctgtgtggcggagtattagaccaggcagagggaggggctaagcatttggggagtggttggaaggagggcccagagctggtgggcccagaggggtgggcccaagcctcgctctgctccttttggtccaggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggtgagcacggccgcagggagacctggcctctcccggtaggcgctcccaggctatcgcctcctctccctctgagcaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggtaggtacctgtggcccctgctcggctgcgggaacctccccatgctccctccataaagttggagtaaggacagtgcctaccttctggggtcctgaatcactcattccccagagcacctgctctgtgcccatctactactgaggacccagcagtgacctagacttacagtccagtgggggaacacagagcagtcttcagacagtaaggccccagagtgatcagggctgagacaatggagtgcagggggtgggggactcctgactcagcaaggaaggtcctggagggctttctggagtggggagctatctgagctgagacttggagggatgagaagcaggagaggactcctcctcccttaggccgtctctcttcaccgtgtaacaagctgtcatggcatgcttgctcggctctgggtgcccttttgctgaacaatactggggatccagcacggaccagatgagctctggtccctgccctcatccagttgcagtctagagaattagagaattatggagagtgtggcaggtgccctgaagggaagcaacaggatacaagaaaaaatgatggggccaggcacggtggctcacgcctgtaaccccagcaatttggcaggccgaagtgggtggattgcttgagcccaggagttcgagaccagcctgggcaatgtggtgagacccccgtctctacaaaaatgttttaaaaattggttgggcgtggtggcgcatgcctgtatactcagctactagggtggccgacgtgggcttgagcccaggaggtcaaggctgcagtgagctgtgattgtgccactgcactccagcctgggcaacggagagagactctgtctcaaaaataagataaactgaaattaaaaaataggctgggctggccgggcgtggtggctcacgcctgtaatctcagcactttgggaggccgaggcgggtggatcacgaggtcaggagatcgagaccatcttggctaacacggtgaaaccccatctctcctaaaaatacaaaaaattagccaggcgtggtggcgggcgcctgtagtcccagctactcaggaggctgaggcaggagaatggcgtgaacccgggaggcagagtttgcagtgagccgagatcgtgccactgcactccagcctgggcgacagagcgagactctgtctcagaaaaaaaaaaaaaaaaaaaaaaaaataggctggaccgcggccgggcgctgtggctcatgcctgtaatcccagcactttgggagtccaaggccggtgggtcatgagatcaggagttttgagactaggctggccaacacggtgaaaccccgtctctactaaaaatacaagaaaattagctgggtgtggtctcgggtgcctgtaattccagttactggggaagctgaggcaggagaattgcttgaacctgggaggcagagtttgcagtgagccaagatcatgccactacactccagtctgggtgacagagtgagactctgtctcaaaaaaaaaaaaaaaaaaaagggttgggcaaggtggttcacgcctgtaatcccagaactttgggaggctgaggcaggcagatcactggaagtcaggagttcaagaccagcctggccaacatggtgaaaccctgtgtctactaaaaatacaaaatttagccaggcttggtggcgtatgcctgtaatgccagctactcaggaggctgaggcaggagaatcgcttgattgaacctgggaggcagagtttgcagtgggctggggttgtgccactgcactctaggctgggagacagcaagactccatctaaaaaaaaaaaacagaactgggctgggcacagtggcttatatttgtaatcccagcactttgggaggctgaggttggaggactgcttgagcccagagtttgggactacaacagctgaggtaggcggatcacttgaggtcagaagatggagaccagcctggccagcgtggcgaaaccccgtctctaccaaaaatataaaaaattagccaggcgtggtagagggcgcctgtaatctcagctactcaggacgctgaggcaggagaatcgcctgaacctgggaggcggaggttgcagtgagctgagattgcaccactgcactccagcctgggtaacagagcgagactccgtatcaaagaaaaagaaaaaagaaaaaatgctggaggggccactttagataagccctgagttggggctggtttggggggaacatgtaagccaagatcaaaaagcagtgaggggcccgccctgacgactgctgctcacatctgtgtgtcttgcgcaggagacactgtcggacattcgggaaggtgcgccgctaggggtccacctgccttttgtgggctactcctactcctgcatggccctcaggtaagcactgccctggacggcctccaggggccacgaggctgcttgagcttcctgggtcctgctccttggcagccaatggagttgcaggatcagtcttggaaccttactgttttgggcccaaagactcctaagaggccagagttggaggaccttaaattttcagatctatgtacttcaaaatgttagattgaattttaaaacctcagagtcacagactgggcttcccagaatcttgtaaccattaacttttacgtctgtagtacacagagccacaggacttcagaacttggaaaatatgaagtttagacttttacaatcagttgtaaaagaatgcaaattctttgaatcagccatataacaataaggccatttaaaagtattaatttaggcgggccgcggtggctcacgcctgtaatcctagcactttgggaggccaaggcaggtggatcatgaggtcaggagatcgagaccatcctggctaacacggtgaaaccccgtctctactaaaaatacaaaaaaattagccgggcatggtggcgggcgcttgcggtcccagctacttgggaggcgaggcaggagaatggcatgaacccgggaggcggagcttgcagtgagccgagatcatgccactgcactccagcctgggcgacagagcaagactccgtctcaaaaaaaaaaaaaaaaaagtatttatttaggccgggtgtggtggctcacgcctgtaattccagtgctttgggaggatgaggtgggtggatcacctgaggtcaggagttcgagaccagcctgaccaacgtggagaaacctcatctctactaaaaaacaaaattagccaggcgtggtggcatatacctgtaatcccagctactcaggaggctgaggcaggagaatcagaacccaggagggggaggttgtggtgagctgagatcgtgccattgcattccagcctgggcaacaagagtgaaacttcatctcaaaaaaaaaaaaaaaaaagtactaatttacaggctgggcatggtggctcacgcttggaatcccagcactttgggaggctgaagtggacggattgcttcagcccaggagttcaagaccagcctgagcaacataatgagaccctgtctctacaaaaaattgaaaaaatcgtgccaggcatggtggtctgtgcctgcagtcctagctactcaggagtctgaagtaggagaatcacttgagcctggagtttgaggcttcagtgagccatgatagattccagcctaggcaacaaagtgagacctggtctcaacaaaagtattaattacacaaataatgcattgcttatcacaagtaaattagaaaatacagataaggaaaaggaagttgatatctcgtgagctcaccagatggcagtggtccctggctcacacgtgtactgacacatgtttaaatagtggagaacaggtgtttttttggtttgtttttttccccttcctcatgctactttgtctaagagaacagttggttttctagtcagcttttattactggacaacattacacatactataccttatcattaatgaactccagcttgattctgaaccgctgcggggcctgaacggtgggtcaggattgaacccatcctctattagaacccaggcgcatgtccaggatagctaggtcctgagccgtgttcccacaggagggactgctgggttggaggggacagccacttcataccccagggaggagctgtccccttcccacagctgagtggggtgtgctgacctcaagttgccatcttggggtcccatgcccagtcttaggaccacatctgtggaggtggccagagccaagcagtctccccatcaggtcggcctccctgtcctgaggccctgagaagaggggtctgcagcggtcacatgtcaagggaggagatgagctgaccctagaacatgggggtctggaccccaagtccctgcagaaggtttagaaagagcagctcccaggggcccaaggccaggagaggggcagggcttttcctaagcagaggaggggctattggcctacctgggactctgttctcttcgctctgctgctccccttcctcaaatcaggaggtcttggaagcagctgcccctacccacaggccagaagttctggttctccaccagagaatcagcattctgtctccctccccactccctcctcctctccccagggacagtgaggtcccaggccccacacccatggaactggaggccgagcagctgcttgagccacacgtgcaagcgcccagcctggagccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaagctcagtctcggggcggaggcttcgcgcccttagtcctcctggacggcccgttaccttctgcgttgtcccgatggggaaactgaggccctgagccagaagcacacgctggggggaggcagaaagcgcggccagaggcggagggaaaacaaagggagaatcacagacagacgggagggggacggacacacacaaggggacagagacccgagtggagagctggatctcgccttcccggcgtggggcgcagggtcggccagaaagaagatcgagaagagcggggagtgggggcgaaaaggggggacaggtgggggaggaggctggggaaagcccgagggaggaagagagggagggaggaacttcccaaagttgcaaaacatggctaccttgcctgcggagccgagcgcggggccggcggctgggggggaggcggtggcggcggcggcggcgaccgaagaggaggaggaggaagcgcgccagctcttgcagactttgcaggcggccgagggtgaggcggcggcggcggccggggccggggcgggcgcagcggctgcgggagctgagggcccgggatccccgggcgtccccgggtcgccccccgaggccgcttccgaaccgcccacgggcctccgcttctcgcccgagcaggtggcgtgcgtctgcgaggcgctgctccaggcgggccacgccggccgcttgagccgcttcctgggcgcactgcccccggccgagcgcctacgtggcagcgacccggtgttgcgcgcgcgggccctggtggccttccagcggggcgagtacgccgagctctaccggctactcgagagccgccccttccccgccgcccaccacgccttcctgcaggacctctacctgcgcgcgcgctaccatgaggccgagcgggcccgcggccgcgcgcttggcgcagtggacaagtatcgactgcgcaagaagttcccgctgcccaagaccatctgggacggcgaggagacagtctactgcttcaaggagcgctcccgcgcagcgctcaaggcctgctaccgcggcaaccgctaccccacgccggacgagaagcgccgcctggccacactcaccggcctgtcgctcacgcaggtcagcaactggttcaagaaccggcgacagcgcgaccggaccggggccggaggcggcgcgccctgcaagaggtgaggggcctcgggcggcgcaagtccagctctcccggggacatcccgtccaccagccctcttcccccgtgcccactgctggggccggcgcgccgaggtcctcggacatctcccgggaccagctcacaatctcaggcgcccgcggggcgcggggactaagtgtggacgggacaggcacccgcccgggccctctccccgcacgcgtctcctcttccagcggctccattccgagctccttcccaaatcccatcggtgttggggaatcacactgcggggggcactagagggactgaggaaaaaggacagggcctgtggccactccactccgcgtaggggctgcctcccagccccctttcttcactgaccgttactcttacccacccgcccccacactccctccgggcccggacggcgtggtccgtcttctgcctttgttgtgaaacgtgaacctttcccagctccggtttccctgtaacccaaacccttctcctcccaccctgagcgccggcctctcccttctctgacgccccgcggggagggcaccgggaactgggctggcggctgctgctccctctctccaggccactcgcccgctttccgcccaaccaaggaacttttagggcaacctgggagctcagcctctggggacccgaggccgaggtgggggagagcaagggcctcttccctccccagcgcttctctgcagtggtgtccacgcagggcttgcaaaaggccctggagtttggagtggtctcagagaggcgtcataacccctggaatgatccaccgaaacttgggccagggaaaggtgggggagcagctggctggctatgcgggaggccccacaggctaagggaccccagctgacagccatttctccggccagcgagtctgatgggaatcccacgactgaggacgagtccagccgaagtcctgaggacctggagagaggggcggccccagtgtccgccgaggccgctgcccagggctccatattcctggcagggaccggccctcccgcgccttgcccggcttcctcct

SEQ ID NO: 4 - DNA, 2933 bp, Homo sapiens
  source 1..2933: mol_type = genomic DNA
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggccccaaatccagggttttccaaagtgtggttcaagaaccacctgcatctgaatctagagcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctgga

SEQ ID NO: 5 - DNA, 2859 bp, Homo sapiens
  source 1..2859: mol_type = genomic DNA
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctgga

SEQ ID NO: 6 - DNA, 17 bp, synthetic construct
  source 1..17: mol_type = other DNA
ctgagccgggagatgga

SEQ ID NO: 7 - DNA, 18 bp, synthetic construct
  source 1..18: mol_type = other DNA
ggacgtgtgcctctaggt

SEQ ID NO: 8 - DNA, 24 bp, synthetic construct
  source 1..24: mol_type = other DNA
tgactggcgaagttctggttgtcc

SEQ ID NO: 9-10 - skipped (2 entries)

SEQ ID NO: 11 - DNA, 16 bp, synthetic construct
  source 1..16: mol_type = other DNA
  modified_base 1: mod_base = OTHER; note = constrained ethyl adenosine 3'-phosphorothioate
  modified_base 2: mod_base = OTHER; note = constrained ethyl 5-methylcytidine 3'-phosphodiester
  modified_base 3: mod_base = OTHER; note = constrained ethyl adenosine 3'-phosphodiester
  modified_base 4: mod_base = OTHER; note = 2'-deoxyadenosine 3'-phosphorothioate
  modified_base 5: mod_base = OTHER; note = 2'-deoxythymidine 3'-phosphorothioate
  modified_base 6..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyadenosine 3'-phosphorothioate
  modified_base 11..12: mod_base = OTHER; note = 5-methyl-2'-deoxycytidine 3'-phosphorothioate
  modified_base 13: mod_base = OTHER; note = 2'-deoxyguanosine 3'-phosphorothioate
  modified_base 14: mod_base = OTHER; note = constrained ethyl adenosine 3'-phosphodiester
  modified_base 15: mod_base = OTHER; note = constrained ethyl guanosine 3'-phosphorothioate
  modified_base 16: mod_base = OTHER; note = constrained ethyl guanosine
acaataaataccgagg

SEQ ID NO: 12 - DNA, 16 bp, synthetic construct
  source 1..16: mol_type = other DNA
  modified_base 1: mod_base = OTHER; note = 2'-O-methoxyethyl thymidine 3'-phosphorothioate
  modified_base 2: mod_base = OTHER; note = 2'-O-methoxyethyl 5-methylcytidine 3'-phosphorothioate
  modified_base 3..4: mod_base = OTHER; note = constrained ethyl 5-methylcytidine 3'-phosphorothioate
  modified_base 5: mod_base = OTHER; note = 2'-deoxyguanosine 3'-phosphorothioate
  modified_base 6..7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 2'-deoxyguanosine 3'-phosphorothioate
  modified_base 10: mod_base = OTHER; note = 2'-deoxythymidine 3'-phosphorothioate
  modified_base 11..12: mod_base = OTHER; note = 5-methyl-2'-deoxycytidine 3'-phosphorothioate
  modified_base 13: mod_base = OTHER; note = constrained ethyl guanosine 3'-phosphorothioate
  modified_base 14: mod_base = OTHER; note = constrained ethyl adenosine 3'-phosphorothioate
  modified_base 15: mod_base = OTHER; note = 2'-O-methoxyethyl 5-methylcytidine 3'-phosphorothioate
  modified_base 16: mod_base = OTHER; note = 2'-O-methoxyethyl adenosine
tcccgaatgtccgaca

SEQ ID NO: 13 - DNA, 16 bp, synthetic construct
  source 1..16: mol_type = other DNA
  modified_base 1: mod_base = OTHER; note = constrained ethyl thymidine 3'-phosphorothioate
  modified_base 2: mod_base = OTHER; note = constrained ethyl thymidine 3'-phosphodiester
  modified_base 3: mod_base = OTHER; note = constrained ethyl 5-methylcytidine 3'-phosphodiester
  modified_base 4: mod_base = OTHER; note = 5-methyl-2'-deoxycytidine 3'-phosphorothioate
  modified_base 5: mod_base = OTHER; note = 2'-O-methyl cytidine 3'-phosphorothioate
  modified_base 6: mod_base = OTHER; note = 2'-deoxyguanosine 3'-phosphorothioate
  modified_base 7..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyguanosine 3'-phosphorothioate
  modified_base 11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = constrained ethyl guanosine 3'-phosphodiester
  modified_base 15: mod_base = OTHER; note = constrained ethyl adenosine 3'-phosphorothioate
  modified_base 16: mod_base = OTHER; note = constrained ethyl 5-methylcytidine
ttcccgaatgtccgac

SEQ ID NO: 14 - DNA, 16 bp, synthetic construct
  source 1..16: mol_type = other DNA
  modified_base 5: mod_base = OTHER; note = uracil
  modified_base 1: mod_base = OTHER; note = constrained ethyl 5-methylcytidine 3'-phosphorothioate
  modified_base 2: mod_base = OTHER; note = constrained ethyl guanosine 3'-phosphodiester
  modified_base 3: mod_base = OTHER; note = constrained ethyl adenosine 3'-phosphodiester
  modified_base 4: mod_base = OTHER; note = 2'-deoxyadenosine 3'-phosphorothioate
  modified_base 5: mod_base = OTHER; note = 2'-O-methyl uridine 3'-phosphorothioate
  modified_base 6: mod_base = OTHER; note = 2'-deoxyguanosine 3'-phosphorothioate
  modified_base 7: mod_base = OTHER; note = 2'-deoxythymidine 3'-phosphorothioate
  modified_base 8..9: mod_base = OTHER; note = 5-methyl-2'-deoxycytidine 3'-phosphorothioate
  modified_base 10: mod_base = OTHER; note = 2'-deoxyguanosine 3'-phosphorothioate
  modified_base 11: mod_base = OTHER; note = 2'-deoxyadenosine 3'-phosphorothioate
  modified_base 12: mod_base = OTHER; note = 5-methyl-2'-deoxycytidine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphodiester
  modified_base 15: mod_base = OTHER; note = constrained ethyl thymidine 3'-phosphorothioate
  modified_base 16: mod_base = OTHER; note = constrained ethyl guanosine
cgaatgtccgacagtg

SEQ ID NO: 15 - DNA, 16 bp, synthetic construct
  source 1..16: mol_type = other DNA
  modified_base 1: mod_base = OTHER; note = constrained ethyl 5-methylcytidine 3'-phosphorothioate
  modified_base 2: mod_base = OTHER; note = constrained ethyl thymidine 3'-phosphodiester
  modified_base 3: mod_base = OTHER; note = 2'-O-methoxyethyl thymidine 3'-phosphodiester
  modified_base 4..5: mod_base = OTHER; note = 2'-deoxythymidine 3'-phosphorothioate
  modified_base 6: mod_base = OTHER; note = 2'-deoxyadenosine 3'-phosphorothioate
  modified_base 7..8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyguanosine 3'-phosphorothioate
  modified_base 11: mod_base = OTHER; note = 5-methyl-2'-deoxycytidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphodiester
  modified_base 15: mod_base = OTHER; note = constrained ethyl guanosine 3'-phosphorothioate
  modified_base 16: mod_base = OTHER; note = constrained ethyl guanosine
cttttattcgcgaggg

SEQ ID NO: 16 - DNA, 19 bp, synthetic construct
  source 1..19: mol_type = other DNA
gaaggtgaaggtcggagtc

SEQ ID NO: 17 - DNA, 20 bp, synthetic construct
  source 1..20: mol_type = other DNA
gaagatggtgatgggatttc

SEQ ID NO: 18 - DNA, 20 bp, synthetic construct
  source 1..20: mol_type = other DNA
caagcttcccgttctcagcc

SEQ ID NO: 19 - DNA, 16 bp, synthetic construct
  source 1..16: mol_type = other DNA
  modified_base 1: mod_base = OTHER; note = constrained ethyl adenosine 3'-phosphorothioate
  modified_base 2: mod_base = OTHER; note = constrained ethyl 5-methylcytidine 3'-phosphorothioate
  modified_base 3: mod_base = OTHER; note = constrained ethyl adenosine 3'-phosphorothioate
  modified_base 4: mod_base = OTHER; note = 2'-deoxyadenosine 3'-phosphorothioate
  modified_base 5: mod_base = OTHER; note = 2'-deoxythymidine 3'-phosphorothioate
  modified_base 6..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyadenosine 3'-phosphorothioate
  modified_base 11..12: mod_base = OTHER; note = 5-methyl-2'-deoxycytidine 3'-phosphorothioate
  modified_base 13: mod_base = OTHER; note = 2'-deoxyguanosine 3'-phosphorothioate
  modified_base 14: mod_base = OTHER; note = constrained ethyl adenosine 3'-phosphorothioate
  modified_base 15: mod_base = OTHER; note = constrained ethyl guanosine 3'-phosphorothioate
  modified_base 16: mod_base = OTHER; note = constrained ethyl guanosine
acaataaataccgagg

SEQ ID NO: 20 - DNA, 16 bp, synthetic construct
  source 1..16: mol_type = other DNA
  modified_base 1..2: mod_base = OTHER; note = constrained ethyl thymidine 3'-phosphorothioate
  modified_base 3: mod_base = OTHER; note = constrained ethyl 5-methylcytidine 3'-phosphorothioate
  modified_base 4: mod_base = OTHER; note = 5-methyl-2'-deoxycytidine 3'-phosphorothioate
  modified_base 5: mod_base = OTHER; note = 2'-O-methyl cytidine 3'-phosphorothioate
  modified_base 6: mod_base = OTHER; note = 2'-deoxyguanosine 3'-phosphorothioate
  modified_base 7..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyguanosine 3'-phosphorothioate
  modified_base 11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = constrained ethyl guanosine 3'-phosphorothioate
  modified_base 15: mod_base = OTHER; note = constrained ethyl adenosine 3'-phosphorothioate
  modified_base 16: mod_base = OTHER; note = constrained ethyl 5-methylcytidine
ttcccgaatgtccgac

SEQ ID NO: 21 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = 2'-O-methoxyethyl thymidine 3'-phosphorothioate
  modified_base 2: mod_base = OTHER; note = 2'-O-methoxyethyl 5-methylcytidine 3'-phosphodiester
  modified_base 3: mod_base = OTHER; note = constrained ethyl 5-methylcytidine 3'-phosphodiester
  modified_base 4: mod_base = OTHER; note = constrained ethyl 5-methylcytidine 3'-phosphorothioate
  modified_base 5: mod_base = OTHER; note = 2'-deoxyguanosine 3'-phosphorothioate
  modified_base 6..7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 2'-deoxyguanosine 3'-phosphorothioate
  modified_base 10: mod_base = OTHER; note = 2'-deoxythymidine 3'-phosphorothioate
  modified_base 11..12: mod_base = OTHER; note = 5-methyl-2'-deoxycytidine 3'-phosphorothioate
  modified_base 13: mod_base = OTHER; note = constrained ethyl guanosine 3'-phosphorothioate
  modified_base 14: mod_base = OTHER; note = constrained ethyl adenosine 3'-phosphodiester
  modified_base 15: mod_base = OTHER; note = 2'-O-methoxyethyl 5-methylcytidine 3'-phosphorothioate
  modified_base 16: mod_base = OTHER; note = 2'-O-methoxyethyl adenosine
tcccgaatgtccgaca

SEQ ID NO: 22 - DNA, 16 bp, synthetic construct
  source 1..16: mol_type = other DNA
  modified_base 5: mod_base = OTHER; note = Uracil
  modified_base <1: mod_base = OTHER; note = Trishexylamino-N-Acetylgalactosamine-6-aminohexyl phosphodiester
  modified_base 1: mod_base = OTHER; note = constrained ethyl 5-methylcytidine 3'-phosphorothioate
  modified_base 2: mod_base = OTHER; note = constrained ethyl guanosine 3'-phosphodiester
  modified_base 3: mod_base = OTHER; note = constrained ethyl adenosine 3'-phosphodiester
  modified_base 4: mod_base = OTHER; note = 2'-deoxyadenosine 3'-phosphorothioate
  modified_base 5: mod_base = OTHER; note = 2'-O-methyl uridine 3'-phosphorothioate
  modified_base 6: mod_base = OTHER; note = 2'-deoxyguanosine 3'-phosphorothioate
  modified_base 7: mod_base = OTHER; note = 2'-deoxythymidine 3'-phosphorothioate
  modified_base 8..9: mod_base = OTHER; note = 5-methyl-2'-deoxycytidine 3'-phosphorothioate
  modified_base 10: mod_base = OTHER; note = 2'-deoxyguanosine 3'-phosphorothioate
  modified_base 11: mod_base = OTHER; note = 2'-deoxyadenosine 3'-phosphorothioate
  modified_base 12: mod_base = OTHER; note = 5-methyl-2'-deoxycytidine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphodiester
  modified_base 15: mod_base = OTHER; note = constrained ethyl thymidine 3'-phosphorothioate
  modified_base 16: mod_base = OTHER; note = constrained ethyl guanosine
cgaatgtccgacagtg

SEQ ID NO: 23 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl thymidine 3'-phosphorothioate
  modified_base 2: mod_base = OTHER; note = constrained ethyl thymidine 3'-phosphodiester
  modified_base 3: mod_base = OTHER; note = constrained ethyl 5-methylcytidine 3'-phosphodiester
  modified_base 4: mod_base = OTHER; note = 5-methyl-2'-deoxycytidine 3'-phosphorothioate
  modified_base 5: mod_base = OTHER; note = 2'-O-methyl cytidine 3'-phosphorothioate
  modified_base 6: mod_base = OTHER; note = 2'-deoxyguanosine 3'-phosphorothioate
  modified_base 7..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyguanosine 3'-phosphorothioate
  modified_base 11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = constrained ethyl guanosine 3'-phosphodiester
  modified_base 15: mod_base = OTHER; note = constrained ethyl adenosine 3'-phosphorothioate
  modified_base 16: mod_base = OTHER; note = constrained ethyl 5-methylcytidine
ttcccgaatgtccgac

SEQ ID NO: 24 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl 5-methylcytidine 3'-phosphorothioate
  modified_base 2: mod_base = OTHER; note = constrained ethyl thymidine 3'-phosphodiester
  modified_base 3: mod_base = OTHER; note = 2'-O-methoxyethyl thymidine 3'-phosphodiester
  modified_base 4..5: mod_base = OTHER; note = 2'-deoxythymidine 3'-phosphorothioate
  modified_base 6: mod_base = OTHER; note = 2'-deoxyadenosine 3'-phosphorothioate
  modified_base 7..8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyguanosine 3'-phosphorothioate
  modified_base 11: mod_base = OTHER; note = 5-methyl-2'-deoxycytidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphodiester
  modified_base 15: mod_base = OTHER; note = constrained ethyl guanosine 3'-phosphorothioate
  modified_base 16: mod_base = OTHER; note = constrained ethyl guanosine
cttttattcgcgaggg

SEQ ID NO: 25 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = 2'-O-methoxyethyl thymidine 3'-phosphorothioate
  modified_base 2: mod_base = OTHER; note = 2'-O-methoxyethyl 5-methylcytidine 3'-phosphorothioate
  modified_base 3..4: mod_base = OTHER; note = constrained ethyl 5-methylcytidine 3'-phosphorothioate
  modified_base 5: mod_base = OTHER; note = 2'-deoxyguanosine 3'-phosphorothioate
  modified_base 6..7: mod_base = OTHER; note = 2'-deoxyadenosine 3'-phosphorothioate
  modified_base 8: mod_base = OTHER; note = 2'-deoxythymidine 3'-phosphorothioate
  modified_base 9: mod_base = OTHER; note = 2'-deoxyguanosine 3'-phosphorothioate
  modified_base 10: mod_base = OTHER; note = 2'-deoxythymidine 3'-phosphorothioate
  modified_base 11..12: mod_base = OTHER; note = 5-methyl-2'-deoxycytidine 3'-phosphorothioate
  modified_base 13: mod_base = OTHER; note = constrained ethyl guanosine 3'-phosphorothioate
  modified_base 14: mod_base = OTHER; note = constrained ethyl adenosine 3'-phosphorothioate
  modified_base 15: mod_base = OTHER; note = 2'-O-methoxyethyl 5-methylcytidine 3'-phosphorothioate
  modified_base 16: mod_base = OTHER; note = 2'-O-methoxyethyl adenosine
tcccgaatgtccgaca

SEQ ID NO: 26 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1..2: mod_base = OTHER; note = constrained ethyl thymidine 3'-phosphorothioate
  modified_base 3: mod_base = OTHER; note = constrained ethyl 5-methylcytidine 3'-phosphorothioate
  modified_base 4: mod_base = OTHER; note = 5-methyl-2'-deoxycytidine 3'-phosphorothioate
  modified_base 5: mod_base = OTHER; note = 2'-O-methyl cytidine 3'-phosphorothioate
  modified_base 6: mod_base = OTHER; note = 2'-deoxyguanosine 3'-phosphorothioate
  modified_base 7..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyguanosine 3'-phosphorothioate
  modified_base 11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = constrained ethyl guanosine 3'-phosphorothioate
  modified_base 15: mod_base = OTHER; note = constrained ethyl adenosine 3'-phosphorothioate
  modified_base 16: mod_base = OTHER; note = constrained ethyl 5-methylcytidine
ttcccgaatgtccgac

SEQ ID NO: 27 - DNA, 16 bp, synthetic construct
  source 1..16: mol_type = other DNA
  modified_base <1: mod_base = OTHER; note = Trishexylamino-N-Acetylgalactosamine-6-aminohexyl phosphodiester
  modified_base 1: mod_base = OTHER; note = constrained ethyl adenosine 3'-phosphorothioate
  modified_base 2: mod_base = OTHER; note = constrained ethyl 5-methylcytidine 3'-phosphodiester
  modified_base 3: mod_base = OTHER; note = constrained ethyl adenosine 3'-phosphodiester
  modified_base 4: mod_base = OTHER; note = 2'-deoxyadenosine 3'-phosphorothioate
  modified_base 5: mod_base = OTHER; note = 2'-deoxythymidine 3'-phosphorothioate
  modified_base 6..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyadenosine 3'-phosphorothioate
  modified_base 11..12: mod_base = OTHER; note = 5-methyl-2'-deoxycytidine 3'-phosphorothioate
  modified_base 13: mod_base = OTHER; note = 2'-deoxyguanosine 3'-phosphorothioate
  modified_base 14: mod_base = OTHER; note = constrained ethyl adenosine 3'-phosphodiester
  modified_base 15: mod_base = OTHER; note = constrained ethyl guanosine 3'-phosphorothioate
  modified_base 16: mod_base = OTHER; note = constrained ethyl guanosine
acaataaataccgagg

SEQ ID NO: 28 - DNA, 16 bp, synthetic construct
  source 1..16: mol_type = other DNA
  modified_base <1: mod_base = OTHER; note = 5'-conjugate or a Hydrogen
  modified_base 1..2: mod_base = OTHER; note = constrained ethyl thymidine 3'-phosphorothioate
  modified_base 3: mod_base = OTHER; note = constrained ethyl 5-methylcytidine 3'-phosphorothioate
  modified_base 4: mod_base = OTHER; note = 5-methyl-2'-deoxycytidine 3'-phosphorothioate
  modified_base 5: mod_base = OTHER; note = 2'-O-methyl cytidine 3'-phosphorothioate
  modified_base 6: mod_base = OTHER; note = 2'-deoxyguanosine 3'-phosphorothioate
  modified_base 7..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyguanosine 3'-phosphorothioate
  modified_base 11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = constrained ethyl guanosine 3'-phosphorothioate
  modified_base 15: mod_base = OTHER; note = constrained ethyl adenosine 3'-phosphorothioate
  modified_base 16: mod_base = OTHER; note = constrained ethyl 5-methylcytidine
  modified_base >16: mod_base = OTHER; note = 3'-conjugate or a Hydrogen
ttcccgaatgtccgac

SEQ ID NO: 29 - DNA, 16 bp, synthetic construct
  source 1..16: mol_type = other DNA
  modified_base 5: mod_base = OTHER; note = Uracil
  modified_base <1: mod_base = OTHER; note = 5'-conjugate or a Hydrogen
  modified_base 1: mod_base = OTHER; note = constrained ethyl 5-methylcytidine 3'-phosphorothioate
  modified_base 2: mod_base = OTHER; note = constrained ethyl guanosine 3'-phosphodiester
  modified_base 3: mod_base = OTHER; note = constrained ethyl adenosine 3'-phosphodiester
  modified_base 4: mod_base = OTHER; note = 2'-deoxyadenosine 3'-phosphorothioate
  modified_base 5: mod_base = OTHER; note = 2'-O-methyl uridine 3'-phosphorothioate
  modified_base 6: mod_base = OTHER; note = 2'-deoxyguanosine 3'-phosphorothioate
  modified_base 7: mod_base = OTHER; note = 2'-deoxythymidine 3'-phosphorothioate
  modified_base 8..9: mod_base = OTHER; note = 5-methyl-2'-deoxycytidine 3'-phosphorothioate
  modified_base 10: mod_base = OTHER; note = 2'-deoxyguanosine 3'-phosphorothioate
  modified_base 11: mod_base = OTHER; note = 2'-deoxyadenosine 3'-phosphorothioate
  modified_base 12: mod_base = OTHER; note = 5-methyl-2'-deoxycytidine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphodiester
  modified_base 15: mod_base = OTHER; note = constrained ethyl thymidine 3'-phosphorothioate
  modified_base 16: mod_base = OTHER; note = constrained ethyl guanosine
  modified_base >16: mod_base = OTHER; note = 3'-conjugate or a Hydrogen
cgaatgtccgacagtg

SEQ ID NO: 30 - DNA, 16 bp, synthetic construct
  source 1..16: mol_type = other DNA
  modified_base <1: mod_base = OTHER; note = 5'-conjugate or a Hydrogen
  modified_base 1: mod_base = OTHER; note = constrained ethyl thymidine 3'-phosphorothioate
  modified_base 2: mod_base = OTHER; note = constrained ethyl thymidine 3'-phosphodiester
  modified_base 3: mod_base = OTHER; note = constrained ethyl 5-methylcytidine 3'-phosphodiester
  modified_base 4: mod_base = OTHER; note = 5-methyl-2'-deoxycytidine 3'-phosphorothioate
  modified_base 5: mod_base = OTHER; note = 2'-O-methyl cytidine 3'-phosphorothioate
  modified_base 6: mod_base = OTHER; note = 2'-deoxyguanosine 3'-phosphorothioate
  modified_base 7..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyguanosine 3'-phosphorothioate
  modified_base 11: mod_base = OTHER; note = 2'-deoxythymidine 3'-phosphorothioate
  modified_base 12..13: mod_base = OTHER; note = 5-methyl-2'-deoxycytidine 3'-phosphorothioate
  modified_base 14: mod_base = OTHER; note = constrained ethyl guanosine 3'-phosphodiester
  modified_base 15: mod_base = OTHER; note = constrained ethyl adenosine 3'-phosphorothioate
  modified_base 16: mod_base = OTHER; note = constrained ethyl 5-methylcytidine
  modified_base >16: mod_base = OTHER; note = 3'-conjugate or a Hydrogen
ttcccgaatgtccgac

SEQ ID NO: 31 - DNA, 16 bp, synthetic construct
  source 1..16: mol_type = other DNA
  modified_base <1: mod_base = OTHER; note = 5'-conjugate or a Hydrogen
  modified_base 1: mod_base = OTHER; note = constrained ethyl 5-methylcytidine 3'-phosphorothioate
  modified_base 2: mod_base = OTHER; note = constrained ethyl thymidine 3'-phosphodiester
  modified_base 3: mod_base = OTHER; note = 2'-O-methoxyethyl thymidine 3'-phosphodiester
  modified_base 4..5: mod_base = OTHER; note = 2'-deoxythymidine 3'-phosphorothioate
  modified_base 6: mod_base = OTHER; note = 2'-deoxyadenosine 3'-phosphorothioate
  modified_base 7..8: mod_base = OTHER; note = 2'-deoxythymidine 3'-phosphorothioate
  modified_base 9: mod_base = OTHER; note = 5-methyl-2'-deoxycytidine 3'-phosphorothioate
  modified_base 10: mod_base = OTHER; note = 2'-deoxyguanosine 3'-phosphorothioate
  modified_base 11: mod_base = OTHER; note = 5-methyl-2'-deoxycytidine 3'-phosphorothioate
  modified_base 12: mod_base = OTHER; note = 2'-deoxyguanosine 3'-phosphorothioate
  modified_base 13: mod_base = OTHER; note = 2'-deoxyadenosine 3'-phosphorothioate
  modified_base 14: mod_base = OTHER; note = constrained ethyl guanosine 3'-phosphodiester
  modified_base 15: mod_base = OTHER; note = constrained ethyl guanosine 3'-phosphorothioate
  modified_base 16: mod_base = OTHER; note = constrained ethyl guanosine
  modified_base >16: mod_base = OTHER; note = 3'-conjugate or a Hydrogen
cttttattcgcgaggg

SEQ ID NO: 32 - DNA, 16 bp, synthetic construct
  source 1..16: mol_type = other DNA
  modified_base <1: mod_base = OTHER; note = 5'-conjugate or a Hydrogen
  modified_base 1: mod_base = OTHER; note = constrained ethyl adenosine 3'-phosphorothioate
  modified_base 2: mod_base = OTHER; note = constrained ethyl 5-methylcytidine 3'-phosphodiester
  modified_base 3: mod_base = OTHER; note = constrained ethyl adenosine 3'-phosphodiester
  modified_base 4: mod_base = OTHER; note = 2'-deoxyadenosine 3'-phosphorothioate
  modified_base 5: mod_base = OTHER; note = 2'-deoxythymidine 3'-phosphorothioate
  modified_base 6..8: mod_base = OTHER; note = 2'-deoxyadenosine 3'-phosphorothioate
  modified_base 9: mod_base = OTHER; note = 2'-deoxythymidine 3'-phosphorothioate
  modified_base 10: mod_base = OTHER; note = 2'-deoxyadenosine 3'-phosphorothioate
  modified_base 11..12: mod_base = OTHER; note = 5-methyl-2'-deoxycytidine 3'-phosphorothioate
  modified_base 13: mod_base = OTHER; note = 2'-deoxyguanosine 3'-phosphorothioate
  modified_base 14: mod_base = OTHER; note = constrained ethyl adenosine 3'-phosphodiester
  modified_base 15: mod_base = OTHER; note = constrained ethyl guanosine 3'-phosphorothioate
  modified_base 16: mod_base = OTHER; note = constrained ethyl guanosine
  modified_base >16: mod_base = OTHER; note = 3'-conjugate or a Hydrogen
acaataaataccgagg

SEQ ID NO: 33 - DNA, 16 bp, synthetic construct
  source 1..16: mol_type = other DNA
acaataaataccgagg

SEQ ID NO: 34 - DNA, 16 bp, synthetic construct
  source 1..16: mol_type = other DNA
tcccgaatgtccgaca

SEQ ID NO: 35 - DNA, 16 bp, synthetic construct
  source 1..16: mol_type = other DNA
ttcccgaatgtccgac

SEQ ID NO: 36 - DNA, 16 bp, synthetic construct
  source 1..16: mol_type = other DNA
  modified_base 5: mod_base = OTHER; note = Uracil
cgaatgtccgacagtg

SEQ ID NO: 37 - DNA, 16 bp, synthetic construct
  source 1..16: mol_type = other DNA
cttttattcgcgaggg